SEQUENCE LISTING (216 sequences)

SEQ ID NO: 1 - protein, 30 aa, Lama glama
QLQLVESGGGLVQPGGSLRVSCTASGFTFN

SEQ ID NO: 2 - protein, 5 aa, Lama glama
TYYMT

SEQ ID NO: 3 - protein, 14 aa, Lama glama
WVRQAPGKGLEWVS

SEQ ID NO: 4 - protein, 16 aa, Lama glama
DINSGGGTYYADSVKG

SEQ ID NO: 5 - protein, 32 aa, Lama glama
RFTISRDNAKNTLYLQMNSLKPEDTALYYCVR

SEQ ID NO: 6 - protein, 10 aa, Lama glama
VRIWPVGYDY

SEQ ID NO: 7 - protein, 11 aa, Lama glama
WGQGTQVTVSS

SEQ ID NO: 8 - protein, 30 aa, Lama glama
QVQLVESGGGLVQPGGSLRVSCAASGFTFS

SEQ ID NO: 9 - protein, 5 aa, Lama glama
TYYMS

SEQ ID NO: 10 - protein, 14 aa, Lama glama
WVRQAPGKGLEWVS

SEQ ID NO: 11 - protein, 16 aa, Lama glama
DIRTDGGTYYADSVKG

SEQ ID NO: 12 - protein, 32 aa, Lama glama
RFTMSRDNAKNTLYLQMNSLKPEDTALYYCAR

SEQ ID NO: 13 - protein, 10 aa, Lama glama
TRIFPSGYDY

SEQ ID NO: 14 - protein, 11 aa, Lama glama
WGQGTQVTVSS

SEQ ID NO: 15 - protein, 30 aa, Lama glama
QLQLVESGGGLVQPGGSLRLSCAASGFTFS

SEQ ID NO: 16 - protein, 5 aa, Lama glama
SHAMS

SEQ ID NO: 17 - protein, 14 aa, Lama glama
WVRQAPGKGLEWVS

SEQ ID NO: 18 - protein, 17 aa, Lama glama
AINSGGGSTSYADSVKG

SEQ ID NO: 19 - protein, 32 aa, Lama glama
RFTISRDNAKNTLYLQMNSLKPEDTAVYYCAK

SEQ ID NO: 20 - protein, 17 aa, Lama glama
ELRFDLARYTDYEAWDY

SEQ ID NO: 21 - protein, 11 aa, Lama glama
WGQGTQVTVSS

SEQ ID NO: 22 - protein, 30 aa, Lama glama
ELQLVESGGGLVQPGGSLRLSCAASGFTFS

SEQ ID NO: 23 - protein, 5 aa, Lama glama
GYGMS

SEQ ID NO: 24 - protein, 14 aa, Lama glama
WVRQAPGKGLEWVS

SEQ ID NO: 25 - protein, 17 aa, Lama glama
DINSGGGSTSYADSVKG

SEQ ID NO: 26 - protein, 32 aa, Lama glama; identical to SEQ ID NO: 19

SEQ ID NO: 27 - protein, 17 aa, Lama glama
DMRLYLARYNDYEAWDY

SEQ ID NO: 28 - protein, 11 aa, Lama glama
WGQGTQVTVSS

SEQ ID NO: 29 - protein, 30 aa, Lama glama; identical to SEQ ID NO: 22

SEQ ID NO: 30 - protein, 5 aa, Lama glama
SYGMS

SEQ ID NO: 31 - protein, 14 aa, Lama glama
WVRQAPGKGLEWVS

SEQ ID NO: 32 - protein, 17 aa, Lama glama
AINSYGGSTSYADSVKG

SEQ ID NO: 33 - protein, 32 aa, Lama glama; identical to SEQ ID NO: 19

SEQ ID NO: 34 - protein, 17 aa, Lama glama
EVRADLSRYNDYESYDY

SEQ ID NO: 35 - protein, 11 aa, Lama glama
WGQGTQVTVSS

SEQ ID NO: 36 - protein, 30 aa, Lama glama
EVQLVESGGGLVQPGGSLRLSCAASGFSFK

SEQ ID NO: 37 - protein, 5 aa, Lama glama
DYDIT

SEQ ID NO: 38 - protein, 14 aa, Lama glama
WVRQAPGKGLEWVS

SEQ ID NO: 39 - protein, 17 aa, Lama glama
TITSRSGSTSYVDSVKG

SEQ ID NO: 40 - protein, 32 aa, Lama glama
RFTISGDNAKNTLYLQMNSLKPEDTAVYYCAK

SEQ ID NO: 41 - protein, 17 aa, Lama glama
VYATTWDVGPLGYGMDY

SEQ ID NO: 42 - protein, 11 aa, Lama glama
WGKGTLVTVSS

SEQ ID NO: 43 - protein, 30 aa, Lama glama
EVQLQESGGGLVQPGGSLRLSCAASGFTFS

SEQ ID NO: 44 - protein, 5 aa, Lama glama
IYDMS

SEQ ID NO: 45 - protein, 14 aa, Lama glama
WVRQAPGKGLEWVS

SEQ ID NO: 46 - protein, 17 aa, Lama glama
TINSDGSSTSYVDSVKG

SEQ ID NO: 47 - protein, 32 aa, Lama glama; identical to SEQ ID NO: 19

SEQ ID NO: 48 - protein, 17 aa, Lama glama
VYGSTWDVGPMGYGMDY

SEQ ID NO: 49 - protein, 11 aa, Lama glama
WGKGTLVTVSS

SEQ ID NO: 50 - protein, 30 aa, Lama glama
QVQLVESGGNLVQPGGSLRLSCAASGFTFS

SEQ ID NO: 51 - protein, 5 aa, Lama glama
NYYMS

SEQ ID NO: 52 - protein, 14 aa, Lama glama
WVRQAPGKGLEWVS

SEQ ID NO: 53 - protein, 17 aa, Lama glama
DIYSDGSTTWYSDSVKG

SEQ ID NO: 54 - protein, 32 aa, Lama glama
RFTISRDNAKNTLSLQMNSLKSEDTAVYYCAR

SEQ ID NO: 55 - protein, 10 aa, Lama glama
VKIYPGGYDA

SEQ ID NO: 56 - protein, 11 aa, Lama glama
WGQGTQVTVSS

SEQ ID NO: 57 - protein, 30 aa, Lama glama
QVQLQESGGDLVQPGGSLRVSCVVSGFTFS

SEQ ID NO: 58 - protein, 5 aa, Lama glama
RYYMS

SEQ ID NO: 59 - protein, 14 aa, Lama glama
WVRQAPGKGLEWVS

SEQ ID NO: 60 - protein, 17 aa, Lama glama
SIDSYGYSTYYTDSVKG

SEQ ID NO: 61 - protein, 32 aa, Lama glama
RFTISRDNAKNTLYLQMNSLKPEDTALYYCAR

SEQ ID NO: 62 - protein, 9 aa, Lama glama
AKTTWSYDY

SEQ ID NO: 63 - protein, 11 aa, Lama glama
WGQGTQVTVSS

SEQ ID NO: 64 - protein, 30 aa, Lama glama
EVQLVESGGGLVQPGGSLRLSCAASGFTFR

SEQ ID NO: 65 - protein, 5 aa, Lama glama
NYHMS

SEQ ID NO: 66 - protein, 14 aa, Lama glama
WVRQVPGKGFEWIS

SEQ ID NO: 67 - protein, 17 aa, Lama glama
DINSAGGSTYYADSVKG

SEQ ID NO: 68 - protein, 32 aa, Lama glama
RFTISRDNAKNTLYLEMNSLKPEDTALYYCAR

SEQ ID NO: 69 - protein, 8 aa, Lama glama
VNVWGVNY

SEQ ID NO: 70 - protein, 11 aa, Lama glama
WGKGTLVSVSS

SEQ ID NO: 71 - protein, 30 aa, Lama glama; identical to SEQ ID NO: 22

SEQ ID NO: 72 - protein, 5 aa, Lama glama
NYVMS

SEQ ID NO: 73 - protein, 14 aa, Lama glama
WVRQAPGKGLEWVS

SEQ ID NO: 74 - protein, 16 aa, Lama glama
DTNSGGSTSYADSVKG

SEQ ID NO: 75 - protein, 32 aa, Lama glama; identical to SEQ ID NO: 61

SEQ ID NO: 76 - protein, 8 aa, Lama glama
SFFYGMNY

SEQ ID NO: 77 - protein, 11 aa, Lama glama
WGKGTQVTVSS

SEQ ID NO: 78 - protein, 22 aa, Lama glama
QAVVTQEPSLSVSPGGTVTLTC

SEQ ID NO: 79 - protein, 14 aa, Lama glama
GLSSGSVTTSNYPG

SEQ ID NO: 80 - protein, 15 aa, Lama glama
WFQQTPGQAPRTLIY

SEQ ID NO: 81 - protein, 7 aa, Lama glama
NTNNRHS

SEQ ID NO: 82 - protein, 32 aa, Lama glama
GVPSRFSGSISGNKAALTITGAQPEDEADYYC

SEQ ID NO: 83 - protein, 10 aa, Lama glama
SLYTGSYTTV

SEQ ID NO: 84 - protein, 10 aa, Lama glama
FGGGTHLTVL

SEQ ID NO: 85 - protein, 22 aa, Lama glama
QAVVTQEPSLSVSPGGTVTLTC

SEQ ID NO: 86 - protein, 14 aa, Lama glama
GLSSGSVTTSNYPG

SEQ ID NO: 87 - protein, 15 aa, Lama glama
WFQQTPGQAPRTLIY

SEQ ID NO: 88 - protein, 7 aa, Lama glama
NTNSRHS

SEQ ID NO: 89 - protein, 32 aa, Lama glama
GVPSRFSGSISGNKAALTIMGAQPEDEADYYC

SEQ ID NO: 90 - protein, 9 aa, Lama glama
SLYPGSTTV

SEQ ID NO: 91 - protein, 10 aa, Lama glama
FGGGTHLTVL

SEQ ID NO: 92 - protein, 22 aa, Lama glama
SYELTQPSALSVTLGQTAKITC

SEQ ID NO: 93 - protein, 11 aa, Lama glama
QGGSLGSSYAH

SEQ ID NO: 94 - protein, 15 aa, Lama glama
WYQQKPGQAPVLVIY

SEQ ID NO: 95 - protein, 7 aa, Lama glama
DDDSRPS

SEQ ID NO: 96 - protein, 32 aa, Lama glama
GIPERFSGSSSGGTATLTISGAQAEDEGDYYC

SEQ ID NO: 97 - protein, 11 aa, Lama glama
QSADSSGNAAV

SEQ ID NO: 98 - protein, 10 aa, Lama glama
FGGGTHLTVL

SEQ ID NO: 99 - protein, 22 aa, Lama glama
SSALTQPSALSVTLGQTAKITC

SEQ ID NO: 100 - protein, 11 aa, Lama glama
QGGSLGSSYAH

SEQ ID NO: 101 - protein, 15 aa, Lama glama
WYQQKPGQAPVLVIY

SEQ ID NO: 102 - protein, 7 aa, Lama glama
DDDSRPS

SEQ ID NO: 103 - protein, 32 aa, Lama glama; identical to SEQ ID NO: 96

SEQ ID NO: 104 - protein, 11 aa, Lama glama
QSADSSGNAAV

SEQ ID NO: 105 - protein, 10 aa, Lama glama
FGGGTHLTVL

SEQ ID NO: 106 - protein, 22 aa, Lama glama
QPVLNQPSALSVTLGQTAKITC

SEQ ID NO: 107 - protein, 11 aa, Lama glama
QGGSLGARYAH

SEQ ID NO: 108 - protein, 15 aa, Lama glama
WYQQKPGQAPVLVIY

SEQ ID NO: 109 - protein, 7 aa, Lama glama
DDDSRPS

SEQ ID NO: 110 - protein, 32 aa, Lama glama; identical to SEQ ID NO: 96

SEQ ID NO: 111 - protein, 9 aa, Lama glama
QSADSSGSV

SEQ ID NO: 112 - protein, 10 aa, Lama glama
FGGGTHLTVL

SEQ ID NO: 113 - protein, 22 aa, Lama glama
SYELTQPSALSVTLGQTAKITC

SEQ ID NO: 114 - protein, 11 aa, Lama glama
QGGSLGSSYAH

SEQ ID NO: 115 - protein, 15 aa, Lama glama
WYQQKPGQAPVLVIY

SEQ ID NO: 116 - protein, 7 aa, Lama glama
DDDSRPS

SEQ ID NO: 117 - protein, 32 aa, Lama glama; identical to SEQ ID NO: 96

SEQ ID NO: 118 - protein, 11 aa, Lama glama
QSADSSGNAAV

SEQ ID NO: 119 - protein, 10 aa, Lama glama
FGGGTHLTVL

SEQ ID NO: 120 - protein, 22 aa, Lama glama
SSALTQPSALSVSLGQTARITC

SEQ ID NO: 121 - protein, 11 aa, Lama glama
QGGSLGSSYAH

SEQ ID NO: 122 - protein, 15 aa, Lama glama
WYQQKPGQAPVLVIY

SEQ ID NO: 123 - protein, 7 aa, Lama glama
GDDSRPS

SEQ ID NO: 124 - protein, 32 aa, Lama glama
GIPERFSGSSSGGTATLTISGAQAEDEDDYYC

SEQ ID NO: 125 - protein, 10 aa, Lama glama
QSTDSSGNTV

SEQ ID NO: 126 - protein, 10 aa, Lama glama
FGGGTRLTVL

SEQ ID NO: 127 - protein, 22 aa, Lama glama
QAGLTQPPSVSGSPGKTVTISC

SEQ ID NO: 128 - protein, 14 aa, Lama glama
AGNSSDVGYGNYVS

SEQ ID NO: 129 - protein, 15 aa, Lama glama
WYQQFPGMAPKLLIY

SEQ ID NO: 130 - protein, 7 aa, Lama glama
LVNKRAS

SEQ ID NO: 131 - protein, 32 aa, Lama glama
GITDRFSGSKSGNTASLTISGLQSEDEADYYC

SEQ ID NO: 132 - protein, 10 aa, Lama glama
ASYTGSNNIV

SEQ ID NO: 133 - protein, 10 aa, Lama glama
FGGGTHLTVL

SEQ ID NO: 134 - protein, 23 aa, Lama glama
EIVLTQSPSSVTASVGGKVTINC

SEQ ID NO: 135 - protein, 17 aa, Lama glama
KSSQSVFIASNQKTYLN

SEQ ID NO: 136 - protein, 15 aa, Lama glama
WYQQRPGQSPRLVIS

SEQ ID NO: 137 - protein, 7 aa, Lama glama
YASTRES

SEQ ID NO: 138 - protein, 32 aa, Lama glama
GIPDRFSGSGSTTDFTLTISSVQPEDAAVYYC

SEQ ID NO: 139 - protein, 8 aa, Lama glama
QQAYSHPT

SEQ ID NO: 140 - protein, 10 aa, Lama glama
FGQGTKVELK

SEQ ID NO: 141 - protein, 22 aa, Lama glama
QTVVTQEPSLSVSPGGTVTLTC

SEQ ID NO: 142 - protein, 14 aa, Lama glama
GLSSGSVTTSNYPG

SEQ ID NO: 143 - protein, 15 aa, Lama glama
WFQQTPGQAPRTLIY

SEQ ID NO: 144 - protein, 7 aa, Lama glama
NTNSRHS

SEQ ID NO: 145 - protein, 32 aa, Lama glama; identical to SEQ ID NO: 82

SEQ ID NO: 146 - protein, 10 aa, Lama glama
SLYPGSYTNV

SEQ ID NO: 147 - protein, 10 aa, Lama glama
FGGGTHLTVL

SEQ ID NO: 148 - protein, 22 aa, Lama glama
QSALTQPPSLSASPGSSVRLTC

SEQ ID NO: 149 - protein, 14 aa, Lama glama
TLSSGNNIGSYDIS

SEQ ID NO: 150 - protein, 15 aa, Lama glama
WYQQKAGSPPRYLLN

SEQ ID NO: 151 - protein, 11 aa, Lama glama
YYTDSRKHQDS

SEQ ID NO: 152 - protein, 34 aa, Lama glama
GVPSRFSGSKDASANAGLLLISGLQPEDEADYYC

SEQ ID NO: 153 - protein, 11 aa, Lama glama
SAYKSGSYRWV

SEQ ID NO: 154 - protein, 10 aa, Lama glama
FGGGTHVTVL

SEQ ID NO: 155 - protein, 118 aa, Lama glama
QLQLVESGGGLVQPGGSLRVSCTASGFTFNTYYMTWVRQAPGKGLEWVSDINSGGGTYYADSVKGRFTISRDNAKNTLYLQMNSLKPEDTALYYCVRVRIWPVGYDYWGQGTQVTVSS

SEQ ID NO: 156 - protein, 110 aa, Lama glama
QAVVTQEPSLSVSPGGTVTLTCGLSSGSVTTSNYPGWFQQTPGQAPRTLIYNTNNRHSGVPSRFSGSISGNKAALTITGAQPEDEADYYCSLYTGSYTTVFGGGTHLTVL

SEQ ID NO: 157 - protein, 118 aa, Lama glama
QVQLVESGGGLVQPGGSLRVSCAASGFTFSTYYMSWVRQAPGKGLEWVSDIRTDGGTYYADSVKGRFTMSRDNAKNTLYLQMNSLKPEDTALYYCARTRIFPSGYDYWGQGTQVTVSS

SEQ ID NO: 158 - protein, 109 aa, Lama glama
QAVVTQEPSLSVSPGGTVTLTCGLSSGSVTTSNYPGWFQQTPGQAPRTLIYNTNSRHSGVPSRFSGSISGNKAALTIMGAQPEDEADYYCSLYPGSTTVFGGGTHLTVL

SEQ ID NO: 159 - protein, 126 aa, Lama glama
QLQLVESGGGLVQPGGSLRLSCAASGFTFSSHAMSWVRQAPGKGLEWVSAINSGGGSTSYADSVKGRFTISRDNAKNTLYLQMNSLKPEDTAVYYCAKELRFDLARYTDYEAWDYWGQGTQVTVSS

SEQ ID NO: 160 - protein, 108 aa, Lama glama
SYELTQPSALSVTLGQTAKITCQGGSLGSSYAHWYQQKPGQAPVLVIYDDDSRPSGIPERFSGSSSGGTATLTISGAQAEDEGDYYCQSADSSGNAAVFGGGTHLTVL

SEQ ID NO: 161 - protein, 126 aa, Lama glama
ELQLVESGGGLVQPGGSLRLSCAASGFTFSGYGMSWVRQAPGKGLEWVSDINSGGGSTSYADSVKGRFTISRDNAKNTLYLQMNSLKPEDTAVYYCAKDMRLYLARYNDYEAWDYWGQGTQVTVSS

SEQ ID NO: 162 - protein, 108 aa, Lama glama
SSALTQPSALSVTLGQTAKITCQGGSLGSSYAHWYQQKPGQAPVLVIYDDDSRPSGIPERFSGSSSGGTATLTISGAQAEDEGDYYCQSADSSGNAAVFGGGTHLTVL

SEQ ID NO: 163 - protein, 126 aa, Lama glama
ELQLVESGGGLVQPGGSLRLSCAASGFTFSSYGMSWVRQAPGKGLEWVSAINSYGGSTSYADSVKGRFTISRDNAKNTLYLQMNSLKPEDTAVYYCAKEVRADLSRYNDYESYDYWGQGTQVTVSS

SEQ ID NO: 164 - protein, 106 aa, Lama glama
QPVLNQPSALSVTLGQTAKITCQGGSLGARYAHWYQQKPGQAPVLVIYDDDSRPSGIPERFSGSSSGGTATLTISGAQAEDEGDYYCQSADSSGSVFGGGTHLTVL

SEQ ID NO: 165 - protein, 126 aa, Lama glama
EVQLVESGGGLVQPGGSLRLSCAASGFSFKDYDITWVRQAPGKGLEWVSTITSRSGSTSYVDSVKGRFTISGDNAKNTLYLQMNSLKPEDTAVYYCAKVYATTWDVGPLGYGMDYWGKGTLVTVSS

SEQ ID NO: 166 - protein, 108 aa, Lama glama; identical to SEQ ID NO: 160

SEQ ID NO: 167 - protein, 126 aa, Lama glama
EVQLQESGGGLVQPGGSLRLSCAASGFTFSIYDMSWVRQAPGKGLEWVSTINSDGSSTSYVDSVKGRFTISRDNAKNTLYLQMNSLKPEDTAVYYCAKVYGSTWDVGPMGYGMDYWGKGTLVTVSS

SEQ ID NO: 168 - protein, 107 aa, Lama glama
SSALTQPSALSVSLGQTARITCQGGSLGSSYAHWYQQKPGQAPVLVIYGDDSRPSGIPERFSGSSSGGTATLTISGAQAEDEDDYYCQSTDSSGNTVFGGGTRLTVL

SEQ ID NO: 169 - protein, 119 aa, Lama glama
QVQLVESGGNLVQPGGSLRLSCAASGFTFSNYYMSWVRQAPGKGLEWVSDIYSDGSTTWYSDSVKGRFTISRDNAKNTLSLQMNSLKSEDTAVYYCARVKIYPGGYDAWGQGTQVTVSS

SEQ ID NO: 170 - protein, 110 aa, Lama glama
QAGLTQPPSVSGSPGKTVTISCAGNSSDVGYGNYVSWYQQFPGMAPKLLIYLVNKRASGITDRFSGSKSGNTASLTISGLQSEDEADYYCASYTGSNNIVFGGGTHLTVL

SEQ ID NO: 171 - protein, 118 aa, Lama glama
QVQLQESGGDLVQPGGSLRVSCVVSGFTFSRYYMSWVRQAPGKGLEWVSSIDSYGYSTYYTDSVKGRFTISRDNAKNTLYLQMNSLKPEDTALYYCARAKTTWSYDYWGQGTQVTVSS

SEQ ID NO: 172 - protein, 112 aa, Lama glama
EIVLTQSPSSVTASVGGKVTINCKSSQSVFIASNQKTYLNWYQQRPGQSPRLVISYASTRESGIPDRFSGSGSTTDFTLTISSVQPEDAAVYYCQQAYSHPTFGQGTKVELK

SEQ ID NO: 173 - protein, 117 aa, Lama glama
EVQLVESGGGLVQPGGSLRLSCAASGFTFRNYHMSWVRQVPGKGFEWISDINSAGGSTYYADSVKGRFTISRDNAKNTLYLEMNSLKPEDTALYYCARVNVWGVNYWGKGTLVSVSS

SEQ ID NO: 174 - protein, 110 aa, Lama glama
QTVVTQEPSLSVSPGGTVTLTCGLSSGSVTTSNYPGWFQQTPGQAPRTLIYNTNSRHSGVPSRFSGSISGNKAALTITGAQPEDEADYYCSLYPGSYTNVFGGGTHLTVL

SEQ ID NO: 175 - protein, 116 aa, Lama glama
ELQLVESGGGLVQPGGSLRLSCAASGFTFSNYVMSWVRQAPGKGLEWVSDTNSGGSTSYADSVKGRFTISRDNAKNTLYLQMNSLKPEDTALYYCARSFFYGMNYWGKGTQVTVSS

SEQ ID NO: 176 - protein, 117 aa, Lama glama
QSALTQPPSLSASPGSSVRLTCTLSSGNNIGSYDISWYQQKAGSPPRYLLNYYTDSRKHQDSGVPSRFSGSKDASANAGLLLISGLQPEDEADYYCSAYKSGSYRWVFGGGTHVTVL

SEQ ID NO: 177 - DNA, 354 bp, Lama glama
cagttgcagctggtggagtctgggggaggcttggtgcagcctggggggtctctgagagtttcctgtacagcctctggattcaccttcaatacctactacatgacctgggtccgccaggctccagggaaggggctcgagtgggtctcagatattaatagtggtggtggtacatactatgcagactccgtgaagggccgattcaccatctccagagacaacgccaagaacacgctatatctgcaaatgaacagcctgaaacctgaggacacggccctgtattactgtgtaagagttcgtatttggccagtgggatatgactactggggccaggggacccaggtcaccgtttcctca

SEQ ID NO: 178 - DNA, 330 bp, Lama glama
caggctgtggtgacccaggagccgtccctgtcagtgtctccaggagggacggtcacactcacctgcggcctcagctctgggtctgtcactaccagtaactaccctggttggttccagcagacaccgggccaggctccacgcactcttatctacaacacaaacaaccgccactctggggtccccagtcgcttctccggatccatctctgggaacaaagccgccctcaccatcacgggggcccagcccgaggacgaggccgactattactgttctctatatactggcagttacactactgtgttcggcggagggacccatctgaccgtcctg

SEQ ID NO: 179 - DNA, 354 bp, Lama glama
caggtgcagctcgtggagtctgggggaggcttggtgcagcctggggggtctctgagagtctcctgtgcagcctctggattcaccttcagtacctactacatgagctgggtccgccaggctccagggaaggggctcgagtgggtctcagatattcgtactgatggtggcacatactatgcagactccgtgaagggccgattcaccatgtccagagacaacgccaagaacacgctgtatctacaaatgaacagcctgaaacctgaggacacggccctgtattactgtgcaagaactcgaattttcccctcggggtatgactactggggccaggggacccaggtcaccgtctcctca

SEQ ID NO: 180 - DNA, 327 bp, Lama glama
caggctgtggtgacccaggagccgtccctgtcagtgtctccaggagggacggtcacactcacctgcggcctcagctctgggtctgtcactaccagtaactaccctggttggttccagcagacaccaggccaggctccgcgcactcttatctacaacacaaacagccgccactctggggtccccagtcgcttctccggatccatctctgggaacaaagccgccctcaccatcatgggggcccagcccgaggacgaggccgactattactgttctctgtaccctggtagtaccactgtgttcggcggagggacccatctgaccgtcctg

SEQ ID NO: 181 - DNA, 378 bp, Lama glama
cagttgcagctggtggagtctgggggaggcttggtgcagcctggggggtctctgagactctcctgtgcagcctctggattcaccttcagtagccatgccatgagctgggtccgccaggctccaggaaaggggctcgagtgggtctcagctattaatagtggtggtggtagcacaagctatgcagactccgtgaagggccgattcaccatctccagagacaacgccaagaacacgctgtacctgcaaatgaacagcctgaaacctgaggacacggccgtgtattactgtgcaaaagagctgagattcgacctagcaaggtataccgactatgaggcctgggactactggggccaggggacccaggtcaccgtctcctca

SEQ ID NO: 182 - DNA, 324 bp, Lama glama
tcctatgagctgactcagccctccgcgctgtccgtaaccttgggacagacggccaagatcacctgccaaggtggcagcttaggtagcagttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggtaatgctgctgtgttcggcggagggacccatctgaccgtcctg

SEQ ID NO: 183 - DNA, 378 bp, Lama glama
gagttgcagctggtggagtctgggggaggcttggtgcagcctggggggtctctgagactctcctgtgcagcctctggattcaccttcagtggctatggcatgagctgggtccgccaggctccaggaaaggggctcgagtgggtctcagatattaatagtggtggtggtagcacaagctatgcagactccgtgaagggccgattcaccatctccagagacaacgccaagaacacgctgtatctgcaaatgaacagcctgaaacctgaggacacggccgtgtattactgtgcaaaagatatgagattatacctagcaaggtataacgactatgaggcctgggactactggggccaggggacccaggtcaccgtctcctca

SEQ ID NO: 184 - DNA, 324 bp, Lama glama
tcctctgcactgactcagccctccgcgctgtccgtaaccttgggacagacggccaagatcacctgccaaggtggcagcttaggtagcagttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggtaatgctgctgtgttcggcggagggacccatctgaccgtcctg

SEQ ID NO: 185 - DNA, 378 bp, Lama glama
gagttgcagctggtggagtctgggggaggcttggtgcagcctggggggtctctgagactctcctgtgcagcctctggattcaccttcagtagctatggcatgagctgggtccgccaggctccaggaaaggggctcgagtgggtctcagctattaatagttatggtggtagcacaagctatgcagactccgtgaagggccgattcaccatctccagagacaacgccaagaacacgctgtatctgcaaatgaacagcctgaaacctgaggacacggccgtgtattactgtgcaaaagaagtgcgggccgacctaagccgctataacgactatgagtcgtatgactactggggccaggggacccaggtcaccgtctcctca

SEQ ID NO: 186 - DNA, 318 bp, Lama glama
cagccggtgctgaatcagccctccgcgctgtccgtaaccttgggacagacggccaagatcacctgccaaggtggcagcttaggtgcgcgttatgctcactggtaccagcagaagccaggccaggcccctgtgctggtcatctatgatgatgacagcaggccctcagggatccctgagcggttctctggctccagctctgggggcacagccaccctgaccatcagcggggcccaggccgaggacgagggtgactattactgtcagtcagcagacagcagtggttctgtgttcggcggagggacccatctgaccgtcctg

SEQ ID NO: 187 - DNA, 378 bp, Lama glama
gaggtgcagctcgtggagtctgggggaggcttggtgcagcctggggggtctctgagactctcctgtgcagcctctggattcagcttcaaggactatgacataacctgggtccgccaggctccgggaaaggggctcgagtgggtctcaactattactagtcgtagtggtagcacaagctatgtagactccgtaaagggccgattcaccatctccggagacaacgccaagaacacgctgtatctgcaaatgaacagcctgaaacctgaggacacggccgtgtattactgtgcaaaagtttacgcgactacctgggacgtcggccctctgggctacggcatggactactggggcaaggggaccctggtcaccgtctcctca

SEQ ID NO: 188 - DNA, 324 bp, Lama glama; identical to SEQ ID NO: 182

SEQ ID NO: 189 - DNA, 378 bp, Lama glama
gaggtgcagctgcaggagtcggggggaggcttggtgcagcctggggggtctctgagactctcctgtgcagcctctggattcaccttcagtatatatgacatgagctgggtccgccaggctccaggaaaggggctcgagtgggtctcaactattaatagtgatggtagtagcacaagctatgtagactccgtgaagggccgattcaccatctccagagacaacgccaagaacacgctgtatctgcaaatgaacagcctgaaacctgaggacacggccgtgtattactgtgcgaaagtttacggtagtacctgggacgtcggccctatgggctacggcatggactactggggcaaagggaccctggtcactgtctcctca

SEQ ID NO: 190 - DNA, 321 bp, Lama glama
tcctctgcactgactcagccctccgcgctgtccgtgtccttgggacagacggccaggatcacctgccaaggtggcagcttaggtagcagttatgctcactggtaccagcagaagccaggccaggcccctgtgctggtcatctatggtgatgacagcaggccctcagggatccctgagcggttctctggctccagctctgggggcacagccaccctgaccatcagcggggcccaggccgaggacgaggatgactattactgtcagtcaacagacagcagtggtaatactgtgttcggcggagggacccgactgaccgtcctg

SEQ ID NO: 191 - DNA, 357 bp, Lama glama
caggtgcagctggtggagtctgggggaaacttggtgcagcctgggggttctctgagactctcctgtgcagcctctggattcaccttcagtaactactacatgagctgggtccgccaggctccagggaaggggctggaatgggtgtccgatatttatagtgacggtagtaccacatggtattcagactccgtcaagggccgattcaccatctccagagacaacgccaagaacacgctgtctctgcaaatgaacagtctgaaatctgaggacacggccgtctattactgtgcgcgcgtgaagatctatccgggggggtatgacgcctggggccaggggacccaggtcaccgtctcctca

SEQ ID NO: 192 - DNA, 330 bp, Lama glama
caggcagggctgactcagcctccctccgtgtctgggtctccaggaaagacggtcaccatctcctgtgcaggaaacagcagtgatgttgggtatggaaactatgtctcctggtaccagcagttcccaggaatggcccccaaactcctgatatatctcgtcaataaacgggcctcagggatcactgatcgcttctctggctccaagtcaggcaacacggcctccctgaccatctctgggctccagtctgaggacgaggctgattattactgtgcctcatatacaggtagcaacaatatcgtgttcggcggagggacccatctaaccgtcctc

SEQ ID NO: 193 - DNA, 354 bp, Lama glama
caggtgcagctgcaggagtcggggggagacttggtgcagcctggggggtctctgagagtctcctgtgtagtctctggattcaccttcagtcgctactacatgagctgggtccgccaggctccagggaaggggctcgagtgggtctcatctattgatagttatggttacagcacatactatacagactccgtgaagggccgattcaccatctccagagacaacgccaagaacacgctgtatctgcaaatgaacagcctgaaacctgaggacacggccctgtattactgtgcaagagcgaaaacgacttggagttatgactactggggccaggggacccaggtcaccgtctcctca

SEQ ID NO: 194 - DNA, 336 bp, Lama glama
gaaattgtgttgacgcagtctcccagctccgtgactgcatctgtaggagggaaggtcactatcaactgtaagtccagccagagcgtcttcatagcttctaatcagaaaacctacttaaactggtaccagcagagacctggacagtctccgaggttggtcatcagctatgcgtccacccgtgaatcggggatccctgatcgattcagcggcagtgggtccacaacagatttcactctcacgatcagcagtgtccagcctgaagatgcggccgtgtattactgtcagcaggcttatagccatccaacgttcggccaggggaccaaggtggaactcaaa

SEQ ID NO: 195 - DNA, 351 bp, Lama glama
gaggtgcagctcgtggagtctgggggaggcttggtgcaacctgggggttctctgagactctcctgtgcagcctctggattcaccttcaggaattaccacatgagttgggtccgccaggttccagggaaggggttcgagtggatctcagatattaatagtgcaggtggtagcacatactatgcagactccgtgaagggccgattcaccatctccagagacaacgccaagaacacgctgtatctggaaatgaacagcctgaaacctgaggacacggccctgtattactgtgcaagagtcaacgtctggggggtgaactactggggcaaagggaccctggtcagcgtctcctca

SEQ ID NO: 196 - DNA, 330 bp, Lama glama
cagactgtggtgactcaggagccgtccctgtcagtgtctccaggagggacggtcacactcacctgcggcctcagctctgggtctgtcactaccagtaactaccctggttggttccagcagacaccaggccaggctccacgcactcttatctacaacacaaacagccgccactctggggtccccagtcgcttctccggatccatctctgggaacaaagccgccctcaccatcacgggggcccagcccgaggacgaggccgactattactgttctctgtaccctggtagttacactaatgtgttcggcggagggacccatctgaccgtcctg

SEQ ID NO: 197 - DNA, 348 bp, Lama glama
gagttgcagctggtggagtctgggggaggcttggtgcagcctggggggtctctgagactctcctgtgcagcctctggattcaccttcagcaactatgtcatgagctgggtccgccaggctccaggaaaggggctcgagtgggtctcagatactaatagtggtggtagcacaagctatgcagactccgtgaagggccgattcaccatctctagagacaacgccaagaacacgctgtatttgcaaatgaacagcctgaaacctgaggacacggcattgtattactgtgcgagatcatttttctacggcatgaactactggggcaaagggacccaggtcaccgtgtcctca

SEQ ID NO: 198 - DNA, 351 bp, Lama glama
cagtctgccctgactcagccgccctccctctctgcatctccgggatcatctgtcagactcacctgcaccctgagcagtggaaacaatattggcagctatgacataagttggtaccagcagaaggcagggagccctccccggtacctcctgaactactacaccgactcacgcaagcaccaggactccggggtcccgagccgcttctctgggtccaaagatgcctcggccaacgcagggcttctgctcatctctgggcttcagcccgaggacgaggctgactattactgttctgcatacaagagtggttcttaccgttgggtgttcggcggagggacgcacgtgaccgtcctg

SEQ ID NO: 199 - protein, 10 aa, Homo sapiens
EPKSCDKTHT

SEQ ID NO: 200 - protein, 5 aa, Homo sapiens
CPPCP

SEQ ID NO: 201 - protein, 8 aa, Homo sapiens
APELLGGP

SEQ ID NO: 202 - protein, 12 aa, Homo sapiens
ELKTPLGDTTHT

SEQ ID NO: 203 - protein, 5 aa, Homo sapiens
CPRCP

SEQ ID NO: 204 - protein, 8 aa, Homo sapiens
APELLGGP

SEQ ID NO: 205 - protein, 7 aa, Homo sapiens
ESKYGPP

SEQ ID NO: 206 - protein, 5 aa, Homo sapiens
CPSCP

SEQ ID NO: 207 - protein, 8 aa, Homo sapiens
APEFLGGP

SEQ ID NO: 208 - protein, 3 aa, Homo sapiens
ERK

SEQ ID NO: 209 - protein, 10 aa, Homo sapiens
CCVECPPPCP

SEQ ID NO: 210 - protein, 7 aa, Homo sapiens
APPVAGP

SEQ ID NO: 211 - protein, 442 aa, Artificial Sequence
  Full heavy chain of llama-mouse chimeric 71G3
QVQLVESGGGLVQPGGSLRVSCAASGFTFSTYYMSWVRQAPGKGLEWVSDIRTDGGTYYADSVKGRFTMSRDNAKNTLYLQMNSLKPEDTALYYCARTRIFPSGYDYWGQGTQVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 212 - protein, 219 aa, Artificial Sequence
  Full  light chain of llama-mouse chimeric 71G3
QAVVTQEPSLSVSPGGTVTLTCGLSSGSVTTSNYPGWFQQTPGQAPRTLIYNTNSRHSGVPSRFSGSISGNKAALTIMGAQPEDEADYYCSLYPGSTTVFGGGTHLTVLGQPKSSPSVTLFPPSSEELETNKATLVCTITDFYPGVVTVDWKVDGTPVTQGMETETTQPSKQSNNKYMETASSYLTLTARAWERHSSYSCQVTHEGHTVEKSLSRADCS

SEQ ID NO: 213 - protein, 450 aa, Artificial Sequence
  Full heavy chain of llama-mouse chimeric 71D6
ELQLVESGGGLVQPGGSLRLSCAASGFTFSSYGMSWVRQAPGKGLEWVSAINSYGGSTSYADSVKGRFTISRDNAKNTLYLQMNSLKPEDTAVYYCAKEVRADLSRYNDYESYDYWGQGTQVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 214 - protein, 216 aa, Artificial Sequence
  Full light chain of llama-mouse chimeric 71D6
QPVLNQPSALSVTLGQTAKITCQGGSLGARYAHWYQQKPGQAPVLVIYDDDSRPSGIPERFSGSSSGGTATLTISGAQAEDEGDYYCQSADSSGSVFGGGTHLTVLGQPKSSPSVTLFPPSSEELETNKATLVCTITDFYPGVVTVDWKVDGTPVTQGMETETTQPSKQSNNKYMETASSYLTLTARAWERHSSYSCQVTHEGHTVEKSLSRADCS

SEQ ID NO: 215 - protein, 450 aa, Artificial Sequence
  Full heavy chain of llama-mouse chimeric 71G2
EVQLQESGGGLVQPGGSLRLSCAASGFTFSIYDMSWVRQAPGKGLEWVSTINSDGSSTSYVDSVKGRFTISRDNAKNTLYLQMNSLKPEDTAVYYCAKVYGSTWDVGPMGYGMDYWGKGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 216 - protein, 217 aa, Artificial Sequence
  Full light chain of llama-mouse chimeric 71G2
SSALTQPSALSVSLGQTARITCQGGSLGSSYAHWYQQKPGQAPVLVIYGDDSRPSGIPERFSGSSSGGTATLTISGAQAEDEDDYYCQSTDSSGNTVFGGGTRLTVLGQPKSSPSVTLFPPSSEELETNKATLVCTITDFYPGVVTVDWKVDGTPVTQGMETETTQPSKQSNNKYMETASSYLTLTARAWERHSSYSCQVTHEGHTVEKSLSRADCS

(19) United States
(12) Patent Application Publication
(10) Pub. No.: US 2020/0407452 A1
(43) Pub. Date: Dec. 31, 2020

(54) METHODS FOR PROMOTING PANCREATIC ISLET CELL GROWTH

(71) Applicant: AGOMAB THERAPEUTICS, Gent (BE)

(72) Inventor: Paolo MICHIELI, Rivalta di Torino (IT)

(21) Appl. No.: 16/921,695

(22) Filed: Jul. 6, 2020

Related U.S. Application Data
(63) Continuation of application No. PCT/EP2019/050084, filed on Jan. 3, 2019.
Application No. PCT/EP2019/050084 is a continuation-in-part of application No. 16/313,710, filed on Dec. 27, 2018.

(30) Foreign Application Priority Data
Jun. 27, 2016 (GB) 1611123.9
Jan. 3, 2018 (IT) 1020180000005344

(51) Int. Cl. C07K 16/28; A61K 35/39; C12N 5/071; C07K 16/24; C07K 14/705; A61K 38/51; A61K 38/28; A61P 3/10

(52) U.S. Cl. CPC C07K 16/2863; A61K 35/39; C12N 5/0676; C07K 16/2809; C07K 16/244; C12Y 401/01015; C07K 14/70532; A61K 38/51; A61K 38/28; A61P 3/10; C07K 14/70521

(57) ABSTRACT

The present invention relates to methods of promoting growth of pancreatic islet cells, especially beta islet cells. In particular, the invention relates to methods of promoting growth of pancreatic islet cells by administration of HGF-MET agonists, such as MET agonist antibodies or fragments thereof. The invention further relates to HGF-MET agonists, such as MET agonist antibodies or fragments thereof, and pharmaceutical compositions comprising said agonists, for use in methods of the invention.

### CROSS-REFERENCE TO RELATED APPLICATIONS

This application is a continuation of International Patent Application PCT/EP2019/050084, filed Jan. 3, 2019, which claims the benefit of IT application 1020180000005344, filed Jan. 3, 2018, and is a continuation-in-part of U.S. application Ser. No. 16/313,710, having a filing date of Dec. 27, 2018, which is a 371 application of PCT/EP2017/065599, filed Jun. 23, 2017, and which claims the benefit of GB application 1611123.9, filed Jun. 27, 2016, all of said applications incorporated herein by reference.

### FIELD OF THE INVENTION

The present invention relates to methods of promoting growth of pancreatic islet cells, especially beta islet cells. In particular, the invention relates to methods of promoting growth of pancreatic islet cells by administration of HGF-MET agonists, such as MET agonist antibodies or fragments thereof. The invention further relates to HGF-MET agonists, such as MET agonist antibodies or fragments thereof, and pharmaceutical compositions comprising said agonists, for use in methods of the invention.

### BACKGROUND

The pancreatic islets, or islets of Langerhans, are regions of endocrine tissues and cells situated in the pancreas in so-called “density routes”. Pancreatic islets include alpha, beta, gamma, delta, and epsilon cells, each playing a role in the endocrine activity of the pancreas. In particular, alpha and beta cells are especially important in the regulation of blood glucose levels.

Type 1 diabetes is an autoimmune disease characterized by immune-mediated destruction of pancreatic cells in the islets of Langerhans, especially beta islet cells. This progressive degeneration leads to impairment of insulin production, thus causing high blood glucose levels. Typically, the onset of clinical symptoms is associated with 80-95% reduction in beta cell mass (Klinke, PloS One 3:e1374, 2008). Regenerating beta cells and protecting them from the progressive destruction by immune system is a key unmet medical need in diabetic patients and the Holy Grail in diabetes research.

Although characterized by different etiological mechanisms, type 2 diabetes also leads to Langerhans islet degeneration. In fact, type 2 diabetes is characterized by aberrant insulin production in the presence of insulin resistance, leading to high blood glucose levels and inability of beta cells to compensate for the increased demand of insulin (Christoffersen et al., Am J Physiol Regul Integr Comp Physiol 297:1195-201, 2009). In type 2 diabetes, beta islet cells exhibit defective insulin production and, in late stage disease, the cells themselves can degenerate.

Current management of patients suffering from degeneration of pancreatic islet cells, such as diabetes patients, uses dietary control, with or without administration of insulin. However, this approach does not affect the underlying pathophysiology of the conditions. Novel therapies are therefore needed.

### SUMMARY OF THE INVENTION

It is surprisingly identified herein that MET agonists promote growth of pancreatic islet cells. Moreover, the generated pancreatic islet cells were functional, leading to restoration of insulin production and normalization of glycaemia.

Growth and regeneration of pancreatic islet cells is particularly important in treating diabetes, where the underlying pathophysiology can be treated by the methods described herein. This is a significant improvement on current management of the condition, which simply attempts to control the symptoms.

Promoting growth of pancreatic islet cells is especially important when treating patients in the early stages of type 1 diabetes. Typically, type 1 diabetes symptoms become manifest at adolescence. However, when the pathology is diagnosed, the majority of the patient's pancreatic beta cells have been destroyed (greater than 50%, for example 70% or 80% destruction). Langerhans islet cell degeneration occurs rapidly—as a result, the time-window for effective therapeutic intervention is narrow.

For example, immuno-suppressive drugs are being investigated as therapy for newly-diagnosed type 1 diabetes patients, in an effort to reduce the autoimmune-mediated islet cell destruction. However, immunosuppressants require several months before showing the first clinical benefits. When this occurs, approximately half year after treatment start, the beta cells of the pancreas continue to be destroyed, often completely. As a result, the use of the immunosuppressants is in vain. Maintaining islet (beta) cells during this crucial window is a highly unmet medical need for diabetes patients.

Surprisingly, as demonstrated herein, MET agonists (for example MET agonist antibodies) not only maintain pancreatic islet cell populations, but are able to promote their growth and regeneration. Although animals transgenically overexpressing HGF have been described as exhibiting altered beta cell growth, it was unknown and unclear whether an exogenous, non-native MET-binding agonist would have any effect. It is surprisingly shown herein that administration of a non-native MET agonist can not only maintain islet cell levels in diabetes, but promote their growth and regeneration. Provision of a clinical therapeutic agent able to promote pancreatic islet cell growth has been a long-felt need in diabetes therapy that is solved for the first time herein.

Accordingly, in a first aspect is provided a method of promoting pancreatic islet cell growth comprising administering to a subject an HGF-MET agonist.

It is further shown that HGF-MET agonist promotes insulin-independent uptake of glucose in a model of type I diabetes, and normalises glucose control and overcomes insulin resistance in a model of type II diabetes.

In a further aspect is provided a method of promoting insulin production in a subject exhibiting depressed insulin production, comprising administering to a subject an HGF-MET agonist. In a preferred embodiment, the method is characterised by inducing increased pancreatic islet cell growth.

In a further aspect is provided a method of treating diabetes comprising administering to a subject an HGF-MET agonist. In a preferred embodiment, the method is characterised by inducing increased pancreatic islet cell growth. In another embodiment, the method is characterised by promoting insulin-independent uptake of glucose in a type I diabetes subject. In another embodiment, the method is characterised by overcoming insulin resistance in a type II diabetes subject. In still another embodiment, the method is characterised by treatment of diabetes-associated ulcers and wounds.

In a further aspect is provided an HGF-MET agonist for use in a method provided herein.

In a further aspect is provided a pharmaceutical composition for use in a method provided, wherein the pharmaceutical composition comprises an HGF-MET agonist and a pharmaceutically acceptable excipient or carrier.

In preferred embodiments of all aspects, the HGF-MET agonist is an anti-MET agonist antibody.

### BRIEF DESCRIPTION OF THE DRAWINGS

FIGS. 1A and 1B. MET agonist antibody treatment does not alter basic metabolism in healthy mice. In order to assess the biological effect of a MET agonistic antibody on Langerhans islet cells in vivo, we subjected both male and female adult BALB/c mice to systemic treatment with 0, 3, 10 or 30 mg/kg purified 71D6 antibody for a period of three months (6 mice per gender per group for a total of 48 animals). Antibody was administered 2 times a week by i.p. injection. Body weight and fasting blood glucose concentration was measured every month throughout the experiment. (A) Body weight over time. (B) Basal glycemia over time.

FIG. 2. MET agonist antibody treatment promotes Langerhans islet growth in healthy mice. Adult BALB-c mice were subjected to chronic treatment with increasing concentration of the 71D6 MET agonist antibody as described in FIG. 1 legend. At the end of the experiment, mice were sacrificed and subjected to autopsy. Pancreases were extracted, processed for histological analysis and embedded in paraffin. Sections were stained with hematoxylin and eosin, examined by microscopy and photographed. Images were analysed using ImageJ software to determine Langerhans islet number and size. (A) Mean Langerhans islet density. (B) Mean Langerhans islet size.

FIG. 2C. Representative images of pancreas sections stained with hematoxylin and eosin. Magnification: 400×.

FIG. 3. MET agonist antibody treatment promotes Langerhans islet cell growth in healthy mice. Adult BALB-c mice were subjected to chronic treatment with increasing concentration of the 71D6 MET agonist antibody as described above. Pancreas sections were analysed by immunohistochemistry using anti-insulin antibodies. The Figure shows representative images taken at the microscope at a 100× magnification.

FIG. 4. MET agonist antibodies normalize basal glycaemia in a mouse model of type 1 diabetes mellitus. Streptozotocin (STZ), a chemical agent that selectively kills beta cells and a standard compound used to induce type 1 diabetes mellitus in laboratory animals, was injected i.p. into female BALB-c mice at a dose of 40 mg/kg every 24 hours for 5 consecutive days. One week after the last injection, STZ-treated mice were randomized into 4 arms of 7 mice each based on basal glycemia, which received treatment with (i) vehicle only (STZ), (ii) purified 71D6 antibody (STZ+71D6), (iii) purified 71G2 antibody (STZ+71G2), (iv) purified 71G3 antibody (STZ+71G3). Antibodies were administered by i.p. injection at a dose of 1 mg/kg two times a week for 8 weeks. An additional, fifth arm contained 7 mice that received no STZ or antibody and served as a healthy control (CTRL). Basal glycemia was monitored throughout the experiment. (A) Basal glycaemia over time. (B) Basal glycemia at week 6 of treatment.

FIG. 5. MET agonist antibodies promote Langerhans islet regeneration in a mouse model of type 1 diabetes mellitus. STZ-injected BALB-c mice were treated with 1 mg/kg 71D6, 71G2 or 71G3 as described in FIG. 4 legend. After 8 weeks of antibody treatment, mice were sacrificed and subjected to autopsy. Pancreas sections were stained with hematoxylin and eosin, analysed by microscopy and photographed. Digital images of Langerhans islets were analysed using ImageJ software. The number, density and size of Langerhans islets were determined by digital data analysis. (A) Mean Langerhans islet density. (B) Mean Langerhans islet size.

FIG. 5c. Representative images of pancreas sections stained with hematoxylin and eosin. Magnification: 200×.

FIG. 6. MET agonist antibodies promote pancreatic islet cell regeneration in a mouse model of type 1 diabetes mellitus. STZ-injected BALB-c mice were treated with 1 mg/kg 71D6, 71G2 or 71G3 as described above. Pancreas sections were analysed by immunohistochemistry using anti-insulin antibodies. The Figure shows representative images taken at the microscope at a 200× magnification.

FIG. 7. MET agonist antibodies normalize basal glycaemia in a mouse model of type 2 diabetes mellitus. Female db/db mice were randomized into 4 arms of five mice each, which received treatment with (i) vehicle only (PBS), (ii) purified 71D6 antibody, (iii) purified 71G2 antibody, (iv) purified 71G3 antibody. Antibodies were administered by i.p. injection at a dose of 1 mg/kg two times a week for 8 weeks. C57BL6/J mice were used as non-diabetic control animals. Basal glycemia was monitored throughout the experiment. (A) Basal glycaemia over time. (B) Basal glycemia at week 8 of treatment.

FIG. 8. MET agonist antibodies promote Langerhans islet regeneration in a mouse model of type 2 diabetes mellitus. Female db/db mice were treated with 71D6, 71G2 and 71G3 as described in FIG. 7 legend. After 8 weeks of treatment, mice were sacrificed and subjected to autopsy. Pancreases were collected, processed for histology and embedded in paraffin. Tissues sections were stained with hematoxylin and eosin, analysed by microscopy, and photographed. Langerhans islets were analysed using ImageJ software to estimate islet number, density and size. (A) Mean Langerhans islet density. (B) Mean Langerhans islet size.

FIG. 8c. Representative images of pancreas sections stained with hematoxylin and eosin. Magnification: 200×.

FIG. 9. MET agonist antibodies promote pancreatic islet cell regeneration in a mouse model of type 2 diabetes mellitus. Female db/db mice were treated with 71D6, 71G2 and 71G3 as described above. Pancreas sections were analysed by immunohistochemistry using anti-insulin antibodies. The Figure shows representative images taken at the microscope at a 100× magnification.

FIG. 10. Blood sugar content in NOD mice. Blood sugar was measured in random fed (i.e. not fasting) animals using test strips for human use (multiCare in; Biochemical Systems International). At week 6 of age, NOD mice displayed a pre-diabetic, average glycemia of approximately 110 mg/dL. Starting from week 7, animals were subjected to treatment as described in the text. Glycemia was monitored one time per week for the whole duration of the experiment. An animal was considered diabetic if it showed a glycemic value greater than 250 mg/dL (horizontal dotted lines) for 2 consecutive weeks.

FIG. 11. Analysis of diabetes onset. (A) Percentage of diabetic mice overtime. The vertical dotted line indicates the time of treatment start. (B) Kaplan-Meier analysis of diabetes onset. Statistical analysis was performed using Prism software (Graph Pad). A Mantel-Cox test, a Logrank test for trend and a Gehan-Breslow-Wilcoxon test all gave a p value of less than 0.001, indicating that the differences among curves are statistically significant.

FIG. 12. Analysis of non-fasting glycemia over time. Glycemia was measured in random fed (i.e. not fasting) animals as described above one time per week. Consistent with the diabetes onset data, blood sugar levels followed a precise order CONTROL>CD3>71D6>COMBO.

FIG. 13. Glucose tolerance test (GTT). Before sacrifice, all mice were subjected to a glucose tolerance test (GTT). To this end, animals were food-starved overnight. The morning after, a blood sample was collected for glycemia and insulin measurement. A glucose solution (3 g/kg in 200 μL PBS) was injected i.p. and a second blood sample was collected 3 minutes later. Blood glucose concentration was determined using strips as described above. Insulin concentration was measured with an Ultra-Sensitive Mouse Insulin ELISA Kit (Crystal Chem). (A) Glycemia at time zero. (B) Glycemia at time 3 minutes. (C) Insulin levels at time zero. (D) Insulin levels at time 3 minutes.

FIG. 14. Body weight and liver to body weight at autopsy. (A) Body weight. Consistent with an ameliorated diabetic phenotype, body weight was slightly (although not significantly) higher in the treatment arms compared to the control arm. (B) Liver to body weight. There was no significant difference in liver to body weight in any of the group, suggesting that 71D6-mediated liver growth (observed in other mouse systems) is strain-specific.

FIG. 15. Histological analysis of pancreas sections. Pancreas samples were embedded in paraffin and processed for histological analysis. Tissue section were stained with hematoxylin and eosin (H&E) and analyzed by microscopy. Representative images of each treatment arm are shown. Magnification: 200×.

FIG. 16. Immuno-histochemical analysis of Insulin expression. Pancreas sections were stained with anti-insulin antibodies and analyzed by microscopy. Representative images of each treatment arm are shown. Magnification: 40×.

FIG. 17. High power microscopical analysis of Insulin expression. Pancreas sections were stained with anti-insulin antibodies as above. Representative microscope images of each treatment arm are shown. Magnification: 200×.

FIG. 18. Anti-insulin auto-antibodies in mouse plasma. Plasma samples collected at autopsy from all mice as well as from young, pre-diabetic female NOD mice (week 7 of life) were analyzed using a Mouse IAA (Insulin Auto-Antibodies) ELISA Kit (Fine Test). This analysis revealed that most mice displayed high concentrations of anti-insulin antibodies compared to pre-diabetic animals (last group on the right). While no statistically significant difference was observed among the different populations, mice of the COMBO arm displayed a trend towards lower levels. Mice of the 71D6 arm could be clearly divided into 2 subpopulations with low and high auto-antibodies levels, respectively. While these results warrant further investigation, they overall strengthen the hypothesis that neither anti-CD3 antibodies nor 71D6 treatment affect the production of auto-antibodies in this system, but rather act downstream to prevent or delay the onset of diabetes.

FIG. 19. Type I diabetes model: promotion of glucose uptake and cooperation with Insulin in diabetic mice. Pancreatic β-cell degeneration was induced in BALB/c mice by i.p. injection of streptozotocin (STZ). STZ-treated mice displayed a mean basal glycemy two times higher compared to untreated mice. STZ-treated mice were randomized into 4 arms, which received treatment with 71G3, 71D6, 71G2 or vehicle only (PBS), respectively. An additional, fifth control arm received no STZ or antibody and served as healthy control. Blood glucose concentration in fasting conditions was monitored over time for 5 weeks. At the end of week 5, a glucose tolerance test (GTT) and an insulin tolerance test (ITT) were performed. (A) Analysis over time of basal blood glucose levels in fasting conditions. (B) GTT: following oral administration of glucose to a fasting animal, blood glucose levels are monitored over time. (C) ITT: following i.p. injection of insulin to a partially fasting animal, blood glucose levels are monitored overtime.

FIG. 20. Type I diabetes model: promotion of glucose uptake and cooperation with Insulin in cultured cells. C2C12 mouse myoblast cells were induced to differentiate into myocytes and then incubated with human/mouse equivalent agonistic anti-MET antibodies (71G3, 71D6, 71G2). After 24 hours, antibody-treated cells were divided into 3 arms, which were subjected to acute stimulation with 0 nM, 100 nM or 1000 nM human recombinant insulin for 1 hour in the presence of the fluorescent glucose analogue 2-(N-(7-Nitrobenz-2-oxa-1,3-diazol-4-yl)Amino)-2-Deoxyglucose (2-NBDG). 2-NBDG uptake was determined by flow cytometry. (A) Induction of 2-NBDG uptake by human/mouse equivalent agonistic anti-MET antibodies or insulin. (B) Induction of 2-NBDG uptake by 71G3 in the absence or presence of insulin. (C) Induction of 2-NBDG uptake by 71D6 in the absence or presence of insulin. (D) Induction of 2-NBDG uptake by 71G2 in the absence or presence of insulin.

FIG. 21. Type II diabetes model: blood glucose level normalization and insulin resistance overcoming in db/db mice. At the age of 8 weeks, female db/db mice (a C57BLKS/J variant bearing a point mutation in the leptin receptor gene lepr) were randomized into 4 arms that received treatment with 71G3, 71D6, 71G2 or vehicle only (PBS), respectively. Antibodies were administered two times a week by i.p. injection at a dose of 1 mg/kg. Blood glucose concentration in fasting conditions was monitored every 10 days for 7 weeks. At the end of the treatment, i.e. when mice were 15 weeks old, a glucose tolerance test (GTT) and an insulin tolerance test (ITT) were performed using age-matched wild-type C57BLKS/J mice as control. (A) Blood glucose concentration over time. (B) GTT: following oral administration of glucose to a fasting animal, blood glucose levels are monitored over time. (C) ITT: following i.p. injection of insulin to a partially fasting animal, blood glucose levels are monitored overtime.

FIG. 22. Mouse model of diabetic ulcers: accelerated healing of wounds. Eight week-old db/db diabetic mice were subjected to anaesthesia and then cut with a 0.8 cm-wide circular punch blade for skin biopsies to create a round wound in the right posterior flank. The entire epidermal layer was removed. The day after surgery, mice were randomized into 4 arms that received treatment with purified 71G3, 71D6 and 71G2 or vehicle only (PBS). Antibodies were delivered every second day by i.p injection at a dose of 5 mg/kg. Wound diameter was measured every day using a calliper. (A) Wound area over time. (B) Mean re-epithelization rate as determined by averaging the daily % of wound closure.

FIG. 23. Comparison with prior art antibodies: human-mouse cross-reactivity. Human or mouse MET ECD was immobilized in solid phase and exposed to increasing concentrations of antibodies (all in a mouse IgG/λ format) in solution. Binding was revealed by ELISA using HRP-conjugated anti-mouse Fc antibodies.

FIG. 24. Comparison with prior art antibodies: MET auto-phosphoryation. A549 human lung carcinoma cells and MLP29 mouse liver precursor cells were deprived of serum growth factors for 48 hours and then stimulated with increasing concentrations of antibodies. After 15 minutes of stimulation, cells were lysed, and phospho-MET levels were determined by ELISA using anti-MET antibodies for capture and anti-phospho-tyrosine antibodies for revealing.

FIG. 25. Comparison with prior art antibodies: branching morphogenesis. LOC human kidney epithelial cell spheroids were seeded in a collagen layer and then incubated with increasing concentrations of mAbs. Branching morphogenesis was followed over time by microscopy, and colonies were photographed after 5 days.

FIG. 26. Comparison with prior art antibodies: branching morphogenesis. MLP29 mouse liver precursor cell spheroids were seeded in a collagen layer and then incubated with increasing concentrations of mAbs. Branching morphogenesis was followed over time by microscopy, and colonies were photographed after 5 days.

### DETAILED DESCRIPTION OF THE INVENTION

As used herein, “pancreatic islet cell” is used to refer to those islet cells of the pancreas also known as “islets of Langerhans”, and include alpha, beta, and delta islet cells, plus islet stroma. Means of identifying pancreatic islet cells are known to the skilled person, for example histological examination of cell biopsies.

Promotion of islet cell growth as used herein can refer to an increase in the growth of pancreatic islet cells in a subject that has received an HGF-MET agonist compared to in that subject prior to intervention. Similarly, promotion of islet cell growth can refer to an increase of pancreatic islet cells in a subject that has received an HGF-MET agonist compared to a comparable control subject that has not received an HGF-MET agonist. Pancreatic islet cell growth can be characterised by an increase in the density of islets (number per mm²), an increase in the islet size (e.g. area), or both an increase in islet density and islet size.

Promotion of beta islet cell growth as used herein can refer to an increase in the growth of beta islet cells in a subject that has received an HGF-MET agonist compared to in that subject prior to intervention. Similarly, promotion of beta islet cell growth can refer to an increase of pancreatic islet cells in a subject that has received an HGF-MET agonist compared to a comparable control subject that has not received an HGF-MET agonist. Pancreatic islet cell growth can be characterised by an increase in the density of islets (number per mm²), an increase in the islet size (e.g. area), or both an increase in islet density and islet size.

Promotion of insulin production as used herein can refer to an increase in the insulin production by (beta) islet cells in a subject that has received an HGF-MET agonist compared to in that subject prior to intervention. Similarly, promotion of insulin production can refer to an increase of insulin production by (beta) islet cells in a subject that has received an HGF-MET agonist compared to a comparable control subject that has not received an HGF-MET agonist. Insulin production can be characterised by one or more of an increase in plasma insulin levels, an increase in beta cell density, an increase in beta cell area, an increase in density and/or number of insulin-positive islet cells, or any combination of these measures.

A pancreatic tissue transplant, as used herein, refers to the transplant of any pancreatic tissue into a subject. The transplant may be a whole organ transplant—i.e. a whole pancreas transplant—or a partial pancreas transplant. The transplant may be a transplant of pancreatic islets or islet cells, also referred to herein as an pancreatic islet graft.

As used herein, “HGF-MET agonist” and “MET agonist” are used interchangeably to refer to non-native agents that promote signalling via the MET protein—i.e. agents other than HGF that bind MET and increase MET signalling. Agonist activity on binding of MET by MET agonists is indicated by molecular and/or cellular responses that (at least partially) mimic the molecular and cellular responses induced upon HGF-MET binding. Suitable methods for measuring MET agonist activity are described herein, including in the Examples. A“full agonist” is a MET agonist that increases MET signalling in response to binding to an extent at least similar, and optionally exceeding, the extent to which MET signalling increases in response to binding of the native HGF ligand. Examples of the level of MET signalling induced by “full agonists”, as measured by different methods of determining MET signalling, are provided herein.

Immunosuppressive agents, also referred to as immunosuppressants, as used herein refer to therapeutic agents intended to reduce or inhibit an immune response in a subject, for example anti-inflammatory agents and tolerising agents. Examples of immunosuppressants include check-point inhibitors (e.g. PD-L1 molecules, CTLA4 molecules (e.g. abatecept)), TNF inhibitors (e.g. anti-TNF antibodies, etanercept), tolerising dendritic cells, anti-CD3 antibodies, anti-inflammatory cytokines (e.g. IL-10).

HGF-MET agonists may be small molecules, binding proteins such as antibodies or antigen binding fragments, aptamers or fusion proteins. A particular example of a MET agonist is an anti-MET agonist antibody.

As used herein, “treatment” or “treating” refers to effective therapy of the relevant condition—that is, an improvement in the health of the subject. Treatment may be therapeutic or prophylactic treatment—that is, therapeutic treatment of subjects suffering from the condition, or prophylactic treatment of a subject so as to reduce their risk of contracting the condition or the severity of the condition once contracted. Therapeutic treatment may be characterised by improvement in the health of the subject compared to prior to treatment. Therapeutic treatment may be characterised by improvement in the health of the subject compared to a comparable control subject that has not received treatment. Therapeutic treatment may also be characterised by stabilisation of the health of the subject compared to prior to treatment, i.e. inhibition of progression of a disease state in the subject. Prophylactic treatment may be characterised by improvement in the health of the subject compared to a control subject (or population of control subjects) that has not been treated.

As used herein, the term “antibody” includes an immunoglobulin having a combination of two heavy and two light chains which have significant specific immuno-reactive activity to an antigen of interest (e.g. human MET). The terms “anti-MET antibodies” or “MET antibodies” are used interchangeably herein to refer to antibodies which exhibit immunological specificity for human MET protein. “Specificity” for human MET does not exclude cross-reaction with species homologues of MET. In particular, “agomAbs” as used herein refers MET antibodies that bind to both human MET and mouse MET.

“Antibody” as used herein encompasses antibodies of any human class (e.g. IgG, IgM, IgA, IgD, IgE) as well as subclasses/isotypes thereof (e.g. IgG1, IgG2, IgG3, IgG4, IgA1). Antibody as used herein also refers to modified antibodies. Modified antibodies include synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecifc, etc.) altered to bind to two or more different antigens or to different epitopes on a single antigen); heavy chain molecules joined to scFv molecules and the like. In addition, the term “modified antibody” includes multivalent forms of antibodies (e.g., trivalent, tetravalent, etc., antibodies that bind to three or more copies of the same antigen).

Antibodies described herein may possess antibody effector function, for example one or more of antibody dependent cell-mediated cytotoxicity (ADCC), complement dependent cytotoxicity (CDC) and antibody dependent cellular phagocytosis (ADCP). Alternatively, in certain embodiments antibodies for use according to the invention have an Fc region that has been modified such that one or more effector functions, for example all effector functions, are abrogated.

Antibodies comprise light and heavy chains, with or without an interchain covalent linkage between them. An antigen-binding fragment of an antibody includes peptide fragments that exhibit specific immuno-reactive activity to the same antigen as the antibody (e.g. MET). Examples of antigen-binding fragments include scFv fragments, Fab fragments, and F(ab′)2 fragments.

As used herein, the terms “variable region” and “variable domain” are used interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20, 267-279, 2000). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20, 267-279, 2000). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20, 267-279, 2000).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, Md., 1991) and the limits of the HVs and the CDRs may be different in some VH and VL domains.

The CDRs of the VL and VH domains can typically be defined as comprising the following amino acids: residues 24-34 (CDRL1), 50-56 (CDRL2) and 89-97 (CDRL3) in the light chain variable domain, and residues 31-35 or 31-35b (CDRH1), 50-65 (CDRH2) and 95-102 (CDRH3) in the heavy chain variable domain; (Kabat et al., Sequences of Proteins of Immunological Interest, 5th Ed. Public Health Service, National Institutes of Health, Bethesda, Md., 1991). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227, 799-817, 1992; Tramontano et al., J. Mol. Biol, 215, 175-182, 1990). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “CDR” or “complementarity determining region” means the non-contiguous antigen combining sites found within the variable region of both heavy and light chain polypeptides. These particular regions have been described by Kabat et al., J. Biol. Chem. 252, 6609-6616, 1977, by Kabat et al., Sequences of Proteins of Immunological Interest, 5th Ed. Public Health Service, National Institutes of Health, Bethesda, Md., 1991, by Chothia et al., J. Mol. Biol. 196, 901-917, 1987, and by MacCallum et al., J. Mol. Biol. 262, 732-745, 1996, where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions.

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework region” or “FR region” includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161, 4083-4090, 1998). MET antibodies comprising a“fully human” hinge region may contain one of the hinge region sequences shown in Table 2 below.

TABLE 2
Human hinge sequences.

| IgG | Upper hinge | Middle hinge | Lower hinge |
|---|---|---|---|
| IgG1 | EPKSCDKTHT | CPPCP | APELLGGP |
| | (SEQ ID NO: 199) | (SEQ ID NO: 200) | (SEQ ID NO: 201) |
| IgG3 | ELKTPLGDTTHT | CPRCP(EPKSCDTPPPCPRCP)₃ | APELLGGP |
| | (SEQ ID NO: 202) | (SEQ ID NO: 203) | (SEQ ID NO: 204) |
| IgG4 | ESKYGPP | CPSCP | APEFLGGP |
| | (SEQ ID NO: 205) | (SEQ ID NO: 206) | (SEQ ID NO: 207) |
| IgG42 | ERK | CCVECPPPCP | APPVAGP |
| | (SEQ ID NO: 208) | (SEQ ID NO: 209) | (SEQ ID NO: 210) |

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; Kabat at al., Sequences of Proteins of Immunological Interest, 5th Ed. Public Health Service, National Institutes of Health, Bethesda, Md. (1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “fragment” refers to a part or portion of an antibody or antibody chain comprising fewer amino acid residues than an intact or complete antibody or antibody chain. The term “antigen-binding fragment” refers to a polypeptide fragment of an immunoglobulin or antibody that binds antigen or competes with intact antibody (i.e., with the intact antibody from which they were derived) for antigen binding (i.e., specific binding to MET). As used herein, the term “fragment” of an antibody molecule includes antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen). The subject binding molecules have at least one binding site specific for hMET.

As used herein, the term “specificity” refers to the ability to bind (e.g., immunoreact with) a given target, e.g., hMET, mMET. A poypeptide may be monospecific and contain one or more binding sites which specifically bind a target or a poypeptide may be multispecific and contain two or more binding sites which specifically bind the same or different targets. In one embodiment, an antibody of the invention is specific for more than one target. For example, in one embodiment, a multispecific binding molecule of the invention binds hMET and a second target molecule. In this context, the second target molecule is a molecule other than hMET or mMET.

The term “epitope” refers to the portion(s) of an antigen (e.g. human MET) that contact an antibody. Epitopes can be linear, i.e., involving binding to a single sequence of amino acids, or conformational, i.e., involving binding to two or more sequences of amino acids in various regions of the antigen that may not necessarily be contiguous. The antibodies provided herein may bind to different (overlapping or non-overlapping) epitopes within the extracellular domain of the human MET protein.

As used herein the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life or effector function.

As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain portion lacking a CH2 domain and comprising a binding domain of a poypeptide comprising the binding portion of one member of a receptor ligand pair.

The term “modified antibody” may also be used herein to refer to amino acid sequence variants of a MET antibody of the invention. It will be understood by one of ordinary skill in the art that a MET antibody of the invention may be modified to produce a variant MET antibody which varies in amino acid sequence in comparison to the MET antibody from which it was derived. For example, nucleotide or amino acid substitutions leading to conservative substitutions or changes at “non-essential” amino acid residues may be made (e.g., in CDR and/or framework residues). Amino acid substitutions can include replacement of one or more amino acids with a naturally occurring or non-natural amino acid.

As used herein, the term “humanising substitutions” refers to amino acid substitutions in which the amino acid residue present at a particular position in the VH or VL domain of a MET antibody of the invention (for example a camelid-derived MET antibody) is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of a MET antibody, defined herein.

As used herein the term “humanised variant” refers to a variant antibody which contains one or more “humanising substitutions” compared to a reference MET antibody, wherein a portion of the reference antibody (e.g. the VH domain and/or the VL domain or parts thereof containing at least one CDR) has an amino acid sequence derived from a non-human species, and the “humanising substitutions” occur within the amino acid sequence derived from a non-human species.

The term “germlined variant” is used herein to refer specifically to “humanised variants” in which the “humanising substitutions” result in replacement of one or more amino acid residues present at a particular position (s) in the VH or VL domain of a MET antibody of the invention (for example a camelid-derived MET antibody) with an amino acid residue which occurs at an equivalent position in a reference human VH or VL domain encoded by the human germline. It is typical that for any given “germlined variant”, the replacement amino acid residues substituted into the germlined variant are taken exclusively, or predominantly, from a single human germline-encoded VH or VL domain. The terms “humanised variant” and “germlined variant” are often used interchangeably herein. Introduction of one or more “humanising substitutions” into a camelid-derived (e.g. llama derived) VH or VL domain results in production of a “humanised variant” of the camelid (llama)-derived VH or VL domain. If the amino acid residues substituted in are derived predominantly or exclusively from a single human germline-encoded VH or VL domain sequence, then the result may be a “human germlined variant” of the camelid (llama)-derived VH or VL domain.

As used herein, the term “affinity variant” refers to a variant antibody which exhibits one or more changes in amino acid sequence compared to a reference MET antibody of the invention, wherein the affinity variant exhibits an altered affinity for hMET and/or mMET in comparison to the reference antibody. Preferably the affinity variant will exhibit improved affinity for hMET and/or mMET, as compared to the reference MET antibody. The improvement may be apparent as a lower KD for hMET and/or for mMET, or a slower off-rate for hMET and/or for mMET. Affinity variants typically exhibit one or more changes in amino acid sequence in the CDRs, as compared to the reference MET antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

As used herein, antibodies having “high human homology” refers to antibodies comprising a heavy chain variable domain (VH) and a light chain variable domain (VL) which, taken together, exhibit at least 90% amino acid sequence identity to the closest matching human germline VH and VL sequences. Antibodies having high human homology may include antibodies comprising VH and VL domains of native non-human antibodies which exhibit sufficiently high % sequence identity to human germline sequences, including for example antibodies comprising VH and VL domains of camelid conventional antibodies, as well as engineered, especially humanised or germlined, variants of such antibodies and also “fully human” antibodies.

In one embodiment the VH domain of the antibody with high human homology may exhibit an amino acid sequence identity or sequence homology of 80% or greater with one or more human VH domains across the framework regions FR1, FR2, FR3 and FR4. In other embodiments the amino acid sequence identity or sequence homology between the VH domain of the polypeptide of the invention and the closest matching human germline VH domain sequence may be 85% or greater, 90% or greater, 95% or greater, 97% or greater, or up to 99% or even 100%.

In one embodiment the VH domain of the antibody with high human homology may contain one or more (e.g. 1 to 10) amino acid sequence mis-matches across the framework regions FR1, FR2, FR3 and FR4, in comparison to the closest matched human VH sequence.

In another embodiment the VL domain of the antibody with high human homology may exhibit a sequence identity or sequence homology of 80% or greater with one or more human VL domains across the framework regions FR1, FR2, FR3 and FR4. In other embodiments the amino acid sequence identity or sequence homology between the VL domain of the polypeptide of the invention and the closest matching human germline VL domain sequence may be 85% or greater 90% or greater, 95% or greater, 97% or greater, or up to 99% or even 100%.

In one embodiment the VL domain of the antibody with high human homology may contain one or more (e.g. 1 to 10) amino acid sequence mis-matches across the framework regions FR1, FR2, FR3 and FR4, in comparison to the closest matched human VL sequence.

Before analysing the percentage sequence identity between the antibody with high human homology and human germline VH and VL, the canonical folds may be determined, which allows the identification of the family of human germline segments with the identical combination of canonical folds for H1 and H2 or L1 and L2 (and L3). Subsequently the human germline family member that has the highest degree of sequence homology with the variable region of the antibody of interest is chosen for scoring the sequence homology. Procedures for determining the closest matching human germline, and determining % sequence identity/homology, are well-known to the skilled person.

Antibodies with high human homology may comprise hypervariable loops or CDRs having human or human-like canonical fold structures. In one embodiment at least one hypervariable loop or CDR in either the VH domain or the VL domain of the antibody with high human homology may be obtained or derived from a VH or VL domain of a non-human antibody, for example a conventional antibody from a species of Camelidae, yet exhibit a predicted or actual canonical fold structure which is substantially identical to a canonical fold structure which occurs in human antibodies. In one embodiment, both H1 and H2 in the VH domain of the antibody with high human homology exhibit a predicted or actual canonical fold structure which is substantially identical to a canonical fold structure which occurs in human antibodies.

Antibodies with high human homology may comprise a VH domain in which the hypervariable loops H1 and H2 form a combination of canonical fold structures which is identical to a combination of canonical structures known to occur in at least one human germline VH domain. It has been observed that only certain combinations of canonical fold structures at H1 and H2 actually occur in VH domains encoded by the human germline. In an embodiment H1 and H2 in the VH domain of the antibody with high human homology may be obtained from a VH domain of a non-human species, e.g. a Camelidae species, yet form a combination of predicted or actual canonical fold structures which is identical to a combination of canonical fold structures known to occur in a human germline or somatically mutated VH domain. In non-limiting embodiments H1 and H2 in the VH domain of the antibody with high human homology may be obtained from a VH domain of a non-human species, e.g. a Camelidae species, and form one of the following canonical fold combinations: 1-1, 1-2, 1-3, 1-6, 1-4, 2-1, 3-1 and 3-5. An antibody with high human homology may contain a VH domain which exhibits both high sequence identity/sequence homology with human VH, and which contains hypervariable loops exhibiting structural homology with human VH.

It may be advantageous for the canonical folds present at H1 and H2 in the VH domain of the antibody with high human homology, and the combination thereof, to be “correct” for the human VH germline sequence which represents the closest match with the VH domain of the antibody with high human homology in terms of overall primary amino acid sequence identity. By way of example, if the closest sequence match is with a human germline VH3 domain, then it may be advantageous for H1 and H2 to form a combination of canonical folds which also occurs naturally in a human VH3 domain. This may be particularly important in the case of antibodies with high human homology which are derived from non-human species, e.g. antibodies containing VH and VL domains which are derived from camelid conventional antibodies, especially antibodies containing humanised camelid VH and VL domains.

Thus, in one embodiment the VH domain of the MET antibody with high human homology may exhibit a sequence identity or sequence homology of 80% or greater, 85% or greater, 90% or greater, 95% or greater, 97% or greater, or up to 99% or even 100% with a human VH domain across the framework regions FR1, FR2, FR3 and FR4, and in addition H1 and H2 in the same antibody are obtained from a non-human VH domain (e.g. derived from a Camelidae species, preferably llama), but form a combination of predicted or actual canonical fold structures which is the same as a canonical fold combination known to occur naturally in the same human VH domain.

In other embodiments, L1 and L2 in the VL domain of the antibody with high human homology are each obtained from a VL domain of a non-human species (e.g. a camelid-derived VL domain), and each exhibits a predicted or actual canonical fold structure which is substantially identical to a canonical fold structure which occurs in human antibodies.

L1 and L2 in the VL domain of an antibody with high human homology may form a combination of predicted or actual canonical fold structures which is identical to a combination of canonical fold structures known to occur in a human germline VL domain. In non-limiting embodiments L1 and L2 in the VLambda domain of an antibody with high human homology (e.g. an antibody containing a camelid-derived VL domain or a humanised variant thereof) may form one of the following canonical fold combinations: 11-7, 13-7(A,B,C), 14-7(A,B), 12-11, 14-11 and 12-12 (as defined in Williams et al., J. Mol. Biol. 264, 220-232, 1996, and as shown on http://www.bioc.uzh.ch/antibody/Sequences/Germlines/VBase_hVL.html). In non-limiting embodiments L1 and L2 in the Vkappa domain may form one of the following canonical fold combinations: 2-1, 3-1, 4-1 and 6-1 (as defined in Tomlinson et al., EMBO J. 14, 4628-4638, 1995 and as shown on http://www.bioc.uzh.ch/antibody/Sequences/Germlines/VBase_hVK.html). In a further embodiment, all three of L1, L2 and L3 in the VL domain of an antibody with high human homology may exhibit a substantially human structure. It is preferred that the VL domain of the antibody with high human homology exhibits both high sequence identity/sequence homology with human VL, and also that the hypervariable loops in the VL domain exhibit structural homology with human VL.

In one embodiment, the VL domain of a MET antibody with high human homology may exhibit a sequence identity of 80% or greater, 85% or greater, 90% or greater, 95% or greater, 97% or greater, or up to 99% or even 100% with a human VL domain across the framework regions FR1, FR2, FR3 and FR4, and in addition hypervariable loop L1 and hypervariable loop L2 may form a combination of predicted or actual canonical fold structures which is the same as a canonical fold combination known to occur naturally in the same human VL domain.

It is, of course, envisaged that VH domains exhibiting high sequence identity/sequence homology with human VH, and also structural homology with hypervariable loops of human VH will be combined with VL domains exhibiting high sequence identity/sequence homology with human VL, and also structural homology with hypervariable loops of human VL to provide antibodies with high human homology containing VH/VL pairings (e.g. camelid-derived VH/VL pairings) with maximal sequence and structural homology to human-encoded VH/VL pairings.

Procedures for evaluating camelid-derived (e.g. llama-derived) CDRs, VH domains or VL domains for the presence of human-like canonical fold structures are described in WO 2010/001251 and WO 2011/080350, the contents of which are incorporated herein in their entirety by reference.

As used herein, the term “affinity” or “binding affinity” should be understood based on the usual meaning in the art in the context of antibody binding, and reflects the strength and/or stability of binding between an antigen and a binding site on an antibody or antigen binding fragment thereof. As used herein, “subject” and “patient” are used interchangeably to refer to a human individual. A“control subject” refers to a comparable subject that has not received the intervention.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Therapeutic Methods

It is demonstrated herein that HGF-MET agonists (especially MET agonist antibodies) promote growth of pancreatic islet cells in healthy subjects. It is also demonstrated that MET agonists (especially MET agonist antibodies) protect pancreatic islet cells from degeneration in subjects experiencing islet cell depletion or damage. Moreover, not only do HGF-MET agonists (especially MET agonist antibodies) protect islet cells in these subjects, but they promote growth and regeneration of new islet cells in subjects with depleted or degenerated pancreatic islet cell populations. Furthermore, the new islet cells induced by MET agonist administration are highly functional, restoring insulin production.

Promoting islet cell growth is particularly advantageous, as it treats the underlying pathophysiology of conditions such as diabetes (especially type 1 diabetes, but also type 2 diabetes). Current treatment relies on passively managing the symptoms using diet and often insulin injections. These approaches do not address the underlying cause of the disease. Herein it is surprisingly identified that administration of an exogenous, non-native HGF-MET agonist effectively promotes growth and regeneration of pancreatic islet cells. Therefore administration of an HGF-MET agonist (especially a MET agonist antibody) represents a solution to the long felt medical need for a clinically relevant therapy that addresses the problem of pancreatic cell degradation.

Accordingly, in one aspect, provided herein is a method of promoting pancreatic islet cell growth comprising administering to a subject an HGF-MET agonist. Also provided is an HGF-MET agonist for use for promoting pancreatic islet cell growth in a subject, or the use of an HGF-MET agonist for the manufacture of a medicament for promoting pancreatic islet cell growth in a subject.

In a further aspect is provided a method of promoting insulin production in a subject in need thereof, comprising administering to a subject an HGF-MET agonist. In a preferred embodiment of this aspect, the method is characterised by inducing increased pancreatic islet cell growth. Also provided is an HGF-MET agonist for use for promoting insulin production in a subject, or the use of an HGF-MET agonist for the manufacture of a medicament for promoting insulin production in a subject.

It is further demonstrated herein that administration of HGF-MET agonists (MET antibodies) which induce MET signalling is able to restore metabolic function in diabetes, including both type I and type II diabetes. In particular, in a model of type I diabetes, MET antibodies are shown to promote glucose uptake. Furthermore, administration of MET antibodies with insulin resulted in a synergistic effect on glucose uptake. In a model of type II diabetes, MET antibodies are shown to normalise glucose control and to reduce insulin resistance.

In a further aspect is provided method of treating diabetes comprising administering to a subject an HGF-MET agonist. In a preferred embodiment of this aspect, the method is characterised by inducing increased pancreatic islet cell growth. Alternatively, or in addition, the method is further characterised by promoting insulin production. In a further aspect is provided an HGF-MET agonist (for example a MET agonist antibody) for use in a method of treating diabetes, wherein the HGF-MET agonist promotes pancreatic islet cell growth. In still a further aspect is provided an HGF-MET agonist for use in a method of treating diabetes, wherein the HGF-MET agonist promotes insulin production. Also provided is an HGF-MET agonist for use for treating diabetes in a subject, or the use of an HGF-MET agonist for the manufacture of a medicament for treating diabetes in a subject.

As demonstrated herein, HGF-MET agonists (in particular MET agonist antibodies) promote pancreatic islet cell growth. This growth is characterised both by an increase in pancreatic islet cell area, as well as an increase in the density of islets in pancreatic tissue.

Accordingly, in a preferred embodiment of all methods provided herein, the method increases pancreatic islet cell density. In a preferred embodiment of all methods provided herein, the method increases pancreatic islet cell area.

It is demonstrated herein that HGF-MET agonists (e.g. MET agonist antibodies) promote growth of all pancreatic islet cells—that is, alpha, beta, gamma, delta and epsilon cells. Accordingly, in certain embodiments of all methods provided herein, the method promotes growth of any one or more of: alpha cells, beta cells, gamma cells, delta cells and epsilon cells. In certain embodiments, the method promotes growth of alpha cells. In certain embodiments, the method promotes growth of beta cells. In certain embodiments, the method promotes growth of gamma cells. In certain embodiments, the method promotes growth of delta cells. In certain embodiments, the method promotes growth of epsilon cells.

It is further demonstrated herein that HGF-MET agonists (e.g. MET agonist antibodies) are particularly effective at promoting growth of beta islet cells. This is particularly advantageous, as beta cells are crucial for insulin production and effective glucose control, and are degraded in conditions such as diabetes. Not only do HGF-MET agonists (e.g. MET agonist antibodies) promote beta cell growth, but the new cells are highly functional and produce insulin.

Accordingly, in preferred embodiments of all methods provided herein, the method promotes beta islet cell growth. In a preferred embodiment, the method increases beta islet cell density. In a preferred embodiment, the method increases beta islet cell area. In a preferred embodiment, the method promotes growth of insulin-producing beta cells.

The methods described herein will also be particularly advantageous in subjects that receive a pancreatic tissue transplant. Pancreatic tissue transplant is a possible treatment in subjects (such as diabetic subjects) where the islet cells have been destroyed. Such transplants can be in the form of a whole pancreas transplant, partial transplant of portion of a pancreas, or graft of isolated islets. In all instances, methods provided herein will be particularly advantageous in patients receiving such transplants and grafts, since the methods will promote survival of the transplanted islets and also growth and expansion of those cells.

Accordingly, in embodiments of all methods provided herein, the method further comprises administering to the subject a pancreatic tissue transplant. In certain embodiments, the method further comprises administering to the subject a whole pancreas transplant. In certain embodiments, the method further comprises administering to the subject a partial pancreas transplant. In certain embodiments, the method further comprises administering to the subject a pancreatic islet graft. In all such embodiments, administration of the HGF-MET agonist (for example a MET agonist antibody) and administration of the transplant can be performed in any order, or simultaneously.

In a further aspect is provided a method of improving pancreatic tissue transplant in a subject in need thereof, the method comprising administering to the subject an HGF-MET agonist. Also provided is an HGF-MET agonist for improving pancreatic tissue transplant in a subject, or the use of an HGF-MET agonist for the manufacture of a medicament for improving pancreatic tissue transplant in a subject. By “improving pancreatic tissue transplant” it is herein meant that graft survival following transplantation and following proliferation of engrafted cells or tissue are improved.

Administration of HGF-MET agonists (e.g. MET agonist antibodies) is particularly advantageous in a type 1 diabetes context. Type 1 diabetes is characterised by significant, and often complete, degradation of the subject's beta islet cells. As a result, the subject cannot produce insulin and therefore cannot manage their blood glucose properly. As demonstrated herein, administration of HGF-MET agonists (e.g. MET agonist antibodies) can promote pancreatic islet cells (especially beta cells) even in subjects with depleted islet cell populations. These new islet cells as a result of the methods provided herein are functional, producing insulin. Type 1 diabetic subjects will therefore benefit from methods provided herein.

Accordingly, in certain embodiments of all methods provided herein, the subject has type 1 diabetes.

Although characterized by different etiological mechanisms, type 2 diabetes also leads to Langerhans islet degeneration. For example, the insulin resistance characteristic of type 2 diabetes places demands on the subject's beta cells to produce more insulin, ultimately leading to exhaustion and degeneration of the pancreatic islet cells. Therefore, regeneration of pancreatic islet cells, especially beta cells, is also an unmet medical need for type 2 diabetes mellitus patients. As demonstrated herein, HGF-MET agonists (e.g. MET agonist antibodies) are able to promote islet cell growth in a model of type 2 diabetes, leading to increased numbers of beta cells, increased insulin production and therefore better glycaemic control.

Accordingly, in certain embodiments of all methods provided herein, the subject has type 2 diabetes.

It is further demonstrated herein that administration of HGF-MET agonist, in particular MET agonist antibodies, is able to promote wound healing in a model of diabetes, which exhibit impaired wound healing. Therefore, in one aspect the invention provides a method of promoting wound healing in a diabetic subject, in particular a human patient. In certain embodiments, the diabetic subject has type 1 or type 2 diabetes. The method provides for treatment of diabetes-associated ulcers and wounds by accelerating healing, improving re-epithelization and promoting vascularization of high blood glucose-induced sores.

In Vitro Methods

It is demonstrated herein that growth of pancreatic islet cells is promoted by HGF-MET agonists. As well as being an important effect in vivo, HGF-MET agonists (such as MET agonist antibodies) will be advantageously used for in vitro expansion of pancreatic islet cells. Promoting growth of islet cells in vitro is important, for example, in preparation for islet cell grafts. Pancreatic islets that have been isolated in preparation for grafting will have limited viability in vitro. Contacting the isolated islet cells with an HGF-MET agonist (e.g. an anti-MET agonist antibody) will prolong the survival of the isolated islet cells in vitro. As a result, the window for effective grafting will be prolonged, and a greater proportion of the grafted islets will be viable. Similarly, isolated islets that are to be grafted can be expanded using HGF-MET agonists according to the provided methods, and thereby increase the cell population available for grafting.

Accordingly, in a further aspect is provided an in vitro method for promoting growth of a cell population or tissue comprising pancreatic islet cells, the method comprising contacting the cell population or tissue with an HGF-MET agonist. In preferred embodiments, the HGF-MET agonist is a MET agonist antibody.

The invention also relates to an ex vivo method of preserving an islet cell or pancreas transplant which comprises contacting the islet cell or pancreas transplant with an HGF-MET agonist, preferably a MET agonist antibody.

Subject or Patient

As demonstrated herein, administration of MET agonists (for example a MET agonist antibody) promotes growth of functional pancreatic islet cells. Promoting growth of pancreatic islet cells is especially important for patients either recently diagnosed with diabetes, especially type 1 diabetes, or even in so called “pre-diabetes”.

Typically, type 1 diabetes symptoms become manifest at adolescence. However, when the pathology is diagnosed, the majority of the patient's pancreatic beta cells have been destroyed (greater than 50%, for example 70% or 80% destruction). Langerhans islet cell degeneration occurs rapidly, particularly at the time when clinical symptoms become apparent and a diagnosis of diabetes is most-commonly made—as a result, the time-window for effective therapeutic intervention is narrow. This is evidenced by the fact that treatment with immunosuppressive agents (to restrict pancreatic islet cell degeneration) is most effective soon after diagnosis, preferably within 6 weeks.

Accordingly, in certain embodiments of the methods provided herein, the subject has been diagnosed with diabetes and first administration of the MET agonist (e.g. MET agonist antibody) is within 6 weeks of diagnosis. Preferably the first administration is within 5 weeks, within 4 weeks or within 3 weeks of diagnosis.

In certain embodiments, the subject has “pre-diabetes”. In such embodiments, “pre-diabetes” can be defined according to the American Diabetes Association (ADA) thresholds for fasting plasma glucose (FPG), for oral glucose tolerance test (OGTT), or both FPG and OGTT thresholds.

According to the ADA definition, “pre-diabetes” can be characterised by impaired fasting glucose—that is, a FPG of at least 100 mg/dl (5.6 mmol/l), but less than 126 mg/dl (7.0 mmol/l). Pre-diabetes can also be characterised by impaired glucose tolerance—that is OGTT results of at least 140 mg/dl (7.8 mmol/l) but less than 200 mg/dl (11.1 mmol/l). Patients with a fasting glucose of 126 mg/dl (7.0 mmol/l) or greater have impaired fasting glucose to the extent that they are diagnosed with diabetes. Patients with an OGTT of 200 mg/dl (11.1 mmol/l) or greater have impaired glucose tolerance to the extent that they are diagnosed with diabetes

Promoting islet cell growth in subjects still exhibiting partial glucose control (for example subjects in early stages of diabetes, or “pre-diabetes”) is particularly advantageous because these subjects still have a population of functioning islet cells. Methods according to the invention can therefore prolong the period in which such patients have functioning pancreatic islet cells.

Accordingly, in certain embodiments, the methods provided herein are methods of treating pre-diabetes.

In certain embodiments of the methods provided herein, the subject exhibits a fasting glucose of greater than 5.6 mmol/l. In certain embodiments, the subject exhibits a fasting glucose of greater than 6.1 mmol/l. In certain embodiments, the subject exhibits a fasting glucose of greater than 5.6 mmol/l and less than 7.0 mmol/l. In certain embodiments, the subject exhibits a fasting glucose of greater than 6.1 mmol/l and less than 7.0 mmol/l. In certain embodiments, the subject exhibits a fasting glucose of 7.0 mmol/or greater.

In certain embodiments of the methods provided herein, the subject exhibits a fasting glucose of greater than 100 mg/dl. In certain embodiments, the subject exhibits a fasting glucose of greater than 110 mg/dl. In certain embodiments, the subject exhibits a fasting glucose of greater than 100 mg/dl and less than 126 mg/dl. In certain embodiments, the subject exhibits a fasting glucose of greater than 110 mg/dl and less than 126 mg/dl. In certain embodiments, the subject exhibits a fasting glucose of 126 mg/dl or greater.

In certain embodiments of the methods provided herein, the subject exhibits an OGTT of greater than 7.8. mmol/l. In certain embodiments, the subject exhibits a fasting glucose of greater than 7.8 mmol/l and less than 11.1 mmol/l. In certain embodiments, the subject exhibits a fasting glucose of 11.1 mmol/I or greater.

In certain embodiments of the methods provided herein, the subject exhibits an OGTT of greater than 140 mg/dl. In certain embodiments, the subject exhibits a fasting glucose of greater than 140 mg/dl and less than 200 mg/dl. In certain embodiments, the subject exhibits a fasting glucose of 200 mg/dl or greater.

In certain embodiments of the methods provided herein, the subject is an adolescent—that is, the subject is 10-19 years of age, for example 12-18 years of age.

As already described, the methods provided herein are particularly advantageous for subjects that have depleted islet cell levels but still have a population of functioning islet cells. This is because the methods can promote the survival of the remaining islet cells and at the same time promote growth and regeneration of new islet cells.

Accordingly, in certain embodiments of all methods provided herein, the subject is characterised by having a population of pancreatic islet cells at least 50% smaller than a healthy individual. In certain embodiments, the subject has a population of pancreatic islet cells at least 70%, optionally at least 80%, at least 90%, or at least 95% smaller than a healthy individual. In certain embodiments, the subject has a population of pancreatic islet cells about 70% to about 80% smaller than a healthy individual.

Destruction of pancreatic islet cells by autoantibodies may occur for some time before clinical symptoms become evident and diabetes is diagnosed. During this period, auto-antibodies to islet cell antigens can be detected, indicating ongoing destruction of pancreatic islet cells. The methods provided herein are will be particularly advantageous in subjects in which such antibodies can be detected, especially if the subject is not yet symptomatic, because these subjects will still have a population of functioning islet cells that can be protected and regenerated using the methods.

Accordingly, in certain embodiments, the subject has autoantibodies to islet cell antigens detectable in their serum. In preferred such embodiments, the subject has not been diagnosed with diabetes. In certain embodiments, the method comprises the step of measuring the level of autoantibodies to islet cell antigens in the subject's serum and administering the MET agonist (e.g. MET agonist antibody) if the level is raised compared to the level characteristic of a healthy subject.

Subjects with latent autoimmune diabetes of adults (LADA) will particularly benefit from the methods provided herein. LADA is a form of diabetes in which progression is typically slower than diabetes diagnosed in juveniles. LADA can be characterised by impaired glycaemic control (e.g. hyperglycaemia) together with detection of C-peptide. Subjects may also have detectable antibodies against pancreatic islet cells. Degeneration of pancreatic islet cells (in particular beta islet cells) in LADA patients is slower. As a result, it is expected that these patients will retain a population of functioning islet cells for longer. The methods provided herein can promote the survival of the remaining islet cells and at the same time promote growth and regeneration of new islet cells, and will therefore particularly benefit LADA patients.

Accordingly, in certain embodiments, the subject has LADA. In certain embodiments, the method is a method of treating LADA.

The methods described herein will also be particularly advantageous in subjects that receive a pancreatic tissue transplant. Pancreatic tissue transplant is a possible treatment in subjects (such as diabetic subjects) where the islet cells have been destroyed. Such transplants can be in the form of a whole pancreas transplant, partial transplant of portion of a pancreas, or graft of isolated islets. In all instances, methods provided herein will be particularly advantageous in patients receiving such transplants and grafts, since the methods will promote survival of the transplanted islets and also growth and expansion of those cells.

Accordingly, in certain embodiments of all methods provided herein, the subject has previously received a pancreatic tissue transplant. In certain embodiments, the subject has previously received a whole pancreas transplant. In certain embodiments, the subject has previously received a partial pancreas transplant. In certain embodiments, the subject has previously received a pancreatic islet graft.

In preferred embodiments of all methods provided herein, the subject has type 1 diabetes. In preferred embodiments of all methods provided herein, the subject has type 2 diabetes.

As described elsewhere herein, the provided methods are particularly advantageous in a pancreatic tissue transplant context. In this context, the methods are particularly advantageous in promoting growth of the transplanted pancreatic islet cells. However, the methods are also advantageous when administered to a healthy subject from which pancreatic islet cells may be taken—i.e. a donor subject. As demonstrated herein, administration of an HGF-agonist (in particular a MET agonist antibody) to a healthy subject promotes growth of their pancreatic islet cells without adverse effects. Therefore, a healthy subject from which pancreatic tissue is going to be taken for transplant—i.e. a donor subject—will benefit from administration of an HGF-MET agonist (e.g. a MET agonist antibody) according to the provided methods, as doing so will promote growth of their pancreatic islet cells, thereby providing more cells for transplant. In addition, if the donor is a live donor, the remaining islet cell population will be larger following HGF-MET agonist administration.

Accordingly, in certain embodiments of the provided methods, the subject is a healthy donor subject.

In preferred embodiments of all aspects, the subject or patient is a mammal, preferably a human.

In preferred embodiments of all aspects, the subject is a subject in need of the method—i.e. the method is administered to a subject in need thereof.

Combination Therapies

HGF-MET agonists administered according to the methods provided herein are particularly advantageous when administered as a combination therapy with immunosuppressive therapies. This is because immunosuppressive agents can reduce the autoimmune-mediated islet cell destruction. However, repeated doses of immunosuppressive agents over a period of weeks and months can be required in order for this protection to take effect. During this lag period, the islet cells can continue to degenerate, often to the point that they are completely destroyed by the time the immunosuppressive takes clinical effect. Administration of an HGF-MET agonist according to the present invention can prolong the survival of islet cells. The treatment window for immunosuppressives to become effective is therefore lengthened, meaning the combination treatment is more likely to be effective at protecting the subject's islet cells. Moreover, as well prolonging survival of islet cells, the methods provided herein promote their growth. The combination therapy will therefore be more effective as a result of a longer effective treatment window for the immunosuppressive agent to reduce islet cell degradation alongside growth and expansion of new islet cells as a result of the MET agonist administration.

Accordingly, in certain embodiments of all methods and second medical indication uses provided herein, the subject is further administered one or more immunosuppressive agent. Accordingly, in certain embodiments, it is also provided an HGF-MET agonist for use in combination with one or more immunosuppressive agent for promoting pancreatic islet cell growth, for promoting insulin production, and/or for treating diabetes in a subject. Also provided is an HGF-MET agonist for use for promoting pancreatic islet cell growth, for promoting insulin production, and/or for treating diabetes in a subject who/which is undergoing therapy with one or more immunosuppressive agent.

The immunosuppressive agent will reduce autoimmune mediated degradation of islet cells. In certain embodiments, the one or more immunosuppressive agent is selected from the list consisting of: cyclosporin A; mycophenolate, vitamin D3, an anti-CD3 antibody, an anti-IL-21 antibody, an anti-CD20 antibody (e.g. rituximab), an anti-CTLA4 antibody, an anti-TNFα antibody (e.g. infliximab), an anti-IL1α antibody, an anti-IL1 β antibody, anti-CD4 antibody, an anti-CD45 antibody, a CTLA4 molecule (e.g. abatacept), a TNFα inhibitor (e.g. etanercept), a PD-L1 molecule, an IL-1 receptor antagonist (e.g anakinra), pegylated granulocyte colony-stimulating factor (e.g. pegfilgrastim), human recombinant IFN-alpha, IL-10, Glutamic Acid Decarboxylase (GAD)-65, tolerising insulin peptides (e.g. insulin B:9-23, Proinsulin peptide 19-A3), DiaPep277 of HSP60, regulatory T cells (Tregs), and tolerising dendritic cells. For example, GAD-65 and IL-10 may be administered together, for instance as a transgenic bacteria (e.g. Lactococcus) expressing both molecules.

Combinations of administration of MET agonists (e.g. MET agonist antibodies) with immunosuppressive agents is particularly advantageous for subjects exhibiting early stage diabetes, or subjects exhibiting impaired glucose control. Particularly preferred patients or subjects are those described in the “Subject or Patient” section herein.

For example, it may be particularly advantageous in subjects exhibiting a fasting glucose level of greater than 5.6 mmol/l, for example greater than 5.6 mmol/l and less than 7.0 mmol/l. Although these patients have a proportion of their islet cells depleted, they still have a population of islet cells. By combining immunosuppressives and a MET agonist according to the methods provided herein, the remaining islet cell population can be protected from degradation and the growth of new islet cells promoted.

In certain embodiments, the methods and second medical indication uses provided herein are used in combination with an anti-diabetes medication. Examples of diabetes therapies include insulin, diet management, metformin, sulfonylureas, thiazoidinediones, dipeptidyl peptidase-4 inhibitors, SGLT2 inhibitors, and glucagon-like peptide-1 analogs. Accordingly, in certain embodiments, it is also provided an HGF-MET agonist for use in combination with an anti-diabetes medication for promoting pancreatic islet cell growth, for promoting insulin production, and/or for treating diabetes in a subject. Also provided is an HGF-MET agonist for use for promoting pancreatic islet cell growth, for promoting insulin production, and/or for treating diabetes in a subject who/which is undergoing therapy with an anti-diabetes medication.

Methods and second medical indication uses provided herein may further be advantageously combined with administration of insulin. Insulin therapy can manage the symptoms of a degraded islet cell population during the period in which the methods provided herein is expanding the islet cell population.

Accordingly, in certain embodiments of all aspects of the methods and second medical indication uses provided herein, the subject is administered insulin at least daily—that is, at least once per day, optionally more frequently.

Administration

It will be appreciated that, as used herein, administration of an HGF-MET agonist (for example an anti-MET agonist antibody) to a subject refers to administration of an effective amount of the agonist.

In certain embodiments, the HGF-MET agonist (for example anti-MET agonist antibody or antigen binding fragment thereof) is administered at a dose in the range of from about 0.1 mg/kg to about 40 mg/kg per dose. In certain embodiments, the HGF-MET agonist (for example anti-MET agonist antibody or antigen binding fragment thereof) is administered at a dose in the range of from 0.5 mg/kg to about 35 mg/kg, optionally from about 1 mg/kg to about 30 mg/kg. In certain preferred embodiments, the HGF-MET agonist (for example anti-MET agonist antibody or antigen binding fragment thereof) is administered at a dose in the range of from about 1 mg/kg to about 10 mg/kg. That is, a dose of about 1, 2, 3, 4, 5, 6, 7, 8, 9 or 10 mg/kg. In certain preferred embodiments, the HGF-MET agonist (for example anti-MET agonist antibody or antigen binding fragment thereof) is administered at a dose of 1 mg/kg, 3 mg/kg, 10 mg/kg or 30 mg/kg.

Suitable routes for administration of the HGF-MET agonist (for example an anti-MET agonist antibody) to a subject would be familiar to the skilled person. Preferably the MET agonist is administered parenterally. In certain preferred embodiments, the HGF-MET agonist is administered orally or per os (p.o.), subcutaneously (s.c.), intravenously (i.v.), intradermally (i.d.), intramuscularly (i.m.) or intraperitoneally (i.p.). In certain preferred embodiments, the HGF-MET agonist is a MET agonist antibody and is administered intravenously.

The HGF-MET agonist (for example anti-MET agonist antibody) can be administered according to a regimen that maintains an effective level of the agonist in the subject. The skilled person is familiar with suitable dosage regimens. For example, in certain embodiments, the HGF-MET agonist (e.g. MET agonist antibody) is administered according to a dosage regimen of at least once perweek—that is, a dose is administered approximately every 7 days or more frequently. In certain embodiments, the HGF-MET agonist (e.g. MET agonist antibody) is administered 1-3 times a week (i.e. 1, 2 or 3 times a week). In certain preferred embodiments, the HGF-MET agonist (e.g. MET agonist antibody) is administered twice per week. In certain preferred embodiments, the HGF-MET agonist is a MET agonist antibody and is administered once per week or twice per week.

For the methods described herein, the HGF-MET agonist (e.g. MET agonist antibody) is administered for a period sufficient to achieve effective treatment. The skilled person is able to determine the necessary treatment period for any individual patient. In certain embodiments, the HGF-MET agonist (e.g. a MET agonist antibody) is administered for a treatment period of at least 1 week. In certain embodiments, the HGF-MET agonist (e.g. a MET agonist antibody) is administered for a treatment period of at least 2 weeks, at least 3 weeks, or at least 4 weeks. In certain embodiments, the HGF-MET agonist (e.g. a MET agonist antibody) is administered for a treatment period of at least 1 month, at least 2 months or at least 3 months. In certain preferred embodiments, the HGF-MET agonist is a MET agonist antibody and is administered for a treatment period of 3 months.

It will be appreciated that the HGF-MET agonist (e.g. a MET agonist antibody) may be administered according to any combination of the described doses, dosage regimens and treatment periods. For example, in certain embodiments, the HGF-MET agonist (e.g. a MET agonist antibody) may be administered according to a dosage regimen of twice per week, at a dose of from 1 mg/kg to 5 mg/kg, for a period of at least 3 months. Other embodiments of the methods explicitly include other combinations of the recited doses, dosage regimens and treatment periods.

HGF-MET Agonist

In all aspects of the invention, an HGF-MET agonist is to be administered to a subject or patient. “HGF-MET agonist” and “MET agonist” are used interchangeably to refer to non-native agents that promote signalling via the MET protein—i.e. agents other than HGF that bind MET and increase MET signalling. Such agents may be small molecules, binding proteins such as antibodies or antigen binding fragments, aptamers or fusion proteins. A particular example of a MET agonist is an anti-MET agonist antibody.

Agonist activity on binding of MET by the MET agonists described herein is indicated by molecular and/or cellular responses that (at least partially) mimic the molecular and cellular responses induced upon HGF-MET binding.

Methods for determining MET agonism according to the invention, for example by MET agonist antibodies and antigen binding fragments, would be familiar to the skilled person. For example, MET agonism may be indicated by molecular responses such as phosphorylation of the MET receptor and/or cellular responses, for example those detectable in a cell scattering assay, an anti-apoptosis assay and/or a branching morphogenesis assay.

MET agonism may be determined by the level of phosphorylation of the MET receptor upon binding. In this context, a MET agonist antibody or antigen binding fragment, for example, causes auto-phosphorylation of MET in the absence of receptor-ligand binding—that is, binding of the antibody or antigen binding fragment to MET results in phosphorylation of MET in the absence of HGF. Phosphorylation of MET may be determined by assays known in the art, for example Western Blotting or phospho-MET ELISA (as described in Basilico et al., J Clin Invest. 124, 3172-3186, 2014, incorporated herein by reference).

MET agonism may alternatively be measured by induction of HGF-like cellular responses. MET agonism can be measured using assays such as a cell scattering assay, an anti-apoptosis assay and/or a branching morphogenesis assay. In this context, a MET agonist, for example an antibody or antigen binding fragment, induces a response in cellular assays such as these that resembles (at least partially) the response observed following exposure to HGF.

For example, a MET agonist (for example a MET agonist antibody) may increase cell scattering in response to the antibody compared to cells exposed to a control antibody (e.g. IgG1).

By way of further example, a MET agonist (for example a MET agonist antibody) may exhibit a protective potency against drug-induced apoptosis with an EC50 of less than 32 nM. By way of further example, a MET agonist (for example a MET agonist antibody) may exhibit an Emax cellular viability of greater than 20% compared to untreated cells.

By way of further example, a MET agonist (for example a MET agonist antibody) may increase the number of branches per spheroid in cell spheroid preparations exposed to the antibody or antigen binding fragment.

It is preferred that the MET agonists used according to the invention promote MET signalling to a magnitude of at least 70% of the natural ligand, HGF—that is, that the agonists are “full agonists”. In certain embodiments, the MET agonists promote signalling to a magnitude of at least 80%, optionally at least 85%, at least 90%, at least 95% or at least 96%, at least 97%, at least 98%, at least 99% or at least 100% of HGF.

A MET agonist phosphorylates MET when binding of the MET agonist causes auto-phosphorylation of MET in the absence of receptor-ligand binding—that is, binding of the MET agonist to human hMET results in phosphorylation of hMET in the absence of hHGF and binding of the MET agonist to mMET results in phosphorylation of mMET in the absence of mHGF. Phosphorylation of MET may be determined by assays known in the art, for example Western Blotting or phospho-MET ELISA (as described in Example 6 and in Basilico et al., J Clin Invest. 124, 3172-3186, 2014). In certain embodiments, if MET agonism is determined using a phosphorylation assay, the MET agonist, e.g. a MET antibody, exhibits a potency for MET with an EC50 of <1 nM. In certain embodiments, the MET agonist, e.g. a MET antibody, exhibits a potency for MET agonism of an EMAX of at least 80% (as a percentage of maximal HGF-induced activation).

In certain embodiments, if MET agonism is measured in a cell scattering assay, the MET agonist, for example a MET antibody or antigen binding fragment, induces an increase in cell scattering at least equivalent to 0.1 nM homologous HGF when the antibody concentration is 0.1-1 nM.

In certain embodiments, if MET agonism is measured in an anti-apoptosis assay, the MET agonist (for example a MET antibody or fragment thereof) exhibits an EC50 no more than 1.1× that of HGF. In certain embodiments, if MET agonism is measured in an anti-apoptosis assay, the MET agonist (for example a MET antibody or fragment thereof) exhibits an Emax cellular viability of greater than 90% that observed for HGF.

In certain embodiments, if MET agonism is measured in a branching morphogenesis assay, cells treated with the MET agonist (e.g. a MET antibody or antigen binding fragment) exhibit greater than 90% of the number of branches per spheroid induced by the same (non-zero) concentration of HGF.

HGF-MET agonists particularly preferred in all aspects of the invention are anti-MET agonist antibodies, also referred to herein as “MET agonist antibodies”, “agonist antibodies” and grammatical variations thereof. In other words, MET agonist antibodies (or antigen binding fragments thereof) for use according to the invention bind MET and promote cellular signalling via MET.

Agonistic anti-MET antibodies generated to date are frequently obtained as by-products from processes intending to identify antagonistic molecules and are not designed explicitly to become agonistic molecules for therapeutic use. Moreover, the most manifest limit of the prior art anti-MET antibodies is that they have been generated in a mouse system (except for B7 that was identified using a human naïve phage library); as a result, it is unlikely that these antibodies will display cross-reactivity with mouse MET. Even if a minor cross-reactivity with self-antigens is in principle possible, these interactions have normally a very low affinity. While the absence of cross-reactivity is not a concern for mouse models of cancer (as they employ human xenografts), cross-reactivity of antibodies between human and mouse MET is an important requirement for pre-clinical mouse models of regenerative medicine or non-oncological human diseases, which require the antibody to function on mouse tissues and cells. Not only is it necessary for an agonistic anti-MET antibody to cross-react with mouse MET in order for the antibody to be evaluated in pre-clinical models, but it is desirable that the antibody binds to mouse MET with an affinity the same or similar to its affinity for human MET, and also that the antibody elicits effects in mouse systems the same or similar to the effects which it evokes in human systems—otherwise the experiments conducted in pre-clinical models will not be predictive of the human situation. As demonstrated in the Examples, none of the prior art anti-MET agonistic antibodies exhibit affinity for mouse MET, and certainly none of the prior art antibodies exhibit the same or similar binding and agonistic effects in both mouse and human systems.

The anti-MET antibodies provided herein are characterised by high affinity binding to human MET (hMET), and also high affinity binding with mouse MET (mMET). Binding affinity for hMET and mMET may be assessed using standard techniques known to persons of skill in the art.

In one embodiment, binding affinity of a Fab clone comprising a defined VH/VL pairing may be assessed using surface plasmon resonance, e.g. using the Biacore™ system. Fab clones comprising VH/VL pairings of the antibodies and antigen binding fragments of the invention typically exhibit an off-rate for hMET measured by Biacore™ in the range of from 1×10⁻³ to 1×10⁻² s⁻¹, optionally 1×10⁻³ to 6×10⁻³s⁻¹. An off-rate within this range may be taken as an indication that the Fab, and a corresponding bivalent mAb, exhibit high affinity binding to hMET. Similarly, the Fab clones comprising VH/VL pairings of the antibodies, and antigen binding fragments of the invention typically exhibit an off-rate for mMET measured by Biacore™, as described in the accompanying examples, in the range of from 1×10³ to 1×10⁻² s⁻¹, optionally 1×10⁻³ to 6×10⁻³ s⁻¹. An off-rate within this range may be taken as an indication that the Fab, and a corresponding bivalent mAb, exhibit high affinity binding to mMET. Therefore, Fabs that exhibit off-rates for both human and murine MET falling within the stated ranges show high affinity binding for hMET, and high affinity binding for mMET—that is, the Fabs are cross-reactive between hMET and mMET. Bivalent mAbs comprising two Fabs that (individually) exhibit off-rates for human and murine MET within the stated ranges are also taken to exhibit high affinity binding to human MET and high affinity binding to murine MET.

Binding affinity may also be expressed as the dissociation constant for a particular antibody, or the K_{D}. The lesser the K_{D} value, the stronger the binding interaction between an antibody and its target antigen. K_{D} may be determined, for example, by combining the Kₒₙ and K_{off} rate determined by SPR measurement. Typically, antibodies and antigen binding fragments of the invention, when measured as mAbs, exhibit a K_{D} for mMET and for hMET of less than 0.1 nMol/L.

Binding affinity to human and murine MET can also be assessed using a cell-based system as described in the accompanying examples, in which mAbs are tested for binding to mammalian cell lines that express MET, for example using ELISA or flow cytometry. High affinity for hMET or mMET may be indicated, for example, by an EC₅₀ of no more than 0.5 nM in an ELISA such as that described in Example 3.

The high affinity hMET and mMET cross-reactive antibodies and antigen binding fragments described herein are MET agonists. As used herein, MET agonists induce (partially or fully) MET signalling when binding to the MET receptor. MET agonist antibodies and antigen binding fragments according to the invention are agonists of hMET and mMET. Agonist activity on binding of hMET or mMET by the antibodies described herein may be indicated by molecular and/or cellular responses that (at least partially) mimic the molecular and cellular responses induced upon homologous HGF-MET binding (i.e. human HGF binding hMET, mouse HGF binding mMET). Antibodies stimulating such a response are also referred to herein as “anti-MET agonists”, “agonist antibodies” and grammatical variations thereof. Similarly, antibodies partially or fully stimulating such responses are referred to herein as “partial MET agonists” or“partial agonists”, or “full MET agonists” or “full agonists”, respectively. It is emphasised that antibodies and antigen binding fragments of the invention induce MET signalling in both human and mouse systems—that is, they are agonists of hMET and mMET. Thus the following discussion applies both to the response induced by binding of hMET by the antibodies and antigen binding fragments of the invention, and to the response induced by binding of mMET by the antibodies and antigen binding fragments of the invention.

As demonstrated in the Examples, MET agonist antibodies 71D6 and 71G2 effectively promote pancreatic islet cell growth, especially islet beta cells. 71D6 and 71G2 bind an epitope on the SEMA domain of MET, in particular an epitope on blade 4-5 of the SEMA β-propeller. MET agonist antibodies binding an epitope on the SEMA domain of MET, in particular blade 4-5 of the SEMA β-propeller have therefore been demonstrated to promote pancreatic islet cell growth, especially beta cell growth.

Thus, in certain embodiments, the methods described herein comprise administering a MET agonist antibody or antigen binding fragment thereof, wherein the antibody or antigen binding fragment binds an epitope in the SEMA domain of MET. In certain preferred embodiments, the antibodies or fragments thereof binds an epitope located on a blade of the SEMA β-propeller. In certain embodiments, the epitope is located on blade 4 or 5 of SEMA β-propeller. In certain preferred embodiments, the antibody or antigen binding fragment binds an epitope located between amino acids 314-372 of MET (in particular human MET).

As shown in the Examples, MET agonist antibodies binding the SEMA domain of MET, including 71D6, have been shown to bind to an epitope on MET that includes residue Ile367 and residue Asp371. Mutation at either of these residues impairs binding of the antibodies to MET, with mutation of both residues completely abrogating binding.

Therefore, in certain preferred embodiments the methods described herein comprise administering a MET agonist antibody or antigen binding fragment thereof, wherein the antibody or antigen binding fragment recognises an epitope comprising the amino acid residue Ile367. In certain preferred embodiments the methods described herein comprise administering a MET agonist antibody or antigen binding fragment thereof, wherein the antibody or antigen binding fragment recognises an epitope comprising the amino acid residue Asp371.

In certain preferred embodiments, the antibody or antigen binding fragment binds an epitope comprising the amino acid residues Ile367 and Asp372 of MET.

As well as MET agonist antibodies binding the SEMA domain, also described herein are agonist antibodies binding other MET domains. For example, 71G3 binds an epitope on the PSI domain of MET. As demonstrated in the Examples, antibody 71G3 is also able to promote pancreas islet cell growth in all models tested.

Thus, in certain embodiments the methods described herein comprise administering a MET agonist antibody or antigen binding fragment thereof, wherein the antibody or antigen binding fragment binds an epitope in the PSI domain of MET. In certain preferred embodiments, the antibody or antigen binding fragment binds an epitope located between amino acids 546 and 562 of MET, preferably human MET.

As shown in the Examples, MET agonist antibodies binding the PSI domain of MET, including 71G3, have been shown to bind to an epitope on MET that includes residue Thr555. Mutation at this residue completely abrogated binding of the PSI-binding agonist antibodies to MET.

Therefore, in certain preferred embodiments the methods described herein comprise administering a MET agonist antibody or antigen binding fragment thereof, wherein the antibody or antigen binding fragment recognises an epitope comprising the amino acid residue Thr555.

In certain aspects, the antibodies described herein recognize epitopes in the extracellular domain of MET that comprise one or more amino acid residues conserved across human and mouse MET. In preferred embodiments antibodies described herein recognize epitopes in the extracellular domain of MET that comprise one or more amino acid residues conserved across human MET, mouse MET, rat MET and simian (e.g. cynomolgus) MET.

As the anti-MET agonist antibodies and antigen binding fragments bind epitopes overlapping or close to the binding domain recognised by HGF, the antibodies and antigen binding fragments are able to (at least partially) compete with HGF for binding of the homologous MET (i.e. compete with human HGF for hMET binding and compete with mouse HGF for mMET binding). That is, the antibodies or antigen binding fragments directly or indirectly prevent HGF from binding the homologous MET in a binding assay, for example an ELISA such as that described in Example 5. Therefore, in certain embodiments, the MET agonist antibodies and antigen binding fragments compete with mouse and human HGF for binding of the homologous MET. An antibody or antigen binding fragment that competes with HGF in this way is also referred to herein as a “HGF competitor”. Assays to determine whether an antibody or antigen binding fragment competes with HGF for MET binding are well known to the skilled person—for example, in a competition ELISA an HGF competitor may exhibit an IC₅₀ of no more than 5 nM and/or an Iₘₐₓ (maximum percentage competition at saturation) of at least 50%. Antibodies and antigen binding fragments of the invention compete with mouse HGF for mMET binding and human HGF for hMET binding.

An MET agonist antibody or antigen binding fragment may “fully compete” or “partially compete” with HGF for homologous MET binding. In this context, a “full competitor” may be an antibody or antigen binding fragment that in a competition assay, for example an ELISA, exhibits an IC₅₀ of less than 2 nM and/or an Iₘₐₓ of at least 90%. In certain embodiments, a “full competitor” exhibits an IC₅₀ of less than 1 nM and/or an Iₘₐₓ of greater than 90%. A “partial competitor” may be an antibody or antigen binding fragment that in a competition assay, for example an ELISA, exhibits an IC₅₀ of 2-5 nM and/or an Iₘₐₓ of 50-90%. The given values apply to competition with mouse HGF and human HGF for binding of the homologous MET.

The MET agonist antibodies and antigen binding fragments thereof are advantageous due to their ability to recognise both human and mouse MET. The antibodies or antigen binding fragments thereof described herein are particularly advantageous when they exhibit equivalent properties when binding to mMET and to hMET. This equivalence allows the antibodies to be analysed in pre-clinical murine models of disease with an expectation that the antibodies will exhibit the same or similar properties in a human context.

Therefore, in certain embodiments, the antibodies and binding fragments exhibit equivalent binding affinity for hMET and mMET. In this context, “equivalent binding affinity” is taken as meaning the affinity of the antibody or antigen binding fragment for hMET is 0.5-1.5 times the affinity of that antibody for mMET. In certain embodiments, antibodies and antigen binding fragments exhibit an affinity for hMET 0.8-1.2 times the affinity of that antibody or antigen binding fragment for mMET.

By way of clarification and example, antibodies or antigen binding fragments having equivalent affinity for mMET and hMET may, when measured as a Fab fragment, exhibit an off-rate for hMET that is 0.5-1.5 times that as the off-rate exhibited for mMET. For example, an antibody having equivalent affinity for mMET and hMET which exhibits an off-rate of 2.6×10⁻³ s⁻¹ for mMET would exhibit an off-rate for hMET of 1.3-3.9×10⁻³ s⁻¹. By way of further example, antibodies or antigen binding fragments having equivalent affinity for mMET and hMET may exhibit an EC₅₀ for hMET (determined for example by ELISA or flow cytometry) of 0.5-1.5 times the EC₅₀ of that antibody or fragment for mMET. For example, an antibody having equivalent affinity for mMET and hMET which exhibits an EC₅₀ for mMET of 0.1 nMol/L would exhibit an EC₅₀ for hMET of 0.05-0.15 nMol/L.

In certain embodiments, the antibodies and antigen binding fragments are equivalent agonists of mMET and of hMET. In this context, “equivalence” is taken as meaning the level of MET agonism induced upon binding of hMET is 0.5-1.5 times that of the level of signalling induced upon binding of mMET. In certain embodiments, antibodies and antigen binding fragments of the invention induce MET signalling upon binding of hMET 0.8-1.2 times that of the level of signalling induced upon binding of mMET.

In certain embodiments, the antibodies or antigen binding fragments are equivalent mMET and hMET agonists when measured by at least one assay of MET agonism described herein. For example, the antibodies or antigen binding fragments of the invention may induce equivalent phosphorylation of MET, exhibit equivalent protective efficacies against drug-induced apoptosis, and/or induce equivalent levels of branching in a branching morphogenesis assay. In certain embodiments, the antibodies or antigen binding fragments exhibit equivalent MET agonism when measured by all of the described assays.

By way of clarification, equivalent phosphorylation of MET by an antibody might be detectable as the ECs for that antibody for hMET being 0.5-1.5× the EC₅₀ for mMET. For example, if the EC₅₀ for mMET is 2.9 nM, that antibody would equivalently induce hMET phosphorylation if the EC₅₀ for hMET is in the range of 1.45-4.35 nM. Similarly, equivalent MET agonism indicated in an anti-apoptosis assay may be detectable as the Eₘₐₓ in human cells being 0.5-1.5× the Eₘₐₓ in mouse cells. For example, if the Eₘₐₓ in mouse cells was 37.5%, that antibody would be an equivalent hMET agonist if the Eₘₐₓ for human cells is in the range of 18.75-56.25%. Equivalent MET agonism indicated in a branching morphogenesis assay may be detectable as the number of branches observed following exposure of human cell spheroids to the antibody being 0.5-1.5× the number of branches observed following exposure of mouse cell spheroids to the same (non-zero) concentration of the antibody. For example, if the number of branches exhibited by mouse cells following exposure to 0.5 nM antibody was 14, that antibody would be an equivalent hMET agonist if the number of branches exhibited by human cells following exposure to 0.5 nM antibody is in the range of 7-21.

Similarly, equivalent agonism of hMET and mMET may be indicated by equivalent cell scattering. The nature of the output of such an assay means application of a 0.5-1.5 factor is not appropriate. In a cell scattering assay, equivalent agonism of hMET and mMET may be indicated by the cell scattering score for human cells exposed to an antibody being +/−1 the cell scattering score for mouse cells exposed to the same antibody at the same (non-zero) concentration. For example, if mouse cells exposed to 0.33 nM of an antibody exhibited a cell scattering score of 2, the antibody would be an equivalent agonist of hHGF if human cells exposed to 0.33 nM of the same antibody exhibited a cell scattering score of 1-3.

In certain embodiments, the antibodies and antigen binding fragments exhibit equivalent HGF competition between mMET and hMET. In this context, “equivalent HGF competition” is taken as meaning the level of competition exhibited by the antibody or antigen binding fragment with human HGF for hMET is 0.5-1.5 times the level of competition exhibited by the antibody or antigen binding fragment with mouse HGF for mMET. In certain embodiments, antibodies and antigen binding fragments of the invention exhibit a level for competition with human HGF 0.8-1.2 times the level of competition exhibited by that antibody or antigen binding fragment with mouse HGF for mMET.

By way of example, equivalent competition by an antibody with human HGF and mouse HGF might be detectable as the IC₅₀ for that antibody competing with human HGF-hMET binding being 0.5-1.5 times the IC₅₀ for that antibody competing with mouse HGF-mMET binding. For example, if the IC₅₀ for mHGF-mMET binding is 0.34 nM, an antibody competes with hHGF and mHGF equivalently if the IC₅₀ for hHGF-hMET binding is in the range of 0.17-0.51 nM.

In certain embodiments, the antibodies and antigen binding fragments are cross-reactive with rat MET and/or macaque MET. Cross-reactivity with one or both of rat and macaque MET has the advantage that toxicology studies can be conducted in rat and/or macaque model systems. In this regard, whether or not an antibody exhibits cross-reactivity with a cynomolgus or rat MET can be determined by ELISA.

Monoclonal antibodies or antigen-binding fragments thereof that “cross-compete” with the MET antibodies disclosed herein are those that bind human MET at site(s) that are identical to, or overlapping with, the site(s) at which the present MET antibodies bind and bind mouse MET at site(s) that are identical to, or overlapping with, the site(s) at which the present MET antibodies bind. Competing monoclonal antibodies or antigen-binding fragments thereof can be identified, for example, via an antibody competition assay. For example, a sample of purified or partially purified human MET can be bound to a solid support. Then, an antibody compound or antigen binding fragment thereof of the present invention and a monoclonal antibody or antigen-binding fragment thereof suspected of being able to compete with such invention antibody compound are added. One of the two molecules is labelled. If the labelled compound and the unlabelled compound bind to separate and discrete sites on MET, the labelled compound will bind to the same level whether or not the suspected competing compound is present. However, if the sites of interaction are identical or overlapping, the unlabelled compound will compete, and the amount of labelled compound bound to the antigen will be lowered. If the unlabelled compound is present in excess, very little, if any, labelled compound will bind. For purposes of the present invention, competing monoclonal antibodies or antigen-binding fragments thereof are those that decrease the binding of the present antibody compounds to MET by about 50%, about 60%, about 70%, about 80%, about 85%, about 90%, about 95%, or about 99%. Details of procedures for carrying out such competition assays are well known in the art and can be found, for example, in Harlow and Lane, Antibodies, A Laboratory Manual, Cold Spring Harbor Laboratory Press, Cold Spring Harbor, N.Y., 1988, 567-569, 1988, ISBN 0-87969-314-2. Such assays can be made quantitative by using purified antibodies. A standard curve is established by titrating one antibody against itself, i.e., the same antibody is used for both the label and the competitor. The capacity of an unlabelled competing monoclonal antibody or antigen-binding fragment thereof to inhibit the binding of the labelled molecule to the plate is titrated. The results are plotted, and the concentrations necessary to achieve the desired degree of binding inhibition are compared.

Examples of MET agonist antibodies particularly suitable for use in the methods described herein are those having a combination of CDRs corresponding to the CDRs of an anti-MET antibody described herein. Therefore, in certain embodiments, the antibody or antigen binding fragment comprises a combination of VH and VL CDR sequences corresponding to a combination of VH CDRs from a MET agonist antibody described in Table 3 and the corresponding combination of VL CDRs for the same antibody in Table 4.

In certain such embodiments, the antibody or antigen binding fragment comprises a combination of CDRs corresponding to a combination of VH CDRs from a MET agonist antibody described in Table 3 and the corresponding combination of VL CDRs for the same antibody in Table 4, and further having VH and VL domains with at least 90%, optionally at least 95%, optionally at least 99%, preferably 100% sequence identity with the corresponding VH and VL sequences of the antibody described in Table 6. By way of clarification, in such embodiments the permitted variation in percentage identity of the VH and VL domain sequences is not in the CDR regions.

As demonstrated in the Examples, 71D6, 71G2, and 71G3 are MET agonist antibodies that are “full agonists” of MET. That is, on binding of these antibodies to MET, the signalling response is similar to or even exceeds the response to binding of the native HGF ligand. Each of these antibodies is demonstrated herein to effectively promote pancreatic isle cell growth. Therefore in certain preferred embodiments of all aspects and methods described herein, the method comprises administering an HGF-MET agonist that is a full agonist—that is, an agonist that upon binding promotes MET signalling to an extent similar or in excess of MET signalling upon HGF binding. Examples for measuring MET agonism and examples of the effects of full agonists have already been described herein.

Examples of MET full agonists, such as anti-MET antibodies that are full agonists include 71D6, 71G2, and 71G3, as demonstrated in the Examples. Therefore in particularly preferred embodiments of all the methods described herein, the method comprises administering a MET agonist antibody or antigen binding fragment thereof that is a full agonist of MET.

MET agonist antibodies 71D6, 71G2 and 71G3 have each been shown to effectively promote pancreatic islet cell growth. Therefore, in preferred embodiments of all aspects and methods described herein, the antibody or fragment comprises a combination of CDRs having the corresponding CDR sequences of antibody 71D6 (SEQ ID Nos: 30, 32, 34, 107, 109, and 111), of antibody 71G2 (SEQ ID NOs: 44, 46, 48, 121, 123, and 125), or of antibody 71G3 (SEQ ID Nos: 9, 11, 13, 86, 88, and 90).

In preferred embodiments of all aspects, the MET agonist is a MET agonist antibody or antigen binding fragment thereof having HCDR1 of [71D6] SEQ ID NO: 30, HCDR2 of SEQ ID NO: 32, HCDR3 of SEQ ID NO: 34, LCDR1 of SEQ ID NO: 107, LCDR2 of SEQ ID NO: 109, and LCDR3 of SEQ ID NO: 111.

In preferred such embodiments, the antibody or antigen binding fragment comprises: a VH domain comprising SEQ ID NO: 163 or a sequence at least 90% identical thereto, optionally at least 95%, at least 98% or at least 99% identical thereto; and a VL domain comprising SEQ ID NO: 164 or a sequence at least 95% thereto optionally at least 98% or at least 99% identical thereto. By way of clarification, in such embodiments the permitted variation in percentage identity of the VH and VL domain sequences is not in the CDR regions.

MET agonist antibodies for use as described herein can take various different embodiments in which both a VH domain and a VL domain are present. The term “antibody” herein is used in the broadest sense and encompasses, but is not limited to, monoclonal antibodies (including full length monoclonal antibodies), polyclonal antibodies, multispecific antibodies (e.g., bispecific antibodies), so long as they exhibit the appropriate immunological specificity for a human MET protein and for a mouse MET protein. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” comprise a portion of a full length antibody, generally the antigen binding or variable domain thereof. Examples of antibody fragments include Fab, Fab′, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-1136, 2005, the contents of which are incorporated herein by reference).

In preferred embodiments of all aspects provided herein, the MET agonist antibody or antigen-binding fragment thereof is bivalent.

In non-limiting embodiments, the MET antibodies provided herein may comprise CH1 domains and/or CL domains, the amino acid sequence of which is fully or substantially human. Therefore, one or more or any combination of the CH1 domain, hinge region, CH2 domain, CH3 domain and CL domain (and CH4 domain if present) may be fully or substantially human with respect to its amino acid sequence. Such antibodies may be of any human isotype, for example IgG1 or IgG4.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. Such antibodies may be of any human isotype, with human IgG4 and IgG1 being particularly preferred.

MET agonist antibodies may also comprise constant domains of “human” sequence which have been altered, by one or more amino acid additions, deletions or substitutions with respect to the human sequence, excepting those embodiments where the presence of a “fully human” hinge region is expressly required. The presence of a “fully human” hinge region in the MET antibodies of the invention may be beneficial both to minimise immunogenicity and to optimise stability of the antibody.

The MET agonist antibodies may be of any isotype, for example IgA, IgD, IgE, IgG, or IgM. In preferred embodiments, the antibodies are of the IgG type, for example IgG1, IgG2a and b, IgG3 or IgG4. IgG1 and IgG4 are particularly preferred. Within each of these sub-classes it is permitted to make one or more amino acid substitutions, insertions or deletions within the Fc portion, or to make other structural modifications, for example to enhance or reduce Fc-dependent functionalities.

In non-limiting embodiments, it is contemplated that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites). Depending on the intended use of the MET antibody, it may be desirable to modify the antibody of the invention with respect to its binding properties to Fc receptors, for example to modulate effector function.

In certain embodiments, the MET antibodies may comprise an Fc region of a given antibody isotype, for example human IgG1, which is modified in order to reduce or substantially eliminate one or more antibody effector functions naturally associated with that antibody isotype. In non-limiting embodiments, the MET antibody may be substantially devoid of any antibody effector functions. In this context, “antibody effector functions” include one or more or all of antibody-dependent cellular cytotoxicity (ADCC), complement-dependent cytotoxicity (CDC) and antibody-dependent cellular phagocytosis (ADCP).

The amino acid sequence of the Fc portion of the MET antibody may contain one or more mutations, such as amino acid substitutions, deletions or insertions, which have the effect of reducing one or more antibody effector functions (in comparison to a wild type counterpart antibody not having said mutation). Several such mutations are known in the art of antibody engineering. Non-limiting examples, suitable for inclusion in the MET antibodies described herein, include the following mutations in the Fc domain of human IgG4 or human IgG1: N297A, N297Q, LALA (L234A, L235A), AAA (L234A, L235A, G237A) or D265A (amino acid residues numbering according to the EU numbering system in human IgG1).

The antibodies or antigen binding fragments thereof described herein may comprise at least one hypervariable loop or complementarity determining region obtained from a VH domain or a VL domain of a species in the family Camelidae. In particular, the antibody or antigen binding fragment may comprise VH and/or VL domains, or CDRs thereof, obtained by active immunisation of outbred camelids, e.g. llamas, with a human MET antigen.

By “hypervariable loop or complementarity determining region obtained from a VH domain or a VL domain of a species in the family Camelidae” is meant that hypervariable loop (HV) or CDR has an amino acid sequence which is identical, or substantially identical, to the amino acid sequence of a hypervariable loop or CDR which is encoded by a Camelidae immunoglobulin gene. In this context “immunoglobulin gene” includes germline genes, immunoglobulin genes which have undergone rearrangement, and also somatically mutated genes. Thus, the amino acid sequence of the HV or CDR obtained from a VH or VL domain of a Camelidae species may be identical to the amino acid sequence of a HV or CDR present in a mature Camelidae conventional antibody. The term “obtained from” in this context implies a structural relationship, in the sense that the HVs or CDRs of the MET antibody embody an amino acid sequence (or minor variants thereof) which was originally encoded by a Camelidae immunoglobulin gene. However, this does not necessarily imply a particular relationship in terms of the production process used to prepare the MET antibody.

Camelid-derived MET antibodies may be derived from any camelid species, including inter alia, llama, dromedary, alpaca, vicuna, guanaco or camel.

MET antibodies comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. camelid and human.

Camelid-derived CDRs may comprise one of the CDR sequences shown in Tables 3 and 4 below.

In one embodiment the entire VH domain and/or the entire VL domain may be obtained from a species in the family Camelidae. In specific embodiments, the camelid-derived VH domain may comprise the amino acid sequence shown as SEQ ID NOs: 157, 163, or 167, whereas the camelid-derived VL domain may comprise the amino acid sequence shown as SEQ ID NOs: 158, 164, or 168. The camelid-derived VH domain and/or the camelid-derived VL domain may then be subject to protein engineering, in which one or more amino acid substitutions, insertions or deletions are introduced into the camelid amino acid sequence. These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain.

Isolated camelid VH and VL domains obtained by active immunisation of a camelid (e.g. llama) with a human MET antigen can be used as a basis for engineering MET antibodies according to the invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain. The purpose of such changes in primary amino acid sequence may be to reduce presumably unfavourable properties (e.g. immunogenicity in a human host (so-called humanization), sites of potential product heterogeneity and or instability (glycosylation, deamidation, isomerisation, etc.) or to enhance some other favourable property of the molecule (e.g. solubility, stability, bioavailability, etc.). In other embodiments, changes in primary amino acid sequence can be engineered in one or more of the hypervariable loops (or CDRs) of a Camelidae VH and/or VL domain obtained by active immunisation. Such changes may be introduced in order to enhance antigen binding affinity and/or specificity, or to reduce presumably unfavourable properties, e.g. immunogenicity in a human host (so-called humanization), sites of potential product heterogeneity and or instability, glycosylation, deamidation, isomerisation, etc., or to enhance some other favourable property of the molecule, e.g. solubility, stability, bioavailability, etc.

Thus, in one embodiment, a variant MET antibody is used which contains at least one amino acid substitution in at least one framework or CDR region of either the VH domain or the VL domain in comparison to a camelid-derived VH or VL domain, examples of which include but are not limited to the camelid VH domains comprising the amino acid sequences shown as SEQ ID NOs: 157, 163, or 167, and the camelid VL domains comprising the amino acid sequences show as SEQ ID NO: 158, 164, or 168.

In certain embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from llama (prior to introduction of engineered amino acid sequence variation). In such embodiments both the VH and the VL domain may be derived from a single animal, particularly a single animal which has been actively immunised with a human MET antigen.

The invention can, in certain embodiments, encompass chimeric camelid/human antibodies, and in particular chimeric antibodies in which the VH and VL domains are of fully camelid sequence (e.g. Llama or alpaca) and the remainder of the antibody is of fully human sequence. MET antibodies can include antibodies comprising “humanised” or “germlined” variants of camelid-derived VH and VL domains, or CDRs thereof, and camelid/human chimeric antibodies, in which the VH and VL domains contain one or more amino acid substitutions in the framework regions in comparison to camelid VH and VL domains obtained by active immunisation of a camelid with a human MET antigen. Such “humanisation” increases the % sequence identity with human germline VH or VL domains by replacing mis-matched amino acid residues in a starting Camelidae VH or VL domain with the equivalent residue found in a human germline-encoded VH or VL domain.

The invention can, in certain embodiments, encompass chimeric camelid/mouse antibodies, and in particular chimeric antibodies in which the VH and VL domains are of fully camelid sequence (e.g. Llama or alpaca) and the remainder of the antibody is of fully mouse sequence.

MET antibodies and antigen binding fragments of the invention may also be CDR-grafted antibodies in which CDRs (or hypervariable loops) derived from a camelid antibody, for example a camelid MET antibody raised by active immunisation with human MET protein, or otherwise encoded by a camelid gene, are grafted onto a human VH and VL framework, with the remainder of the antibody also being of fully human origin. Such CDR-grafted MET antibodies may contain CDRs having the amino acid sequences shown in Tables 3 and 4 below.

Camelid-derived MET antibodies include variants wherein the hypervariable loop(s) or CDR(s) of the VH domain and/or the VL domain are obtained from a conventional camelid antibody raised against human MET, but wherein at least one of said (camelid-derived) hypervariable loops or CDRs has been engineered to include one or more amino acid substitutions, additions or deletions relative to the camelid-encoded sequence. Such changes include “humanisation” of the hypervariable loops/CDRs. Camelid-derived HVs/CDRs which have been engineered in this manner may still exhibit an amino acid sequence which is “substantially identical” to the amino acid sequence of a camelid-encoded HV/CDR. In this context, “substantial identity” may permit no more than one, or no more than two amino acid sequence mis-matches with the camelid-encoded HV/CDR. Particular embodiments of the MET antibody may contain humanised variants of the CDR sequences shown in Tables 3 and 4.

Camelid (e.g. llama) conventional antibodies provide an advantageous starting point for the preparation of antibodies with utility as human therapeutic agents due to the following factors, discussed in U.S. Ser. No. 12/497,239 which is incorporated herein by reference:

1) High % sequence homology between camelid VH and VL domains and their human counterparts;

2) High degree of structural homology between CDRs of camelid VH and VL domains and their human counterparts (i.e. human-like canonical fold structures and human-like combinations of canonical folds).

The camelid (e.g. llama) platform also provides a significant advantage in terms of the functional diversity of the MET antibodies which can be obtained.

The utility of MET antibodies comprising camelid VH and/or camelid VL domains for human therapy can be improved still further by “humanisation” of natural camelid VH and VL domains, for example to render them less immunogenic in a human host. The overall aim of humanisation is to produce a molecule in which the VH and VL domains exhibit minimal immunogenicity when introduced into a human subject, whilst retaining the specificity and affinity of the antigen binding site formed by the parental VH and VL domains.

One approach to humanisation, so-called “germlining”, involves engineering changes in the amino acid sequence of a camelid VH or VL domain to bring it closer to the germline sequence of a human VH or VL domain.

Determination of homology between a camelid VH (or VL) domain and human VH (or VL) domains is a critical step in the humanisation process, both for selection of camelid amino acid residues to be changed (in a given VH or VL domain) and for selecting the appropriate replacement amino acid residue(s).

An approach to germlining of camelid conventional antibodies has been developed based on alignment of a large number of novel camelid VH (and VL) domain sequences, typically somatically mutated VH (or VL) domains which are known to bind a target antigen, with human germline VH (or VL) sequences, human VH (and VL) consensus sequences, as well as germline sequence information available for llama pacos.

This procedure, described in WO 2011/080350, contents of which are incorporated by reference, can be applied to (i) select “camelid” amino acid residues for replacement in a camelid-derived VH or VL domain or a CDR thereof, and (ii) select replacement “human” amino acid residues to substitute in, when humanising any given camelid VH (or VL) domain. This approach can be used to prepare humanised variants of camelid-derived CDRs having the amino acid sequences shown in Tables 3 and 4 and also for germlining of camelid-derived VH and VL domains having the sequences shown in Table 5.

In certain embodiments of all aspects of the invention, therefore, the anti-MET agonist antibody is an agonist antibody of both human MET and mouse MET.

Pharmaceutical Compositions

Also provided in accordance with the invention are pharmaceutical compositions for use in the methods described herein. Therefore in a further aspect of the invention is provided a pharmaceutical composition comprising an HGF-MET agonist, for example an anti-MET agonist antibody, and a pharmaceutically acceptable excipient or carrier for use in a method according to the invention. Suitable pharmaceutically acceptable carriers and excipients would be familiar to the skilled person. Examples of pharmaceutically acceptable carriers and excipients suitable for inclusion in pharmaceutical compositions of the invention include sodium citrate, glycine, polysorbate (e.g. polysorbate 80) and saline solution.

In certain embodiments, the MET agonist, for example anti-MET agonist antibody, is administered to the subject parenterally, preferably intravenously (i.v.). In certain embodiments the MET agonist, for example anti-MET agonist antibody, is administered as a continuous i.v. infusion until the desired dose is achieved.

In certain embodiments, the MET agonist, for example anti-MET agonist antibody, is administered to the subject parentally, preferably intraperitoneally (i.p.).

### EXAMPLES

The invention will be further understood with reference to the following non-limiting experimental examples.

### Example 1: Generation of Ant-MET Agonist Antibodies—Immunization of Llamas

Immunizations of llamas and harvesting of peripheral blood lymphocytes (PBLs) as well as the subsequent extraction of RNA and amplification of antibody fragments were performed as described (De Haard et al., J. Bact. 187:4531-4541, 2005). Two adult llamas (Lama glama) were immunized by intramuscular injection of a chimeric protein consisting of the extracellular domain (ECD) of human MET fused to the Fc portion of human IgG1 (MET-Fc; R&D Systems). Each llama received one injection per week for six weeks, for a total of six injections. Each injection consisted in 0.2 mg protein in Freund's Incomplete Adjuvant in the neck divided over two spots.

Blood samples of 10 ml were collected pre- and post-immunization to investigate the immune response. Approximately one week after the last immunization, 400 ml of blood was collected and PBLs were obtained using the Ficoll-Paque method. Total RNA was extracted by the phenol-guanidine thiocyanate method (Chomczynski et al., Anal. Biochem. 162:156-159, 1987) and used as template for random cDNA synthesis using the SuperScript™ III First-Strand Synthesis System kit (Life Technologies). Amplification of the cDNAs encoding the VH-CH1 regions of llama IgG1 and VL-CL domains (κ and λ) and subcloning into the phagemid vector pCB3 was performed as described (de Haard et al., J Biol Chem. 274:18218-18230, 1999). The E. coli strain TG1 (Netherland Culture Collection of Bacteria) was transformed using recombinant phagemids to generate 4 different Fab-expressing phage libraries (one A and one K library per immunized llama). Diversity was in the range of 10⁸-10⁹.

The immune response to the antigen was investigated by ELISA. To this end, we obtained the ECDs of human MET (UniProtKB #P08581; aa 1-932) and of mouse MET (UniProtKB #P16056.1; aa 1-931) by standard protein engineering techniques. Human or mouse MET ECD recombinant protein was immobilized in solid phase (100 ng/well in a 96-well plate) and exposed to serial dilutions of sera from llamas before (day 0) or after (day 45) immunization. Binding was revealed using a mouse anti-Ilama IgG1 (Daley et al., Clin. Vaccine Immunol. 12, 2005) and a HRP-conjugated donkey anti-mouse antibody (Jackson Laboratories). Both llamas displayed an immune response against human MET ECD. Consistent with the notion that the extracellular portion of human MET displays 87% homology with its mouse orthologue, a fairly good extent of cross-reactivity was also observed with mouse MET ECD.

### Example 2: Selections and Screenings of Fabs Binding to Both Human and Mouse MET

Fab-expressing phages from the libraries described above were produced according to standard phage display protocols. For selection, phages were first adsorbed to immobilized recombinant human MET ECD, washed, and then eluted using trypsin. After two cycles of selection with human MET ECD, two other cycles were performed in the same fashion using mouse MET ECD. In parallel, we also selected phages alternating a human MET ECD cycle with a mouse MET ECD cycle, for a total of four cycles. Phages selected by the two approaches were pooled together and then used to infect TG1 E. coli. Individual colonies were isolated and secretion of Fabs was induced using IPTG (Fermentas). The Fab-containing periplasmic fraction of bacteria was collected and tested for its ability to bind human and mouse MET ECD by Surface Plasmon Resonance (SPR). Human or mouse MET ECD was immobilized on a CM-5 chip using amine coupling in sodium acetate buffer (GE Healthcare). The Fab-containing periplasmic extracts were loaded into a BIACORE 3000 apparatus (GE Healthcare) with a flow rate of 30 μl/min. The Fab off-rates (k_{off}) were measured over a two minute period. Binding of Fabs to human and mouse MET was further characterized by ELISA using MET ECD in solid phase and periplasmic crude extract in solution. Because Fabs are engineered with a MYC flag, binding was revealed using HRP-conjugated anti-MYC antibodies (ImTec Diagnostics).

Fabs that bound to both human and mouse MET in both SPR and ELISA were selected and their corresponding phages were sequenced (LGC Genomics). Cross-reactive Fab sequences were divided into families based on VH CDR3 sequence length and content. VH families were given an internal number not based on IMTG (International Immunogenetics Information System) nomenclature. Altogether, we could identify 11 different human/mouse cross-reactive Fabs belonging to 8 VH families. The CDR and FR sequences of heavy chain variable regions are shown in Table 3. The CDR and FR sequences of light chain variable regions are shown in Table 4. The full amino acid sequences of heavy chain and light chain variable regions are shown in Table 5. The full DNA sequences of heavy chain and light chain variable regions are shown in Table 6.

TABLE 3
Framework regions and CDR sequences for VH domains of Fabs
binding to both human and mouse MET.

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 76H10 | QLQLVESG | 1 | TYYMT | 2 | WVRQAPG | 3 | DINSGGG | 4 | RFTISRDNAKNT | 5 | VRIWPVG | 6 | WGQGTQ | 7 |
| | GGLVQPGG | | | | KGLEWVS | | TYYADSV | | LYLQMNSLKPED | | YDY | | VTVSS | |
| | SLRVSCTA | | | | | | KG | | TALYYCVR | | | | | |
| | SGFTFN | | | | | | | | | | | | | |
| 71G3 | QVQLVESG | 8 | TYYMS | 9 | WVRQAPG | 10 | DIRTDGG | 11 | RFTMSRDNAKNT | 12 | TRIFPSG | 13 | WGQGTQ | 14 |
| | GGLVQPGG | | | | KGLEWVS | | TYYADSV | | LYLQMNSLKPED | | YDY | | VTVSS | |
| | SLRVSCAA | | | | | | KG | | TALYYCAR | | | | | |
| | SGFTFS | | | | | | | | | | | | | |
| 71C3 | QLQLVESG | 15 | SHAMS | 16 | WVRQAPG | 17 | AINSGGG | 18 | RFTISRDNAKNT | 19 | ELRFDLA | 20 | WGQGTQ | 21 |
| | GGLVQPGG | | | | KGLEWVS | | STSYADS | | LYLQMNSLKPED | | RYTDYEA | | VTVSS | |
| | SLRLSCAA | | | | | | VKG | | TAVYYCAK | | WDY | | | |
| | SGFTFS | | | | | | | | | | | | | |
| 71D4 | ELQLVESG | 22 | GYGMS | 23 | WVRQAPG | 24 | DINSGGG | 25 | RFTISRDNAKNT | 26 | DMRLYLA | 27 | WGQGTQ | 28 |
| | GGLVQPGG | | | | KGLEWVS | | STSYADS | | LYLQMNSLKPED | | RYNDYEA | | VTVSS | |
| | SLRLSCAA | | | | | | VKG | | TAVYYCAK | | WDY | | | |
| | SGFTFS | | | | | | | | | | | | | |
| 71D6 | ELQLVESG | 29 | SYGMS | 30 | WVRQAPG | 31 | AINSYGG | 32 | RFTISRDNAKNT | 33 | EVRADLS | 34 | WGQGTQ | 35 |
| | GGLVQPGG | | | | KGLEWVS | | STSYADS | | LYLQMNSLKPEDV | | RYNDYES | | VTVSS | |
| | SLRLSCAA | | | | | | VKG | | TAVYYCAK | | YDY | | | |
| | SGFTFS | | | | | | | | | | | | | |
| 71A3 | EVQLVESG | 36 | DYDIT | 37 | WVRQAPG | 38 | TITSRSG | 39 | RFTISGDNAKNT | 40 | VYATTWD | 41 | WGKGTL | 42 |
| | GGLVQPGG | | | | KGLEWVS | | STSYVDS | | LYLQMNSLKPED | | VGPLGYG | | VTVSS | |
| | SLRLSCAA | | | | | | VKG | | TAVYYCAK | | MDY | | | |
| | SGFSFK | | | | | | | | | | | | | |
| 71G2 | EVQLQESG | 43 | IYDMS | 44 | WVRQAPG | 45 | TINSDGS | 46 | RFTISRDNAKNT | 47 | VYGSTWD | 48 | WGKGTL | 49 |
| | GGLVQPGG | | | | KGLEWVS | | STSYVDS | | LYLQMNSLKPED | | VGPMGYG | | VTVSS | |
| | SLRLSCAA | | | | | | VKG | | TAVYYCAK | | MDY | | | |
| | SGFTFS | | | | | | | | | | | | | |
| 76G7 | QVQLVESG | 50 | NYYMS | 51 | WVRQAPG | 52 | DIYSDGS | 53 | RFTISRDNAKNT | 54 | VKIYPGG | 55 | WGQGTQ | 56 |
| | GNLVQPGG | | | | KGLEWVS | | TTWYSDS | | LSLQMNSLKSED | | YDA | | VTVSS | |
| | SLRLSCAA | | | | | | VKG | | TAVYYCAR | | | | | |
| | SGFTFS | | | | | | | | | | | | | |
| 71G12 | QVQLQESG | 57 | RYYMS | 58 | WVRQAPG | 59 | SIDSYGY | 60 | RFTISRDNAKNT | 61 | AKTTWSY | 62 | WGQGTQ | 63 |
| | GDLVQPGG | | | | KGLEWVS | | STYYTDS | | LYLQMNSLKPED | | DY | | VTVSS | |
| | SLRVSCVV | | | | | | VKG | | TALYYCAR | | | | | |
| | SGFTFS | | | | | | | | | | | | | |
| 74C8 | EVQLVESG | 64 | NYHMS | 65 | WVRQVPG | 66 | DINSAGG | 67 | RFTISRDNAKNT | 68 | VNVWGVN | 69 | WGKGTL | 70 |
| | GGLVQPGG | | | | KGFEWIS | | STYYADS | | LYLEMNSLKPED | | Y | | VSVSS | |
| | SLRLSCAA | | | | | | VKG | | TALYYCAR | | | | | |
| | SGFTFR | | | | | | | | | | | | | |
| 72F8 | ELQLVESG | 71 | NYVMS | 72 | WVRQAPG | 73 | DTNSGGS | 74 | RFTISRDNAKNT | 75 | SFFYGMN | 76 | WGKGTQ | 77 |
| | GGLVQPGG | | | | KGLEWVS | | TSYADSV | | LYLQMNSLKPED | | Y | | VTVSS | |
| | SLRLSCAA | | | | | | KG | | TALYYCAR | | | | | |
| | SGFTFS | | | | | | | | | | | | | |

TABLE 4
Framework regions and CDR sequences for VL domains of Fabs
binding to both human and mouse MET.

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 76H10 | QAVVTQEP | 78 | GLSSGSV | 79 | WFQQTPGQ | 80 | NTNNRHS | 81 | GVPSRFSGSISG | 82 | SLYTGS | 83 | FGGGTH | 84 |
| | SLSVSPGG | | TTSNYPG | | APRTLIY | | | | NKAALTITGAQP | | YTTV | | LTVL | |
| | TVTLTC | | | | | | | | EDEADYYC | | | | | |
| 71G3 | QAVVTQEP | 85 | GLSSGSV | 86 | WFQQTPGQ | 87 | NTNSRHS | 88 | GVPSRFSGSISG | 89 | SLYPGS | 90 | FGGGTH | 91 |
| | SLSVSPGG | | TTSNYPG | | APRTLIY | | | | NKAALTIMGAQP | | TTV | | LTVL | |
| | TVTLTC | | | | | | | | EDEADYYC | | | | | |
| 71C3 | SYELTQPS | 92 | QGGSLGS | 93 | WYQQKPGQ | 94 | DDDSRPS | 95 | GIPERFSGSSSG | 96 | QSADSS | 97 | FGGGTH | 98 |
| | ALSVTLGQ | | SYAH | | APVLVIY | | | | GTATLTISGAQA | | GNAAV | | LTVL | |
| | TAKITC | | | | | | | | EDEGDYYC | | | | | |
| 71D4 | SSALTQPS | 99 | QGGSLGS | 100 | WYQQKPGQ | 101 | DDDSRPS | 102 | GIPERFSGSSSG | 103 | QSADSS | 104 | FGGGTH | 105 |
| | ALSVTLGQ | | SYAH | | APVLVIY | | | | GTATLTISGAQA | | GNAAV | | LTVL | |
| | TAKITC | | | | | | | | EDEGDYYC | | | | | |
| 71D6 | QPVLNQPS | 106 | QGGSLGA | 107 | WYQQKPGQ | 108 | DDDSRPS | 109 | GIPERFSGSSSG | 110 | QSADSS | 111 | FGGGTH | 112 |
| | ALSVTLGQ | | RYAH | | APVLVIY | | | | GTATLTISGAQA | | GSV | | LTVL | |
| | TAKITC | | | | | | | | EDEGDYYC | | | | | |
| 71A3 | SYELTQPS | 113 | QGGSLGS | 114 | WYQQKPGQ | 115 | DDDSRPS | 116 | GIPERFSGSSSG | 117 | QSADSS | 118 | FGGGTH | 119 |
| | ALSVTLGQ | | SYAH | | APVLVIY | | | | GTATLTISGAQA | | GNAAV | | LTVL | |
| | TAKITC | | | | | | | | EDEGDYYC | | | | | |
| 71G2 | SSALTQPS | 120 | QGGSLGS | 121 | WYQQKPGQ | 122 | GDDSRPS | 123 | GIPERFSGSSSG | 124 | QSTDSS | 125 | FGGGTR | 126 |
| | ALSVSLGQ | | SYAH | | APVLVIY | | | | GTATLTISGAQA | | GNTV | | LTVL | |
| | TARITC | | | | | | | | EDEDDYYC | | | | | |
| 76G7 | QAGLTQPP | 127 | AGNSSDV | 128 | WYQQFPGM | 129 | LVNKRAS | 130 | GITDRFSGSKSG | 131 | ASYTGS | 132 | FGGGTH | 133 |
| | SVSGSPGK | | GYGNYVS | | APKLLIY | | | | NTASLTISGLQS | | NNIV | | LTVL | |
| | TVTISC | | | | | | | | EDEADYYC | | | | | |
| 71G12 | EIVLTQSP | 134 | KSSQSVF | 135 | WYQQRPGQ | 136 | YASTRES | 137 | GIPDRFSGSGST | 138 | QQAYSH | 139 | FGQGTK | 140 |
| | SSVTASVG | | IASNQKT | | SPRLVIS | | | | TDFTLTISSVQP | | PT | | VELK | |
| | GKVTINC | | YLN | | | | | | EDAAVYYC | | | | | |
| 74C8 | QTVVTQEP | 141 | GLSSGSV | 142 | WFQQTPGQ | 143 | NTNSRHS | 144 | GVPSRFSGSISG | 145 | SLYPGS | 146 | FGGGTH | 147 |
| | SLSVSPGG | | TTSNYPG | | APRTLIY | | | | NKAALTITGAQP | | YTNV | | LTVL | |
| | TVTLTC | | | | | | | | EDEADYYC | | | | | |
| 72F8 | QSALTQPP | 148 | TLSSGNN | 149 | WYQQKAGS | 150 | YYTDSRK | 151 | GVPSRFSGSKDA | 152 | SAYKSG | 153 | FGGGTH | 154 |
| | SLSASPGS | | IGSYDIS | | PPRYLLN | | HQDS | | SANAGLLLISGL | | SYRWV | | VTVL | |
| | SVRLTC | | | | | | | | QPEDEADYYC | | | | | |

TABLE 5
Variable domain amino acid sequences of Fabs binding to both human and mouse MET.

| | | SEQ ID | | SEQ ID |
|---|---|---|---|---|
| CLONE | VH | NO. | VL | NO. |
| 76H10 | QLQLVESGGGLVQPGGSLRVSCTASGFTFNTYYMTWVR | 155 | QAVVTQEPSLSVSPGGTVTLTCGLSSGSVTTSNYPGWF | 156 |
| | QAPGKGLEWVSDINSGGGTYYADSVKGRFTISRDNAKN | | QQTPGQAPRTLIYNTNNRHSGVPSRFSGSISGNKAALT | |
| | TLYLQMNSLKPEDTALYYCVRVRIWPVGYDYWGQGTQV | | ITGAQPEDEADYYCSLYTGSYTTVFGGGTHLTVL | |
| | TVSS | | | |
| 71G3 | QVQLVESGGGLVQPGGSLRVSCAASGFTFSTYYMSWVR | 157 | QAVVTQEPSLSVSPGGTVTLTCGLSSGSVTTSNYPGWF | 158 |
| | QAPGKGLEWVSDIRTDGGTYYADSVKGRFTMSRDNAKN | | QQTPGQAPRTLIYNTNSRHSGVPSRFSGSISGNKAALT | |
| | TLYLQMNSLKPEDTALYYCARTRIFPSGYDYWGQGTQV | | IMGAQPEDEADYYCSLYPGSTTVFGGGTHLTVL | |
| | TVSS | | | |
| 71C3 | QLQLVESGGGLVQPGGSLRLSCAASGFTFSSHAMSWVR | 159 | SYELTQPSALSVTLGQTAKITCQGGSLGSSYAHWYQQK | 160 |
| | QAPGKGLEWVSAINSGGGSTSYADSVKGRFTISRDNAK | | PGQAPVLVIYDDDSRPSGIPERFSGSSSGGTATLTISG | |
| | NTLYLQMNSLKPEDTAVYYCAKELRFDLARYTDYEAWD | | AQAEDEGDYYCQSADSSGNAAVFGGGTHLTVL | |
| | YWGQGTQVTVSS | | | |
| 71D4 | ELQLVESGGGLVQPGGSLRLSCAASGFTFSGYGMSWVR | 161 | SSALTQPSALSVTLGQTAKITCQGGSLGSSYAHWYQQK | 162 |
| | QAPGKGLEWVSDINSGGGSTSYADSVKGRFTISRDNAK | | PGQAPVLVIYDDDSRPSGIPERFSGSSSGGTATLTISG | |
| | NTLYLQMNSLKPEDTAVYYCAKDMRLYLARYNDYEAWD | | AQAEDEGDYYCQSADSSGNAAVFGGGTHLTVL | |
| | YWGQGTQVTVSS | | | |
| 71D6 | ELQLVESGGGLVQPGGSLRLSCAASGFTFSSYGMSWVR | 163 | QPVLNQPSALSVTLGQTAKITCQGGSLGARYAHWYQQK | 164 |
| | QAPGKGLEWVSAINSYGGSTSYADSVKGRFTISRDNAK | | PGQAPVLVIYDDDSRPSGIPERFSGSSSGGTATLTISG | |
| | NTLYLQMNSLKPEDTAVYYCAKEVRADLSRYNDYESYD | | AQAEDEGDYYCQSADSSGSVFGGGTHLTVL | |
| | YWGQGTQVTVSS | | | |
| 71A3 | EVQLVESGGGLVQPGGSLRLSCAASGFSFKDYDITWVR | 165 | SYELTQPSALSVTLGQTAKITCQGGSLGSSYAHWYQQK | 166 |
| | QAPGKGLEWVSTITSRSGSTSYVDSVKGRFTISGDNAK | | PGQAPVLVIYDDDSRPSGIPERFSGSSSGGTATLTISG | |
| | NTLYLQMNSLKPEDTAVYYCAKVYATTWDVGPLGYGMD | | AQAEDEGDYYCQSADSSGNAAVFGGGTHLTVL | |
| | YWGKGTLVTVSS | | | |
| 71G2 | EVQLQESGGGLVQPGGSLRLSCAASGFTFSIYDMSWVR | 167 | SSALTQPSALSVSLGQTARITCQGGSLGSSYAHWYQQK | 168 |
| | QAPGKGLEWVSTINSDGSSTSYVDSVKGRFTISRDNAK | | PGQAPVLVIYGDDSRPSGIPERFSGSSSGGTATLTISG | |
| | NTLYLQMNSLKPEDTAVYYCAKVYGSTWDVGPMGYGMD | | AQAEDEDDYYCQSTDSSGNTVFGGGTRLTVL | |
| | YWGKGTLVTVSS | | | |
| 76G7 | QVQLVESGGNLVQPGGSLRLSCAASGFTFSNYYMSWVR | 169 | QAGLTQPPSVSGSPGKTVTISCAGNSSDVGYGNYVSWY | 170 |
| | QAPGKGLEWVSDIYSDGSTTWYSDSVKGRFTISRDNAK | | QQFPGMAPKLLIYLVNKRASGITDRFSGSKSGNTASLT | |
| | NTLSLQMNSLKSEDTAVYYCARVKIYPGGYDAWGQGTQ | | ISGLQSEDEADYYCASYTGSNNIVFGGGTHLTVL | |
| | VTVSS | | | |
| 71G12 | QVQLQESGGDLVQPGGSLRVSCVVSGFTFSRYYMSWVR | 171 | EIVLTQSPSSVTASVGGKVTINCKSSQSVFIASNQKTY | 172 |
| | QAPGKGLEWVSSIDSYGYSTYYTDSVKGRFTISRDNAK | | LNWYQQRPGQSPRLVISYASTRESGIPDRFSGSGSTTD | |
| | NTLYLQMNSLKPEDTALYYCARAKTTWSYDYWGQGTQV | | FTLTISSVQPEDAAVYYCQQAYSHPTFGQGTKVELK | |
| | TVSS | | | |
| 74C8 | EVQLVESGGGLVQPGGSLRLSCAASGFTFRNYHMSWVR | 173 | QTVVTQEPSLSVSPGGTVTLTCGLSSGSVTTSNYPGWF | 174 |
| | QVPGKGFEWISDINSAGGSTYYADSVKGRFTISRDNAK | | QQTPGQAPRTLIYNTNSRHSGVPSRFSGSISGNKAALT | |
| | NTLYLEMNSLKPEDTALYYCARVNVWGVNYWGKGTLVS | | ITGAQPEDEADYYCSLYPGSYTNVFGGGTHLTVL | |
| | VSS | | | |
| 72F8 | ELQLVESGGGLVQPGGSLRLSCAASGFTFSNYVMSWVR | 175 | QSALTQPPSLSASPGSSVRLTCTLSSGNNIGSYDISWY | 176 |
| | QAPGKGLEWVSDTNSGGSTSYADSVKGRFTISRDNAKN | | QQKAGSPPRYLLNYYTDSRKHQDSGVPSRFSGSKDASA | |
| | TLYLQMNSLKPEDTALYYCARSFFYGMNYWGKGTQVTV | | NAGLLLISGLQPEDEADYYCSAYKSGSYRWVFGGGTHV | |
| | SS | | TVL | |

TABLE 6
Variable domain nucleotide sequences of Fabs binding to both human and mouse MET.

| | | SEQ ID | | SEQ ID |
|---|---|---|---|---|
| Clone | VH | NO. | VL | NO. |
| 76H10 | CAGTTGCAGCTGGTGGAGTCTGGGGGAGGCTTGGTGCA | 177 | CAGGCTGTGGTGACCCAGGAGCCGTCCCTGTCAGTGTC | 178 |
| | GCCTGGGGGGTCTCTGAGAGTTTCCTGTACAGCCTCTG | | TCCAGGAGGGACGGTCACACTCACCTGCGGCCTCAGCT | |
| | GATTCACCTTCAATACCTACTACATGACCTGGGTCCGC | | CTGGGTCTGTCACTACCAGTAACTACCCTGGTTGGTTC | |
| | CAGGCTCCAGGGAAGGGGCTCGAGTGGGTCTCAGATAT | | CAGCAGACACCGGGCCAGGCTCCACGCACTCTTATCTA | |
| | TAATAGTGGTGGTGGTACATACTATGCAGACTCCGTGA | | CAACACAAACAACCGCCACTCTGGGGTCCCCAGTCGCT | |
| | AGGGCCGATTCACCATCTCCAGAGACAACGCCAAGAAC | | TCTCCGGATCCATCTCTGGGAACAAAGCCGCCCTCACC | |
| | ACGCTATATCTGCAAATGAACAGCCTGAAACCTGAGGA | | ATCACGGGGGCCCAGCCCGAGGACGAGGCCGACTATTA | |
| | CACGGCCCTGTATTACTGTGTAAGAGTTCGTATTTGGC | | CTGTTCTCTATATACTGGCAGTTACACTACTGTGTTCG | |
| | CAGTGGGATATGACTACTGGGGCCAGGGGACCCAGGTC | | GCGGAGGGACCCATCTGACCGTCCTG | |
| | ACCGTTTCCTCA | | | |
| 71G3 | CAGGTGCAGCTCGTGGAGTCTGGGGGAGGCTTGGTGCA | 179 | CAGGCTGTGGTGACCCAGGAGCCGTCCCTGTCAGTGTC | 180 |
| | GCCTGGGGGGTCTCTGAGAGTCTCCTGTGCAGCCTCTG | | TCCAGGAGGGACGGTCACACTCACCTGCGGCCTCAGCT | |
| | GATTCACCTTCAGTACCTACTACATGAGCTGGGTCCGC | | CTGGGTCTGTCACTACCAGTAACTACCCTGGTTGGTTC | |
| | CAGGCTCCAGGGAAGGGGCTCGAGTGGGTCTCAGATAT | | CAGCAGACACCAGGCCAGGCTCCGCGCACTCTTATCTA | |
| | TCGTACTGATGGTGGCACATACTATGCAGACTCCGTGA | | CAACACAAACAGCCGCCACTCTGGGGTCCCCAGTCGCT | |
| | AGGGCCGATTCACCATGTCCAGAGACAACGCCAAGAAC | | TCTCCGGATCCATCTCTGGGAACAAAGCCGCCCTCACC | |
| | ACGCTGTATCTACAAATGAACAGCCTGAAACCTGAGGA | | ATCATGGGGGCCCAGCCCGAGGACGAGGCCGACTATTA | |
| | CACGGCCCTGTATTACTGTGCAAGAACTCGAATTTTCC | | CTGTTCTCTGTACCCTGGTAGTACCACTGTGTTCGGCG | |
| | CCTCGGGGTATGACTACTGGGGCCAGGGGACCCAGGTC | | GAGGGACCCATCTGACCGTCCTG | |
| | ACCGTCTCCTCA | | | |
| 71C3 | CAGTTGCAGCTGGTGGAGTCTGGGGGAGGCTTGGTGCA | 181 | TCCTATGAGCTGACTCAGCCCTCCGCGCTGTCCGTAAC | 182 |
| | GCCTGGGGGGTCTCTGAGACTCTCCTGTGCAGCCTCTG | | CTTGGGACAGACGGCCAAGATCACCTGCCAAGGTGGCA | |
| | GATTCACCTTCAGTAGCCATGCCATGAGCTGGGTCCGC | | GCTTAGGTAGCAGTTATGCTCACTGGTACCAGCAGAAG | |
| | CAGGCTCCAGGAAAGGGGCTCGAGTGGGTCTCAGCTAT | | CCAGGCCAGGCCCCTGTGCTGGTCATCTATGATGATGA | |
| | TAATAGTGGTGGTGGTAGCACAAGCTATGCAGACTCCG | | CAGCAGGCCCTCAGGGATCCCTGAGCGGTTCTCTGGCT | |
| | TGAAGGGCCGATTCACCATCTCCAGAGACAACGCCAAG | | CCAGCTCTGGGGGCACAGCCACCCTGACCATCAGCGGG | |
| | AACACGCTGTACCTGCAAATGAACAGCCTGAAACCTGA | | GCCCAGGCCGAGGACGAGGGTGACTATTACTGTCAGTC | |
| | GGACACGGCCGTGTATTACTGTGCAAAAGAGCTGAGAT | | AGCAGACAGCAGTGGTAATGCTGCTGTGTTCGGCGGAG | |
| | TCGACCTAGCAAGGTATACCGACTATGAGGCCTGGGAC | | GGACCCATCTGACCGTCCTG | |
| | TACTGGGGCCAGGGGACCCAGGTCACCGTCTCCTCA | | | |
| 71D4 | GAGTTGCAGCTGGTGGAGTCTGGGGGAGGCTTGGTGCA | 183 | TCCTCTGCACTGACTCAGCCCTCCGCGCTGTCCGTAAC | 184 |
| | GCCTGGGGGGTCTCTGAGACTCTCCTGTGCAGCCTCTG | | CTTGGGACAGACGGCCAAGATCACCTGCCAAGGTGGCA | |
| | GATTCACCTTCAGTGGCTATGGCATGAGCTGGGTCCGC | | GCTTAGGTAGCAGTTATGCTCACTGGTACCAGCAGAAG | |
| | CAGGCTCCAGGAAAGGGGCTCGAGTGGGTCTCAGATAT | | CCAGGCCAGGCCCCTGTGCTGGTCATCTATGATGATGA | |
| | TAATAGTGGTGGTGGTAGCACAAGCTATGCAGACTCCG | | CAGCAGGCCCTCAGGGATCCCTGAGCGGTTCTCTGGCT | |
| | TGAAGGGCCGATTCACCATCTCCAGAGACAACGCCAAG | | CCAGCTCTGGGGGCACAGCCACCCTGACCATCAGCGGG | |
| | AACACGCTGTATCTGCAAATGAACAGCCTGAAACCTGA | | GCCCAGGCCGAGGACGAGGGTGACTATTACTGTCAGTC | |
| | GGACACGGCCGTGTATTACTGTGCAAAAGATATGAGAT | | AGCAGACAGCAGTGGTAATGCTGCTGTGTTCGGCGGAG | |
| | TATACCTAGCAAGGTATAACGACTATGAGGCCTGGGAC | | GGACCCATCTGACCGTCCTG | |
| | TACTGGGGCCAGGGGACCCAGGTCACCGTCTCCTCA | | | |
| 71D6 | GAGTTGCAGCTGGTGGAGTCTGGGGGAGGCTTGGTGCA | 185 | CAGCCGGTGCTGAATCAGCCCTCCGCGCTGTCCGTAAC | 186 |
| | GCCTGGGGGGTCTCTGAGACTCTCCTGTGCAGCCTCTG | | CTTGGGACAGACGGCCAAGATCACCTGCCAAGGTGGCA | |
| | GATTCACCTTCAGTAGCTATGGCATGAGCTGGGTCCGC | | GCTTAGGTGCGCGTTATGCTCACTGGTACCAGCAGAAG | |
| | CAGGCTCCAGGAAAGGGGCTCGAGTGGGTCTCAGCTAT | | CCAGGCCAGGCCCCTGTGCTGGTCATCTATGATGATGA | |
| | TAATAGTTATGGTGGTAGCACAAGCTATGCAGACTCCG | | CAGCAGGCCCTCAGGGATCCCTGAGCGGTTCTCTGGCT | |
| | TGAAGGGCCGATTCACCATCTCCAGAGACAACGCCAAG | | CCAGCTCTGGGGGCACAGCCACCCTGACCATCAGCGGG | |
| | AACACGCTGTATCTGCAAATGAACAGCCTGAAACCTGA | | GCCCAGGCCGAGGACGAGGGTGACTATTACTGTCAGTC | |
| | GGACACGGCCGTGTATTACTGTGCAAAAGAAGTGCGGG | | AGCAGACAGCAGTGGTTCTGTGTTCGGCGGAGGGACCC | |
| | CCGACCTAAGCCGCTATAACGACTATGAGTCGTATGAC | | ATCTGACCGTCCTG | |
| | TACTGGGGCCAGGGGACCCAGGTCACCGTCTCCTCA | | | |
| 71A3 | GAGGTGCAGCTCGTGGAGTCTGGGGGAGGCTTGGTGCA | 187 | TCCTATGAGCTGACTCAGCCCTCCGCGCTGTCCGTAAC | 188 |
| | GCCTGGGGGGTCTCTGAGACTCTCCTGTGCAGCCTCTG | | CTTGGGACAGACGGCCAAGATCACCTGCCAAGGTGGCA | |
| | GATTCAGCTTCAAGGACTATGACATAACCTGGGTCCGC | | GCTTAGGTAGCAGTTATGCTCACTGGTACCAGCAGAAG | |
| | CAGGCTCCGGGAAAGGGGCTCGAGTGGGTCTCAACTAT | | CCAGGCCAGGCCCCTGTGCTGGTCATCTATGATGATGA | |
| | TACTAGTCGTAGTGGTAGCACAAGCTATGTAGACTCCG | | CAGCAGGCCCTCAGGGATCCCTGAGCGGTTCTCTGGCT | |
| | TAAAGGGCCGATTCACCATCTCCGGAGACAACGCCAAG | | CCAGCTCTGGGGGCACAGCCACCCTGACCATCAGCGGG | |
| | AACACGCTGTATCTGCAAATGAACAGCCTGAAACCTGA | | GCCCAGGCCGAGGACGAGGGTGACTATTACTGTCAGTC | |
| | GGACACGGCCGTGTATTACTGTGCAAAAGTTTACGCGA | | AGCAGACAGCAGTGGTAATGCTGCTGTGTTCGGCGGAG | |
| | CTACCTGGGACGTCGGCCCTCTGGGCTACGGCATGGAC | | GGACCCATCTGACCGTCCTG | |
| | TACTGGGGCAAGGGGACCCTGGTCACCGTCTCCTCA | | | |
| 71G2 | GAGGTGCAGCTGCAGGAGTCGGGGGGAGGCTTGGTGCA | 189 | TCCTCTGCACTGACTCAGCCCTCCGCGCTGTCCGTGTC | 190 |
| | GCCTGGGGGGTCTCTGAGACTCTCCTGTGCAGCCTCTG | | CTTGGGACAGACGGCCAGGATCACCTGCCAAGGTGGCA | |
| | GATTCACCTTCAGTATATATGACATGAGCTGGGTCCGC | | GCTTAGGTAGCAGTTATGCTCACTGGTACCAGCAGAAG | |
| | CAGGCTCCAGGAAAGGGGCTCGAGTGGGTCTCAACTAT | | CCAGGCCAGGCCCCTGTGCTGGTCATCTATGGTGATGA | |
| | TAATAGTGATGGTAGTAGCACAAGCTATGTAGACTCCG | | CAGCAGGCCCTCAGGGATCCCTGAGCGGTTCTCTGGCT | |
| | TGAAGGGCCGATTCACCATCTCCAGAGACAACGCCAAG | | CCAGCTCTGGGGGCACAGCCACCCTGACCATCAGCGGG | |
| | AACACGCTGTATCTGCAAATGAACAGCCTGAAACCTGA | | GCCCAGGCCGAGGACGAGGATGACTATTACTGTCAGTC | |
| | GGACACGGCCGTGTATTACTGTGCGAAAGTTTACGGTA | | AACAGACAGCAGTGGTAATACTGTGTTCGGCGGAGGGA | |
| | GTACCTGGGACGTCGGCCCTATGGGCTACGGCATGGAC | | CCCGACTGACCGTCCTG | |
| | TACTGGGGCAAAGGGACCCTGGTCACTGTCTCCTCA | | | |
| 76G7 | CAGGTGCAGCTGGTGGAGTCTGGGGGAAACTTGGTGCA | 191 | CAGGCAGGGCTGACTCAGCCTCCCTCCGTGTCTGGGTC | 192 |
| | GCCTGGGGGTTCTCTGAGACTCTCCTGTGCAGCCTCTG | | TCCAGGAAAGACGGTCACCATCTCCTGTGCAGGAAACA | |
| | GATTCACCTTCAGTAACTACTACATGAGCTGGGTCCGC | | GCAGTGATGTTGGGTATGGAAACTATGTCTCCTGGTAC | |
| | CAGGCTCCAGGGAAGGGGCTGGAATGGGTGTCCGATAT | | CAGCAGTTCCCAGGAATGGCCCCCAAACTCCTGATATA | |
| | TTATAGTGACGGTAGTACCACATGGTATTCAGACTCCG | | TCTCGTCAATAAACGGGCCTCAGGGATCACTGATCGCT | |
| | TCAAGGGCCGATTCACCATCTCCAGAGACAACGCCAAG | | TCTCTGGCTCCAAGTCAGGCAACACGGCCTCCCTGACC | |
| | AACACGCTGTCTCTGCAAATGAACAGTCTGAAATCTGA | | ATCTCTGGGCTCCAGTCTGAGGACGAGGCTGATTATTA | |
| | GGACACGGCCGTCTATTACTGTGCGCGCGTGAAGATCT | | CTGTGCCTCATATACAGGTAGCAACAATATCGTGTTCG | |
| | ATCCGGGGGGGTATGACGCCTGGGGCCAGGGGACCCAG | | GCGGAGGGACCCATCTAACCGTCCTC | |
| | GTCACCGTCTCCTCA | | | |
| 71G12 | CAGGTGCAGCTGCAGGAGTCGGGGGGAGACTTGGTGCA | 193 | GAAATTGTGTTGACGCAGTCTCCCAGCTCCGTGACTGC | 194 |
| | GCCTGGGGGGTCTCTGAGAGTCTCCTGTGTAGTCTCTG | | ATCTGTAGGAGGGAAGGTCACTATCAACTGTAAGTCCA | |
| | GATTCACCTTCAGTCGCTACTACATGAGCTGGGTCCGC | | GCCAGAGCGTCTTCATAGCTTCTAATCAGAAAACCTAC | |
| | CAGGCTCCAGGGAAGGGGCTCGAGTGGGTCTCATCTAT | | TTAAACTGGTACCAGCAGAGACCTGGACAGTCTCCGAG | |
| | TGATAGTTATGGTTACAGCACATACTATACAGACTCCG | | GTTGGTCATCAGCTATGCGTCCACCCGTGAATCGGGGA | |
| | TGAAGGGCCGATTCACCATCTCCAGAGACAACGCCAAG | | TCCCTGATCGATTCAGCGGCAGTGGGTCCACAACAGAT | |
| | AACACGCTGTATCTGCAAATGAACAGCCTGAAACCTGA | | TTCACTCTCACGATCAGCAGTGTCCAGCCTGAAGATGC | |
| | GGACACGGCCCTGTATTACTGTGCAAGAGCGAAAACGA | | GGCCGTGTATTACTGTCAGCAGGCTTATAGCCATCCAA | |
| | CTTGGAGTTATGACTACTGGGGCCAGGGGACCCAGGTC | | CGTTCGGCCAGGGGACCAAGGTGGAACTCAAA | |
| | ACCGTCTCCTCA | | | |
| 74C8 | GAGGTGCAGCTCGTGGAGTCTGGGGGAGGCTTGGTGCA | 195 | CAGACTGTGGTGACTCAGGAGCCGTCCCTGTCAGTGTC | 196 |
| | ACCTGGGGGTTCTCTGAGACTCTCCTGTGCAGCCTCTG | | TCCAGGAGGGACGGTCACACTCACCTGCGGCCTCAGCT | |
| | GATTCACCTTCAGGAATTACCACATGAGTTGGGTCCGC | | CTGGGTCTGTCACTACCAGTAACTACCCTGGTTGGTTC | |
| | CAGGTTCCAGGGAAGGGGTTCGAGTGGATCTCAGATAT | | CAGCAGACACCAGGCCAGGCTCCACGCACTCTTATCTA | |
| | TAATAGTGCAGGTGGTAGCACATACTATGCAGACTCCG | | CAACACAAACAGCCGCCACTCTGGGGTCCCCAGTCGCT | |
| | TGAAGGGCCGATTCACCATCTCCAGAGACAACGCCAAG | | TCTCCGGATCCATCTCTGGGAACAAAGCCGCCCTCACC | |
| | AACACGCTGTATCTGGAAATGAACAGCCTGAAACCTGA | | ATCACGGGGGCCCAGCCCGAGGACGAGGCCGACTATTA | |
| | GGACACGGCCCTGTATTACTGTGCAAGAGTCAACGTCT | | CTGTTCTCTGTACCCTGGTAGTTACACTAATGTGTTCG | |
| | GGGGGGTGAACTACTGGGGCAAAGGGACCCTGGTCAGC | | GCGGAGGGACCCATCTGACCGTCCTG | |
| | GTCTCCTCA | | | |
| 72F8 | GAGTTGCAGCTGGTGGAGTCTGGGGGAGGCTTGGTGCA | 197 | CAGTCTGCCCTGACTCAGCCGCCCTCCCTCTCTGCATC | 198 |
| | GCCTGgGGGGTCTCTGAGACTCTcCTGTGCAGCCTCTG | | TCCGGGATCATCTGTCAGACTCACCTGCACCCTGAGCA | |
| | GATTCACCTTCAGCAACTATGTCATGAGCTGGGTCCGC | | GTGGAAACAATATTGGCAGCTATGACATAAGTTGGTAC | |
| | CAGGCTCCAGGAAAGGGGCTCGAGTGGGTCTCAGATAC | | CAGCAGAAGGCAGGGAGCCCTCCCCGGTACCTCCTGAA | |
| | TAATAGTGGTGGTAGCACAAGCTATGCAGACTCCGTGA | | CTACTACACCGACTCACGCAAGCACCAGGACTCCGGGG | |
| | AGGGCCGATTCACCATCTCTAGAGACAACGCCAAGAAC | | TCCCGAGCCGCTTCTCTGGGTCCAAAGATGCCTCGGCC | |
| | ACGCTGTATTTGCAAATGAACAGCCTGAAACCTGAGGA | | AACGCAGGGCTTCTGCTCATCTCTGGGCTTCAGCCCGA | |
| | CACGGCATTGTATTACTGTGCGAGATCATTTTTCTACG | | GGACGAGGCTGACTATTACTGTTCTGCATACAAGAGTG | |
| | GCATGAACTACTGGGGCAAAGGGACCCAGGTCACCGTG | | GTTCTTACCGTTGGGTGTTCGGCGGAGGGACGCACGTG | |
| | TCCTCA | | ACCGTCCTG | |

The various Fab families and their ability to bind human and mouse MET are shown in Table 7.

TABLE 7
Fabs binding to both human MET (hMET) and mouse MET (mMET).
Fabs are grouped in families based on their VH CDR3 sequence.
Binding of Fabs to human and mouse MET ECD was determined
by Surface Plasmon Resonance (SPR) and by ELISA. SPR values
represent the koff (s⁻¹). ELISA values represent the Optical
Density (OD) at 450 nm (AU, arbitrary units). Both SPR and
ELISA were performed using crude periplasmic extracts.
Fab concentration in the extract was not determined.
Values are the mean of three independent measurements.

| | | | SPR (K_{off}; s⁻¹) | | ELISA (OD₄₅₀; AU) | |
|---|---|---|---|---|---|---|
| Fab | VH | VL | hMET | mMET | hMET | mMET |
| 76H10 | VH 1 | Lambda | 5.68E−03 | 5.44E−03 | 3.704 | 3.697 |
| 71G3 | VH 2 | Lambda | 1.42E−03 | 1.41E−03 | 3.462 | 3.443 |
| 71D6 | VH 3a | Lambda | 2.94E−03 | 2.67E−03 | 3.261 | 3.072 |
| 71C3 | VH 3b | Lambda | 2.25E−03 | 2.58E−03 | 1.650 | 1.643 |
| 71D4 | VH 3c | Lambda | 2.17E−03 | 2.38E−03 | 0.311 | 0.307 |
| 71A3 | VH 4 | Lambda | 4.92E−03 | 4.74E−03 | 0.581 | 0.524 |
| 71G2 | VH 4 | Lambda | 1.21E−03 | 1.48E−03 | 0.561 | 0.543 |
| 76G7 | VH 5 | Lambda | 4.32E−03 | 4.07E−03 | 3.199 | 3.075 |
| 71G12 | VH 6 | Kappa | 2.28E−03 | 2.55E−03 | 0.450 | 0.420 |
| 74C8 | VH 9 | Lambda | 3.48E−03 | 3.70E−03 | 2.976 | 2.924 |
| 72F8 | VH 10 | Lambda | 4.96E−03 | 4.58E−03 | 3.379 | 3.085 |

### Example 3: Chimerization of Fabs into mAbs

The cDNAs encoding the VH and VL (K or A) domains of selected Fab fragments were engineered into two separate pUPE mammalian expression vectors (U-protein Express) containing the cDNAs encoding CH1, CH2 and CH3 of human IgG1 or the human CL (K or A), respectively.

Production (by transient transfection of mammalian cells) and purification (by protein A affinity chromatography) of the resulting chimeric llama-human IgG1 molecules was outsourced to U-protein Express. Binding of chimeric mAbs to MET was determined by ELISA using hMET or mMET ECD in solid phase and increasing concentrations of antibodies (0-20 nM) in solution. Binding was revealed using HRP-conjugated anti-human Fc antibodies (Jackson Immuno Research Laboratories). This analysis revealed that all chimeric llama-human antibodies bound to human and mouse MET with picomolar affinity, displaying an EC₅₀ comprised between 0.06 nM and 0.3 nM. Binding capacity (E_{MAX}) varied from antibody to antibody, possibly due to partial epitope exposure in the immobilized antigen, but was similar in the human and mouse setting. EC₅₀ and E_{MAX} values are shown in Table 9.

TABLE 9
Binding of chimeric mAbs to human and mouse
MET as determined by ELISA using immobilized
MET ECD in solid phase and increasing
concentrations (0-20 nM) of antibodies in solution.

| | hMET | | mMET | |
|---|---|---|---|---|
| mAb | EC₅₀ | E_{MAX} | EC₅₀ | E_{MAX} |
| 76H10 | 0.090 | 2.669 | 0.062 | 2.662 |
| 71G3 | 0.067 | 2.835 | 0.057 | 2.977 |
| 71D6 | 0.026 | 2.079 | 0.049 | 2.009 |
| 71C3 | 0.203 | 2.460 | 0.293 | 2.238 |
| 71D4 | 0.207 | 1.428 | 0.274 | 1.170 |
| 71A3 | 0.229 | 2.401 | 0.176 | 2.730 |
| 71G2 | 0.112 | 3.094 | 0.101 | 3.168 |
| 76G7 | 0.128 | 2.622 | 0.103 | 2.776 |
| 71G12 | 0.106 | 3.076 | 0.127 | 2.973 |
| 74C8 | 0.090 | 0.994 | 0.116 | 0.896 |
| 72F8 | 0.064 | 2.779 | 0.048 | 2.903 |

EC₅₀ values are expressed as nMol/L.
E_{MAX} values are expressed as Optical Density (OD) at 450 nm (AU, arbitrary units).

We also analysed whether chimeric anti-MET antibodies bound to native human and mouse MET in living cells. To this end, increasing concentrations of antibodies (0-100 nM) were incubated with A549 human lung carcinoma cells (American Type Culture Collection) or MLP29 mouse liver precursor cells (a gift of Prof. Enzo Medico, University of Torino, Strada Provinciale 142 km 3.95, Candiolo, Torino, Italy; Medico et al., Mol Biol Cell 7, 495-504, 1996), which both express physiological levels of MET. Antibody binding to cells was analysed by flow cytometry using phycoerythrin-conjugated anti-human IgG1 antibodies (eBioscience) and a CyAn ADP analyser (Beckman Coulter). As a positive control for human MET binding, we used a commercial mouse anti-human MET antibody (R&D Systems) and phycoerythrin-conjugated anti-mouse IgG1 antibodies (eBioscience). As a positive control for mouse MET binding we used a commercial goat anti-mouse MET antibody (R&D Systems) and phycoerythrin-conjugated anti-goat IgG1 antibodies (eBioscience). All antibodies displayed dose-dependent binding to both human and mouse cells with an EC₅₀ varying between 0.2 nM and 2.5 nM. Consistent with the data obtained in ELISA, maximal binding (E_{MAX}) varied depending on antibody, but was similar in human and mouse cells. These results indicate that the chimeric llama-human antibodies recognize membrane-bound MET in its native conformation in both human and mouse cellular systems. EC₅₀ and E_{MAX} values are shown in Table 10.

TABLE 10
Binding of chimeric mAbs to human and mouse
cells as determined by flow cytometry using
increasing concentrations (0-50 nM) of antibodies.

| | Human cells (A549) | | Mouse cells (MLP29) | |
|---|---|---|---|---|
| mAb | EC₅₀ | E_{MAX} | EC₅₀ | E_{MAX} |
| 76H10 | 2.345 | 130.2 | 1.603 | 124.3 |
| 71G3 | 0.296 | 116.9 | 0.214 | 116.2 |
| 71D6 | 0.259 | 112.7 | 0.383 | 121.2 |
| 71C3 | 0.572 | 106.5 | 0.585 | 115.1 |
| 71D4 | 0.371 | 107.2 | 0.498 | 94.8 |
| 71A3 | 0.514 | 160.8 | 0.811 | 144.2 |
| 71G2 | 0.604 | 144.4 | 0.688 | 129.9 |
| 76G7 | 2.298 | 121.2 | 2.371 | 114.8 |
| 71G12 | 2.291 | 109.9 | 2.539 | 121.2 |
| 74C8 | 0.235 | 85.7 | 0.208 | 73.8 |
| 72F8 | 0.371 | 156.3 | 0.359 | 171.6 |

EC₅₀ values are expressed as nMol/L.
E_{MAX} values are expressed as % relative to control.

### Example 4: Receptor Regions Responsible for Antibody Binding

In order to map the receptor regions recognized by antibodies binding to both human and mouse MET (herein after referred to as human/mouse equivalent anti-MET antibodies), we measured their ability to bind to a panel of engineered proteins derived from human MET generated as described (Basilico et al, J Biol. Chem. 283, 21267-21227, 2008). This panel included: the entire MET ECD (Decoy MET); a MET ECD lacking IPT domains 3 and 4 (SEMA-PSI-IPT 1-2); a MET ECD lacking IPT domains 1-4 (SEMA-PSI); the isolated SEMA domain (SEMA); a fragment containing IPT domains 3 and 4 (IPT 3-4). Engineered MET proteins were immobilized in solid phase and exposed to increasing concentrations of chimeric antibodies (0-50 nM) in solution. Binding was revealed using HRP-conjugated anti-human Fc antibodies (Jackson Immuno Research Laboratories). As shown in Table 11, this analysis revealed that 7 mAbs recognize an epitope within the SEMA domain, while the other 4 recognize an epitope within the PSI domain.

TABLE 11
Binding of human/mouse equivalent anti-MET antibodies to the panel
of MET deletion mutants. The MET domain responsible for antibody
binding is indicated in the last column to the right.

| | | SEMA- | | | | |
|---|---|---|---|---|---|---|
| | Decoy | PSI-IPT | SEMA- | | IPT | Binding |
| mAb | MET | 1-2 | PSI | SEMA | 3-4 | domain |
| 76H10 | + | + | + | − | − | PSI |
| 71G3 | + | + | + | − | − | PSI |
| 71D6 | + | + | + | + | − | SEMA |
| 71C3 | + | + | + | + | − | SEMA |
| 71D4 | + | + | + | + | − | SEMA |
| 71A3 | + | + | + | + | − | SEMA |
| 71G2 | + | + | + | + | − | SEMA |
| 76G7 | + | + | + | − | − | PSI |
| 71G12 | + | + | + | − | − | PSI |
| 74C8 | + | + | + | + | − | SEMA |
| 72F8 | + | + | + | + | − | SEMA |

To more finely map the regions of MET responsible for antibody binding, we exploited the absence of cross-reactivity between our antibodies and llama MET (the organism used for generating these immunoglobulins). To this end, we generated a series of llama-human and human-llama chimeric MET proteins spanning the entire MET ECD as described (Basilico et al., J Clin Invest. 124, 3172-3186, 2014). Chimeras were immobilized in solid phase and then exposed to increasing concentrations of mAbs (0-20 nM) in solution. Binding was revealed using HRP-conjugated anti-human Fc antibodies (Jackson Immuno Research Laboratories). This analysis unveiled that 5 SEMA-binding mAbs (71D6, 71C3, 71D4, 71A3, 71G2) recognize an epitope localized between aa 314-372 of human MET, a region that corresponds to blades 4-5 of the 7-bladed SEMA β-propeller (Stamos et al., EMBO J. 23, 2325-2335, 2004). The other 2 SEMA-binding mAbs (74C8, 72F8) recognize an epitope localized between aa 123-223 and 224-311, respectively, corresponding to blades 1-3 and 1-4 of the SEMA β-propeller. The PSI-binding mAbs (76H10, 71G3, 76G7, 71G12) did not appear to display any significant binding to any of the two PSI chimeras. Considering the results presented in Table 11, these antibodies probably recognize an epitope localized between aa 546 and 562 of human MET. These results are summarized in Table 12.

TABLE 12
Mapping of the epitopes recognized by human/mouse equivalent anti-MET antibodies
as determined by ELISA. Human MET ECD (hMET) or llama MET ECD (lMET) as
well as the llama-human MET chimeric proteins (CH1-7) were immobilized
in solid phase and then exposed to increasing concentrations of mAbs.

| | | | | | | | | | | Epitope |
|---|---|---|---|---|---|---|---|---|---|---|
| mAb | hMET | lMET | CH1 | CH2 | CH3 | CH4 | CH5 | CH6 | CH7 | (aa) |
| 76H10 | + | − | + | + | + | + | + | − | − | 546-562 |
| 71G3 | + | − | + | + | + | + | + | − | − | 546-562 |
| 71D6 | + | − | + | + | + | − | − | + | + | 314-372 |
| 71C3 | + | − | + | + | + | − | − | + | + | 314-372 |
| 71D4 | + | − | + | + | + | − | − | + | + | 314-372 |
| 71A3 | + | − | + | + | + | − | − | + | + | 314-372 |
| 71G2 | + | − | + | + | + | − | − | + | + | 314-372 |
| 76G7 | + | − | + | + | + | + | + | − | − | 546-562 |
| 71G12 | + | − | + | + | + | + | + | − | − | 546-562 |
| 74C8 | + | − | + | − | − | − | − | + | + | 123-223 |
| 72F8 | + | − | + | + | − | − | − | + | + | 224-311 |

### Example 5: HGF Competition Assays

The above analysis suggests that the epitopes recognized by some of the human/mouse equivalent anti-MET antibodies may overlap with those engaged by HGF when binding to MET (Stamos et al., EMBO J. 23, 2325-2335, 2004; Merchant et al., Proc Natl Acad Sci USA 110, E2987-2996, 2013; Basilico et al., J Clin Invest. 124, 3172-3186, 2014). To investigate along this line, we tested the competition between mAbs and HGF by ELISA. Recombinant human and mouse HGF (R&D Systems) were biotinylated at the N-terminus using NHS-LC-biotin (Thermo Scientific). MET-Fc protein, either human or mouse (R&D Systems), was immobilized in solid phase and then exposed to 0.3 nM biotinylated HGF, either human or mouse, in the presence of increasing concentrations of antibodies (0-120 nM). HGF binding to MET was revealed using HRP-conjugated streptavidin (Sigma-Aldrich). As shown in Table 13, this analysis allowed to divide human/mouse equivalent anti-MET mAbs into two groups: full HGF competitors (71D6, 71C3, 71D4, 71A3, 71G2), and partial HGF competitors (76H10, 71G3, 76G7, 71G12, 74C8, 72F8).

TABLE 13
Ability of human/mouse equivalent
anti-MET antibodies to compete with HGF
for binding to MET as determined by ELISA.

| | hHGF on hMET | | mHGF on mMET | |
|---|---|---|---|---|
| mAb | IC₅₀ (nM) | I_{MAX} (%) | IC₅₀ (nM) | I_{MAX} (%) |
| 76H10 | 1.86 | 64.22 | 2.01 | 62.71 |
| 71G3 | 0.49 | 63.16 | 0.53 | 62.87 |
| 71D6 | 0.29 | 98.34 | 0.34 | 90.54 |
| 71C3 | 1.42 | 93.64 | 1.56 | 89.23 |
| 71D4 | 0.34 | 95.62 | 0.40 | 91.34 |
| 71A3 | 0.51 | 93.37 | 0.54 | 87.74 |
| 71G2 | 0.23 | 97.84 | 0.26 | 91.86 |
| 76G7 | 1.47 | 69.42 | 1.56 | 62.52 |
| 71G12 | 3.87 | 51.39 | 4.05 | 50.67 |
| 74C8 | 0.43 | 76.89 | 0.49 | 71.55 |
| 72F8 | 0.45 | 77.34 | 0.52 | 72.79 |

A MET-Fc chimeric protein (either human or mouse) was immobilized in solid phase and exposed to a fixed concentration of biotinylated HGF (either human or mouse), in the presence of increasing concentrations of antibodies. HGF binding to MET was revealed using HRP-conjugated streptavidin. Antibody-HGF competition is expressed as IC₅₀ (the concentration that achieves 50% competition) and I_{MAX} (the maximum % competition reached at saturation).

As a general rule, SEMA binders displaced HGF more effectively than PSI binders. In particular, those antibodies that recognize an epitope within blades 4 and 5 of the SEMA β-propeller were the most potent HGF competitors (71D6, 71C3, 71D4, 71A3, 71G2). This observation is consistent with the notion that SEMA blade 5 contains the high affinity binding site for the α-chain of HGF (Merchant et al., Proc Natl Acad Sci USA 110, E2987-2996, 2013). The PSI domain has not been shown to participate directly with HGF, but it has been suggested to function as a ‘hinge’ regulating the accommodation of HGF between the SEMA domain and the IPT region (Basilico et al., J Clin Invest. 124, 3172-3186, 2014). It is therefore likely that mAbs binding to PSI (76H10, 71G3, 76G7, 71G12) hamper HGF binding to MET by interfering with this process or by steric hindrance, and not by direct competition with the ligand. Finally, blades 1-3 of the SEMA β-propeller have been shown to be responsible for low-affinity binding of the β-chain of HGF, which plays a central role in MET activation but only partially contributes to the HGF-MET binding strength (Stamos et al., EMBO J. 23, 2325-2335, 2004). This could explain why mAbs binding to that region of MET (74C8, 72F8) are partial competitors of HGF.

### Example 6: MET Activation Assays

Due to their bivalent nature, immunoglobulins directed against receptor tyrosine kinases may display receptor agonistic activity, mimicking the effect of natural ligands. To investigate along this line, we tested the ability of human/mouse equivalent anti-MET antibodies to promote MET auto-phosphorylation in a receptor activation assay. A549 human lung carcinoma cells and MLP29 mouse liver precursor cells were deprived of serum growth factors for 48 hours and then stimulated with increasing concentrations (0-5 nM) of antibodies or recombinant HGF (A549 cells, recombinant human HGF, R&D Systems; MLP29 cells, recombinant mouse HGF, R&D Systems). After 15 minutes of stimulation, cells were washed twice with ice-cold phosphate buffered saline (PBS) and then lysed as described (Longati et al., Oncogene 9, 49-57, 1994). Protein lysates were resolved by electrophoresis and then analysed by Western blotting using antibodies specific for the phosphorylated form of MET (tyrosines 1234-1235), regardless of whether human or mouse (Cell Signaling Technology). The same lysates were also analysed by Western blotting using anti-total human MET antibodies (Invitrogen) or anti-total mouse MET antibodies (R&D Systems). This analysis revealed that all human/mouse equivalent antibodies display MET agonistic activity. Some antibodies promoted MET auto-phosphorylation to an extent comparable to that of HGF (71G3, 71D6, 71C3, 71D4, 71A3, 71G2, 74C8). Some others (76H10, 76G7, 71G12, 72F8) were less potent, and this was particularly evident at the lower antibody concentrations. No clear correlation between MET activation activity and HGF-competition activity was observed.

To obtain more quantitative data, the agonistic activity of antibodies was also characterized by phospho-MET ELISA. To this end, A549 and MLP29 cells were serum-starved as above and then stimulated with increasing concentrations (0-25 nM) of mAbs. Recombinant human (A549) or mouse (MLP29) HGF was used as control. Cells were lysed and phospho-MET levels were determined by ELISA as described (Basilico et al., J Clin Invest. 124, 3172-3186, 2014). Briefly, 96 well-plates were coated with mouse anti-human MET antibodies or rat anti-mouse MET antibodies (both from R&D Systems) and then incubated with cell lysates. After washing, captured proteins were incubated with biotin-conjugated anti-phospho-tyrosine antibodies (Thermo Fisher), and binding was revealed using HRP-conjugated streptavidin (Sigma-Aldrich).

The results of this analysis are consistent with the data obtained by Western blotting. As shown in Table 14, 71G3, 71D6, 71C3, 71D4, 71A3, 71G2 and 74C8 potently activated MET, while 76H10, 76G7, 71G12 and 72F8 caused a less pronounced effect. In any case, all antibodies displayed a comparable effect in human and in mouse cells.

TABLE 14
Agonistic activity of human/mouse equivalent
anti-MET antibodies in human and
mouse cells as measured by ELISA.

| | A549 cells | | MLP29 cells | |
|---|---|---|---|---|
| | EC₅₀ | E_{MAX} | EC₅₀ | E_{MAX} |
| mAb | (nM) | (%) | (nM) | (%) |
| 76H10 | 1.77 | 61.23 | 2.91 | 64.10 |
| 71G3 | 0.41 | 95.72 | 0.37 | 97.81 |
| 71D6 | 0.32 | 101.57 | 0.21 | 114.56 |
| 71C3 | 0.35 | 86.19 | 0.33 | 98.85 |
| 71D4 | 0.59 | 84.63 | 0.51 | 95.34 |
| 71A3 | 0.31 | 86.56 | 0.26 | 95.95 |
| 71G2 | 0.37 | 101.35 | 0.25 | 109.87 |
| 76G7 | 1.86 | 62.34 | 1.19 | 71.45 |
| 71G12 | 2.48 | 70.61 | 2.01 | 75.39 |
| 74C8 | 0.52 | 87.63 | 0.41 | 102.15 |
| 72F8 | 1.51 | 69.74 | 0.79 | 66.82 |
| HGF | 0.19 | 100.00 | 0.23 | 100.00 |

A549 human lung carcinoma cells and MLP29 mouse liver precursor cells were serum-starved and then stimulated with increasing concentrations of mAbs. Recombinant human HGF (hHGF; A549) or mouse HGF (mHGF; MLP29) was used as control. Cell lysates were analysed by ELISA using anti-total MET antibodies for capture and anti-phospho-tyrosine antibodies for revealing. Agonistic activity is expressed as EC₅₀ (nM) and E_{MAX} (% HGF activity).

### Example 7: Scatter Assay

To evaluate whether the agonistic activity of human/mouse equivalent anti-MET antibodies could translate into biological activity, we performed scatter assays with both human and mouse epithelial cells. To this end, HPAF-II human pancreatic adenocarcinoma cells (American Type Culture Collection) and MLP29 mouse liver precursor cells were stimulated with increasing concentrations of recombinant HGF (human or mouse; both from R&D Systems) and cell scattering was determined 24 hours later by microscopy as described previously (Basilico et al., J Clin Invest. 124, 3172-3186, 2014). This preliminary analysis revealed that HGF-induced cell scattering is linear until it reaches saturation at approximately 0.1 nM in both cell lines. Based on these HGF standard curves, we elaborated a scoring system ranging from 0 (total absence of cell scattering in the absence of HGF) to 4 (maximal cell scattering in the presence of 0.1 nM HGF). HPAF-II and MLP29 cells were stimulated with increasing concentrations of human/mouse equivalent anti-MET antibodies, and cell scattering was determined 24 hours later using the scoring system described above. As shown in Table 15, this analysis revealed that all mAbs tested promoted cell scattering in both the human and the mouse cell systems, with substantially overlapping results on both species. 71D6 and 71G2 displayed the very same activity as HGF; 71G3 and 71A3 were just slightly less potent than HGF; 71C3 and 74C8 required a substantially higher concentration in order to match the activity of HGF; 71D4, 76G7, 71G12 and 72F8 did not reach saturation in this assay.

TABLE 15
Biological activity of human/mouse equivalent anti-MET antibodies
as measured in a cell-based scatter assay. HPAF-II human pancreatic
adenocarcinoma cells and MLP29 mouse liver precursor cells were
stimulated with increasing concentrations of human/mouse equivalent
anti-MET antibodies, and cell scattering was determined 24 hours
later using the scoring system described in the text (0, absence
of cell scattering; 4, maximal cell scattering).

| | mAb concentration (nM) | | | | | | | | |
|---|---|---|---|---|---|---|---|---|---|
| mAb | 9.000 | 3.000 | 1.000 | 0.333 | 0.111 | 0.037 | 0.012 | 0.004 | 0.001 |

HPAF-II human pancreatic adenocarcinoma cells

| | | | | | | | | | |
|---|---|---|---|---|---|---|---|---|---|
| 76H10 | 3 | 2 | 1 | 0 | 0 | 0 | 0 | 0 | 0 |
| 71G3 | 4 | 4 | 4 | 4 | 3 | 2 | 1 | 0 | 0 |
| 71D6 | 4 | 4 | 4 | 4 | 4 | 3 | 2 | 1 | 0 |
| 71C3 | 4 | 4 | 3 | 2 | 1 | 0 | 0 | 0 | 0 |
| 71D4 | 2 | 2 | 1 | 0 | 0 | 0 | 0 | 0 | 0 |
| 71A3 | 4 | 4 | 4 | 4 | 3 | 3 | 2 | 0 | 0 |
| 71G2 | 4 | 4 | 4 | 4 | 4 | 3 | 2 | 1 | 0 |
| 76G7 | 3 | 2 | 1 | 0 | 0 | 0 | 0 | 0 | 0 |
| 71G12 | 3 | 2 | 2 | 1 | 0 | 0 | 0 | 0 | 0 |
| 74C8 | 4 | 4 | 3 | 3 | 2 | 1 | 0 | 0 | 0 |
| 72F8 | 3 | 2 | 1 | 0 | 0 | 0 | 0 | 0 | 0 |
| hHGF | 4 | 4 | 4 | 4 | 4 | 3 | 2 | 1 | 0 |
| IgG1 | 0 | 0 | 0 | 0 | 0 | 0 | 0 | 0 | 0 |

MLP29 mouse liver precursor cells

| | | | | | | | | | |
|---|---|---|---|---|---|---|---|---|---|
| 76H10 | 3 | 2 | 1 | 0 | 0 | 0 | 0 | 0 | 0 |
| 71G3 | 4 | 4 | 4 | 4 | 2 | 1 | 0 | 0 | 0 |
| 71D6 | 4 | 4 | 4 | 4 | 4 | 3 | 2 | 1 | 0 |
| 71C3 | 4 | 4 | 3 | 2 | 1 | 0 | 0 | 0 | 0 |
| 71D4 | 2 | 2 | 1 | 0 | 0 | 0 | 0 | 0 | 0 |
| 71A3 | 4 | 4 | 4 | 4 | 3 | 3 | 2 | 0 | 0 |
| 71G2 | 4 | 4 | 4 | 4 | 4 | 2 | 1 | 0 | 0 |
| 76G7 | 3 | 2 | 1 | 0 | 0 | 0 | 0 | 0 | 0 |
| 71G12 | 3 | 2 | 2 | 1 | 0 | 0 | 0 | 0 | 0 |
| 74C8 | 4 | 4 | 3 | 3 | 2 | 1 | 0 | 0 | 0 |
| 72F8 | 3 | 2 | 1 | 0 | 0 | 0 | 0 | 0 | 0 |
| mHGF | 4 | 4 | 4 | 4 | 4 | 3 | 2 | 1 | 0 |
| IgG1 | 0 | 0 | 0 | 0 | 0 | 0 | 0 | 0 | 0 |

### Example 8: Protection Against Drug-Induced Apoptosis

Several lines of experimental evidence indicate that HGF display a potent anti-apoptotic effect on MET-expressing cells (reviewed by Nakamura et al., J Gastroenterol Hepatol. 26 Suppl 1, 188-202, 2011). To test the potential anti-apoptotic activity of human/mouse equivalent anti-MET antibodies, we performed cell-based drug-induced survival assays. MCF10A human mammary epithelial cells (American Type Culture Collection) and MLP29 mouse liver precursor cells were incubated with increasing concentrations of staurosporine (Sigma Aldrich). After 48 hours, cell viability was determined by measuring total ATP concentration using the Cell Titer Glo kit (Promega) with a Victor X4 multilabel plate reader (Perkin Elmer). This preliminary analysis revealed that the drug concentration that induced about 50% cell death is 60 nM for MCF10A cells and 100 nM for MLP29 cells. Next, we incubated MCF10A cells and MLP29 cells with the above determined drug concentrations in the presence of increasing concentrations (0-32 nM) of anti-MET mAbs or recombinant HGF (human or mouse; both from R&D Systems). Cell viability was determined 48 hours later as described above. The results of this analysis, presented in Table 16, suggest that human/mouse equivalent antibodies protected human and mouse cells against staurosporine-induced cell death to a comparable extent. While some mAbs displayed a protective activity similar or superior to that of HGF (71G3, 71D6, 71G2), other molecules displayed only partial protection (76H10, 71C3, 71D4, 71A3, 76G7, 71G12, 74C8, 72F8), either in the human or in the mouse cell system.

TABLE 16
Biological activity of human/mouse
equivalent anti-MET antibodies as measured by
a cell-based drug-induced apoptosis assay.

| | MCF10A cells | | MLP29 cells | |
|---|---|---|---|---|
| mAb | EC₅₀ (nM) | E_{MAX} (%) | EC₅₀ (nM) | E_{MAX} (%) |
| 76H10 | >32.00 | 22.75 | >32.00 | 27.21 |
| 71G3 | 5.04 | 65.23 | 4.85 | 62.28 |
| 71D6 | 1.48 | 66.81 | 0.95 | 68.33 |
| 71C3 | 31.87 | 50.16 | 31.03 | 51.32 |
| 71D4 | 30.16 | 51.71 | 29.84 | 52.13 |
| 71A3 | <0.50 | 71.70 | <0.50 | 70.54 |
| 71G2 | 1.06 | 64.85 | 1.99 | 58.29 |
| 76G7 | 25.41 | 51.93 | 30.08 | 50.16 |
| 71G12 | >32.00 | 39.35 | >32.00 | 39.73 |
| 74C8 | >32.00 | 41.74 | >32.00 | 37.52 |
| 72F8 | >32.00 | 35.79 | >32.00 | 43.81 |
| HGF | 4.57 | 59.28 | 5.35 | 58.65 |

MCF10A human mammary epithelial cells and MLP29 mouse liver precursor cells were incubated with a fixed concentration of staurosporine in the the presence of increasing concentrations of anti-MET mAbs or recombinant HGF (human or mouse), and total ATP content was determined 48 hours later. Cell viability was calculated as % total ATP content relative to cells treated with neither staurosporine nor antibodies, and is expressed as EC₅₀ and E_{MAX}.

### Example 9: Branching Morphogenesis Assay

HGF is a pleiotropic cytokine which promotes the harmonic regulation of independent biological activities, including cell proliferation, motility, invasion, differentiation and survival. The cell-based assay that better recapitulates all of these activities is the branching morphogenesis assay, which replicates the formation of tubular organs and glands during embryogenesis (reviewed by Rosário and Birchmeier, Trends Cell Biol. 13, 328-335, 2003). In this assay, a spheroid of epithelial cells is seeded inside a 3D collagen matrix and is stimulated by HGF to sprout tubules which eventually form branched structures. These branched tubules resemble the hollow structures of epithelial glands, e.g. the mammary gland, in that they display a lumen surrounded by polarized cells. This assay is the most complete HGF assay that can be run in vitro.

In order to test whether human/mouse equivalent anti-MET antibodies displayed agonistic activity in this assay, we seeded LOC human kidney epithelial cells (Michieli et al. Nat Biotechnol. 20, 488-495, 2002) and MLP29 mouse liver precursor cells in a collagen layer as described (Hultberg et al., Cancer Res. 75, 3373-3383, 2015), and then exposed them to increasing concentrations of mAbs or recombinant HGF (human or mouse, both from R&D Systems). Branching morphogenesis was followed over time by microscopy, and colonies were photographed after 5 days. Quantification of branching morphogenesis activity was obtained by counting the number of branches for each spheroid. As shown in Table 17, all antibodies tested induced dose-dependent formation of branched tubules. However, consistent with the data obtained in MET auto-phosphorylation assays and cell scattering assays, 71D6, 71A3 and 71G2 displayed the most potent agonistic activity, similar or superior to that of recombinant HGF.

TABLE 17
Branching morphogenesis assay.

| mAb | 0 nM | 0.5 nM | 2.5 nM | 12.5 nM |
|---|---|---|---|---|
| LOC cells | | | | |
| 76H10 | 3.3 ± 1.5 | 7.3 ± 0.6 | 11.7 ± 1.5 | 16.7 ± 1.5 |
| 71G3 | 3.0 ± 1.0 | 13.7 ± 1.5 | 19.0 ± 2.6 | 22.3 ± 2.1 |
| 71D6 | 3.0 ± 1.0 | 29.0 ± 2.0 | 29.0 ± 2.6 | 32.7 ± 1.5 |
| 71C3 | 3.3 ± 0.6 | 8.7 ± 1.5 | 12.7 ± 2.1 | 15.7 ± 2.1 |
| 71D4 | 3.0 ± 1.0 | 9.0 ± 2.6 | 15.7 ± 1.2 | 18.7 ± 1.5 |
| 71A3 | 3.0 ± 1.7 | 24.0 ± 4.6 | 30.3 ± 3.2 | 31.3 ± 1.5 |
| 71G2 | 3.7 ± 1.5 | 25.3 ± 2.1 | 29.3 ± 3.5 | 31.7 ± 3.5 |
| 76G7 | 2.7 ± 0.6 | 6.7 ± 0.6 | 13.3 ± 4.2 | 16.3 ± 5.7 |
| 71G12 | 3.3 ± 0.6 | 7.0 ± 2.6 | 15.3 ± 5.5 | 16.0 ± 4.6 |
| 74C8 | 3.0 ± 1.0 | 10.3 ± 4.2 | 17.0 ± 4.6 | 18.7 ± 4.9 |
| 72F8 | 3.3 ± 1.5 | 9.0 ± 3.5 | 12.3 ± 2.1 | 16.0 ± 3.0 |
| hHGF | 3.0 ± 1.0 | 18.0 ± 2 | 27.7 ± 2.5 | 20.3 ± 2.1 |
| MLP29 cells | | | | |
| 76H10 | 0.3 ± 0.6 | 10.7 ± 4.0 | 14.3 ± 3.2 | 24.7 ± 6.0 |
| 71G3 | 0.3 ± 0.6 | 24.7 ± 4.5 | 34.3 ± 5.5 | 29.3 ± 8.0 |
| 71D6 | 1.3 ± 1.2 | 32.7 ± 3.5 | 39.0 ± 7.5 | 41.3 ± 8.0 |
| 71C3 | 0.3 ± 0.6 | 11.7 ± 3.5 | 15.7 ± 6.5 | 24.7 ± 6.5 |
| 71D4 | 0.7 ± 1.2 | 16.0 ± 2.6 | 14.7 ± 4.5 | 21.7 ± 5.5 |
| 71A3 | 0.7 ± 0.6 | 30.3 ± 2.1 | 42.0 ± 6.2 | 42.7 ± 8.0 |
| 71G2 | 1.0 ± 1.0 | 34.0 ± 2.6 | 46.3 ± 4.7 | 45.0 ± 7.0 |
| 76G7 | 0.3 ± 0.6 | 14.7 ± 2.1 | 18.7 ± 4.5 | 24.7 ± 6.5 |
| 71G12 | 1.0 ± 1.0 | 14.0 ± 2.6 | 14.7 ± 5.5 | 22.7 ± 6.0 |
| 74C8 | 0.7 ± 0.6 | 17.3 ± 2.5 | 15.3 ± 6.0 | 22.3 ± 9.0 |
| 72F8 | 1.0 ± 1.0 | 12.7 ± 3.1 | 11.7 ± 3.5 | 18.7 ± 2.5 |
| mHGF | 0.7 ± 1.2 | 32.3 ± 4.0 | 43.7 ± 4.2 | 36.0 ± 7.2 |

Cell spheroids preparations of LOC human kidney epithelial cells or MLP29 mouse liver precursor cells were seeded in a collagen layer and then incubated with increasing concentrations (0, 0.5, 2.5 and 12.5 nM) of mAbs or recombinant HGF (LOC, human HGF; MLP29, mouse HGF). Branching morphogenesis was followed over time by microscopy, and colonies were photographed after 5 days. Branching was quantified by counting the number of branches for each spheroid (primary branches plus secondary branches).

### Example 10: Human-Mouse Equivalent Agonistic Anti-MET Antibodies

TABLE 18
Major biochemical and biological characteristics of human/mouse equivalent
anti-MET antibodies. This table summarizes the ability of each mAb to bind
to purified MET ECD (ELISA), to recognize native MET on MET-expressing cells
(FACS), to compete with HGF for MET binding (HGF competition), to activate
MET in receptor auto-phosphorylation assays (MET activation), to promote cell
scattering (Scatter assay), to protect cells against drug-induced apoptosis
(Survival assay), and to promote branching morphogenesis of epithelial cell
spheroids (Branching morphogenesis). For each assay, the score is based on
the relative activity of any given mAb with respect to the other antibodies
(+, lower 50%; ++, upper 50%). Since all antibodies displayed similar
activities in human and mouse systems, only one score per assay is shown.

| | | Biochemical activity | | | Biological activity | | | |
|---|---|---|---|---|---|---|---|---|
| | | | | HGF | MET | Scatt. | Surv. | Bran. |
| mAb | VH | ELISA | FACS | comp. | activ. | Assay | Assay | morph. |
| 76H10 | 1 | ++ | + | + | + | + | + | + |
| 71G3 | 2 | ++ | ++ | + | ++ | ++ | ++ | ++ |
| 71D6 | 3a | ++ | ++ | ++ | ++ | ++ | ++ | ++ |
| 71C3 | 3b | + | ++ | ++ | ++ | ++ | + | + |
| 71D4 | 3c | + | ++ | ++ | ++ | + | + | + |
| 71A3 | 4 | + | ++ | ++ | ++ | ++ | ++ | ++ |
| 71G2 | 4 | ++ | ++ | ++ | ++ | ++ | ++ | ++ |
| 76G7 | 5 | ++ | + | + | + | + | + | + |
| 71G12 | 6 | ++ | + | + | + | + | + | + |
| 74C8 | 9 | + | ++ | + | + | ++ | + | + |
| 72F8 | 10 | ++ | ++ | + | ++ | + | + | + |

### Example 11: Constant Region Swapping does not Alter the Biochemical and Biological Features of Human/Mouse Equivalent Antibodies

We sought to determine whether swapping of the human heavy chain and light chain constant regions with the corresponding mouse constant regions affected the major biochemical and biological activities of a representative panel of antibodies. To this end, we selected 3 representative molecules from the panel of human/mouse equivalent antibodies (71 G3, partial competitor of HGF and partial agonist in biological assays; 71D6 and 71 G2, full competitors of HGF and full agonists in biological assays). The VH and VL regions of 71G3, 71D6 and 71G2 were mounted onto mouse IgG1/λ antibody frames. The sequences of all mouse immunoglobulin variants are available in public databases such as the ImMunoGeneTics information system (www.imgt.org). Fusion with the desired variable regions can be achieved by standard genetic engineering procedures. The full amino acid sequences of the heavy chain and light chains of the generated llama-mouse chimeric antibodies are shown in Table 19.

TABLE 19
Full heavy chain and light chain amino acid sequences of llama-mouse
chimeric mAbs binding to both human and mouse MET.

| | | SEQ ID | | SEQ ID |
|---|---|---|---|---|
| Clone | Heavy chain (VH-CH1-CH2-CH3) | NO. | Light chain (VL-CL) | NO. |
| 71G3 | QVQLVESGGGLVQPGGSLRVSCAA | 211 | QAVVTQEPSLSVSPGGT | 212 |
| | SGFTFSTYYMSWVRQAPGKGLEW | | VTLTCGLSSGSVTTSNY | |
| | VSDIRTDGGTYYADSVKGRFTMSR | | PGWFQQTPGQAPRTLIY | |
| | DNAKNTLYLQMNSLKPEDTALYYCA | | NTNSRHSGVPSRFSGSI | |
| | RTRIFPSGYDYWGQGTQVTVSSAK | | SGNKAALTIMGAQPEDE | |
| | TTPPSVYPLAPGSAAQTNSMVTLGC | | ADYYCSLYPGSTTVFGG | |
| | LVKGYFPEPVTVTWNSGSLSSGVH | | GTHLTVLGQPKSSPSVT | |
| | TFPAVLQSDLYTLSSSVTVPSSPRP | | LFPPSSEELETNKATLVC | |
| | SETVTCNVAHPASSTKVDKKIVPRD | | TITDFYPGVVTVDWKVD | |
| | CGCKPCICTVPEVSSVFIFPPKPKDV | | GTPVTQGMetETTQPSK | |
| | LTITLTPKVTCVVVDISKDDPEVQFS | | QSNNKYMetASSYLTLTA | |
| | WFVDDVEVHTAQTQPREEQFNSTF | | RAWERHSSYSCQVTHE | |
| | RSVSELPIMHQDWLNGKEFKCRVN | | GHTVEKSLSRADCS | |
| | SAAFPAPIEKTISKTKGRPKAPQVYT | | | |
| | IPPPKEQMAKDKVSLTCMITDFFPE | | | |
| | DITVEWQWNGQPAENYKNTQPIMN | | | |
| | TNGSYFVYSKLNVQKSNWEAGNTF | | | |
| | TCSVLHEGLHNHHTEKSLSHSPGK | | | |
| 71D6 | ELQLVESGGGLVQPGGSLRLSCAA | 213 | QPVLNQPSALSVTLGQT | 214 |
| | SGFTFSSYGMSVVVRQAPGKGLEW | | AKITCQGGSLGARYAHW | |
| | VSAINSYGGSTSYADSVKGRFTISR | | YQQKPGQAPVLVIYDDD | |
| | DNAKNTLYLQMNSLKPEDTAVYYCA | | SRPSGIPERFSGSSSGG | |
| | KEVRADLSRYNDYESYDYWGQGT | | TATLTISGAQAEDEGDY | |
| | QVTVSSAKTTPPSVYPLAPGSAAQT | | YCQSADSSGSVFGGGT | |
| | NSMVTLGCLVKGYFPEPVTVTWNS | | HLTVLGQPKSSPSVTLF | |
| | GSLSSGVHTFPAVLQSDLYTLSSSV | | PPSSEELETNKATLVCTI | |
| | TVPSSPRPSETVTCNVAHPASSTKV | | TDFYPG\NTVDWKVDG | |
| | DKKIVPRDCGCKPCICTVPEVSSVFI | | TPVTQGMetETTQPSKQ | |
| | FPPKPKDVLTITLTPKVTCVVVDISK | | SNNKYMetASSYLTLTAR | |
| | DDPEVQFSWFVDDVEVHTAQTQPR | | AWERHSSYSCQVTHEG | |
| | EEQFNSTFRSVSELPIMHQDWLNG | | HTVEKSLSRADCS | |
| | KEFKCRVNSAAFPAPIEKTISKTKGR | | | |
| | PKAPQVYTIPPPKEQMAKDKVSLTC | | | |
| | MITDFFPEDITVEWQWNGQPAENY | | | |
| | KNTQPIMNTNGSYFVYSKLNVQKSN | | | |
| | WEAGNTFTCSVLHEGLHNHHTEKS | | | |
| | LSHSPGK | | | |
| 71G2 | EVQLQESGGGLVQPGGSLRLSCAA | 215 | SSALTQPSALSVSLGQT | 216 |
| | SGFTFSIYDMSWVRQAPGKGLEWV | | ARITCQGGSLGSSYAHW | |
| | STINSDGSSTSYVDSVKGRFTISRD | | YQQKPGQAPVLVIYGDD | |
| | NAKNTLYLQMNSLKPEDTAVYYCAK | | SRPSGIPERFSGSSSGG | |
| | VYGSTWDVGPMGYGMDYWGKGTL | | TATLTISGAQAEDEDDYY | |
| | VTVSSAKTTPPSVYPLAPGSAAQTN | | CQSTDSSGNTVFGGGT | |
| | SMVTLGCLVKGYFPEPVTVTWNSG | | RLTVLGQPKSSPSVTLF | |
| | SLSSGVHTFPAVLQSDLYTLSSSVT | | PPSSEELETNKATLVCTI | |
| | VPSSPRPSETVTCNVAHPASSTKVD | | TDFYPG\NTVDWKVDG | |
| | KKIVPRDCGCKPCICTVPEVSSVFIF | | TPVTQGMetETTQPSKQ | |
| | PPKPKDVLTITLTPKVTCVVVDISKD | | SNNKYMetASSYLTLTAR | |
| | DPEVQFSWFVDDVEVHTAQTQPRE | | AWERHSSYSCQVTHEG | |
| | EQFNSTFRSVSELPIMHQDWLNGK | | HTVEKSLSRADCS | |
| | EFKCRVNSAAFPAPIEKTISKTKGRP | | | |
| | KAPQVYTIPPPKEQMAKDKVSLTCM | | | |
| | ITDFFPEDITVEWQWNGQPAENYKN | | | |
| | TQPIMNTNGSYFVYSKLNVQKSNW | | | |
| | EAGNTFTCSVLHEGLHNHHTEKSLS | | | |
| | HSPGK | | | |

Production and purification of recombinant immunoglobulins can be obtained by transient transfection in mammalian cells and affinity chromatography, respectively, following well established protocols. Thereafter, we compared the biochemical and biological activities of 71G3, 71D6 and 71G2 in the mouse format with those of the same antibodies in the human format.

We evaluated the ability of the antibodies to bind to purified human or mouse MET ECD by ELISA, to recognize native MET on human or mouse cells by FACS, to induce scattering of human and mouse epithelial cells, and to promote branching morphogenesis in collagen. The results of this analysis, summarized in Table 20, reveal that swapping the human with the mouse constant regions does not substantially affect any of the properties analysed.

TABLE 20
Constant region swapping does not alter the biochemical and biological
features of human/mouse equivalent antibodies. Three representative
agonistic antibodies (71G3, 71D6 and 71G2) in either mouse or human
format were subjected to several in vitro assays aimed at characterizing
their major biochemical and biological properties.

| | 71G3 | | 71D6 | | 71G2 | |
|---|---|---|---|---|---|---|
| Assay | Human | Mouse | Human | Mouse | Human | Mouse |
| (measure unit) | IgG1/λ | IgG1/λ | IgG1/λ | IgG1/λ | IgG1/λ | IgG1/λ |
| hMET ELISA | 0.061 ± | 0.067 ± | 0.032 ± | 0.038 ± | 0.109 ± | 0.113 ± |
| (EC₅₀, nM) | 0.024 | 0.026 | 0.015 | 0.014 | 0.038 | 0.023 |
| mMET ELISA | 0.059 ± | 0.062 ± | 0.036 ± | 0.036 ± | 0.101 ± | 0.109 ± |
| (EC₅₀, nM) | 0.035 | 0.028 | 0.022 | 0.025 | 0.029 | 0.021 |
| A549 FACS | 110.5 ± | 115.7 ± | 115.2 ± | 121.9 ± | 137.0 ± | 141.7 ± |
| (E_{MAX}, % CTR) | 15.3 | 17.2 | 9.7 | 11.4 | 19.1 | 12.5 |
| MLP29 FACS | 112.5 ± | 109.7 ± | 120.4 ± | 118.6 ± | 130.7 ± | 127.7 ± |
| (E_{MAX}, % CTR) | 11.3 | 13.2 | 14.1 | 15.8 | 18.3 | 12.1 |
| A549 MET act. | 94.3 ± | 90.8 ± | 103.7 ± | 98.3 ± | 105.5 ± | 101.5 ± |
| (E_{MAX}, % HGF) | 9.8 | 8.9 | 7.9 | 9.5 | 9.6 | 8.2 |
| MLP29 MET act. | 96.8 ± | 91.9 ± | 110.5 ± | 103.4 ± | 109.7 ± | 102.5 ± |
| (E_{MAX}, % HGF) | 8.8 | 8.4 | 8.5 | 7.9 | 9.8 | 4.7 |
| LOC br. m. | 22.3 ± | 20.8 ± | 32.7 ± | 30.4 ± | 31.7 ± | 29.8 ± |
| (branch n.) | 2.1 | 3.5 | 1.5 | 3.7 | 3.5 | 4.1 |
| MLP29 br. m. | 29.3 ± | 30.1 ± | 41.3 ± | 39.5 ± | 45.0 ± | 41.2 ± |
| (branch n.) | 8.0 | 7.3 | 8.0 | 6.1 | 7.0 | 6.3 |

### Example 12: Comparison with Prior Art Antibodies: Human-Mouse Cross-Reactivity

As discussed in detail in the Background section, a few other studies have already described agonistic anti-MET antibodies that mimic HGF activity, at least partially. At the time of writing, these include: (i) the 3D6 mouse anti-human MET antibody (U.S. Pat. No. 6,099,841); (ii) the 5DS mouse anti-human MET antibody (U.S. Pat. No. 5,686,292); (iii) the NO-23 mouse anti-human MET antibody (U.S. Pat. No. 7,556,804 B2); (iv) the B7 human naïve anti-human MET antibody (U.S. Patent Application No. 2014/0193431 A1); (v) the DO-24 mouse anti-human MET antibody (Prat et al., Mol Cell Biol. 11, 5954-5962, 1991; Prat et al., J Cell Sci. 111, 237-247, 1998); and (vi) the DN-30 mouse anti-human MET antibody (Prat et al., Mol Cell Biol. 11, 5954-5962, 1991; Prat et al., J Cell Sci. 111, 237-247, 1998).

We obtained all prior art agonistic anti-MET antibodies as follows. The 3D6 hybridoma was purchased from the American Type Culture Collection (Cat. No. ATCC-HB-12093). The 3D6 antibody was purified from the hybridoma conditioned medium by standard affinity chromatography protocols.

The cDNA encoding the variable regions of the 5DS antibody, the bivalent progenitor of the antagonistic anti-MET antibody Onartuzumab (Merchant et al., Proc Natl Acad Sci USA 110, E2987-2996, 2013), were synthesized based on the VH and VL sequences published in U.S. Pat. No. 7,476,724 B2. The obtained DNA fragments were fused with mouse constant IgG1/λ domains and produced as bivalent monoclonal antibodies by standard protein engineering protocols.

The NO-23 antibody was obtained from Prof. Maria Prat, University of Novara, Italy (inventor of NO-23; U.S. Pat. No. 7,556,804 B2). The NO-23 antibody can also be obtained by requesting the corresponding hybridoma to the international depositary authority Interlab Cell Line Collection (ICLC) at the Advanced Biotechnology Center (ABC) in Genova, Italy (Clone No. ICLC 03001).

The cDNA encoding the variable regions of the B7 antibody were synthesized based on the VH and VL sequences published in US Patent Application No. 2014/0193431 A1. The obtained DNA fragments were fused with mouse constant IgG1/λ domains and produced as bivalent monoclonal antibodies as described above.

The DO-24 and DN-30 antibodies were obtained from Prof. Maria Prat, University of Novara, Italy (who first identified and characterized DO-24 and DN-30; Prat at al., Mol Cell Biol. 11, 5954-5962, 1991; Prat et al., J Cell Sci. 111, 237-247, 1998). The DO-24 antibody, now discontinued, has been commercially available for years from Upstate Biotechnology. The DN-30 antibody can also be obtained by requesting the corresponding hybridoma to the international depositary authority Interlab Cell Line Collection (ICLC) at the Advanced Biotechnology Center (ABC) in Genoa, Italy (Clone No. ICLC PD 05006).

Because the vast majority of animal models of human diseases employ the mouse as a host, cross-reactivity with the mouse antigen is an essential pre-requisite for an antibody the biological activity of which needs to be validated in pre-clinical systems. Since all antibodies of the prior art were generated in a mouse (except for B7 that was identified using a human naïve phage library), it is unlikely that these molecules display cross-reactivity with mouse MET. Even if a minor cross-reactivity with self-antigens is in principle possible, these interactions have normally a very low affinity.

As detailed in U.S. Pat. No. 6,099,841, the 3D6 antibody does not bind to mouse MET and the inventors had to use ferrets and minks to demonstrate that their antibody has in vivo activity. It is clear that these animal models do not represent ideal systems for modelling human diseases nor their use in preclinical medicine has been established. Furthermore, the inventors do not provide any quantitative data relative to the difference in antibody affinity and activity between human systems and ferret or mink systems.

The 5DS antibody and its derivatives were explicitly shown not to bind to mouse MET (Merchant et al., Proc Natl Acad Sci USA 110, E2987-2996, 2013). No information is available about its cross-reactivity with other preclinical species.

Likewise, U.S. Patent Application No. 2014/0193431 A1 provides no information relative to cross-reactivity of the B7 antibody with mouse MET or that of other species.

U.S. Pat. No. 7,556,804 B2 claims that the NO-23 antibody cross-reacts with mouse, rat and dog MET, but no quantitative experimental evidence is provided in support of this statement. The inventors use a single saturating dose of NO-23 to immuno-precipitate MET from lysates of mouse, rats, human or dog cells, and then incubate the immuno-precipitated proteins with radioactive ³²P-ATP. After radiolabeling, the incorporated ³²P-ATP is visualized by autoradiography. This method is extremely sensitive and by no mean quantitative; it is not possible to tell to what percentage of cross-reactivity the bands on the gel correspond to.

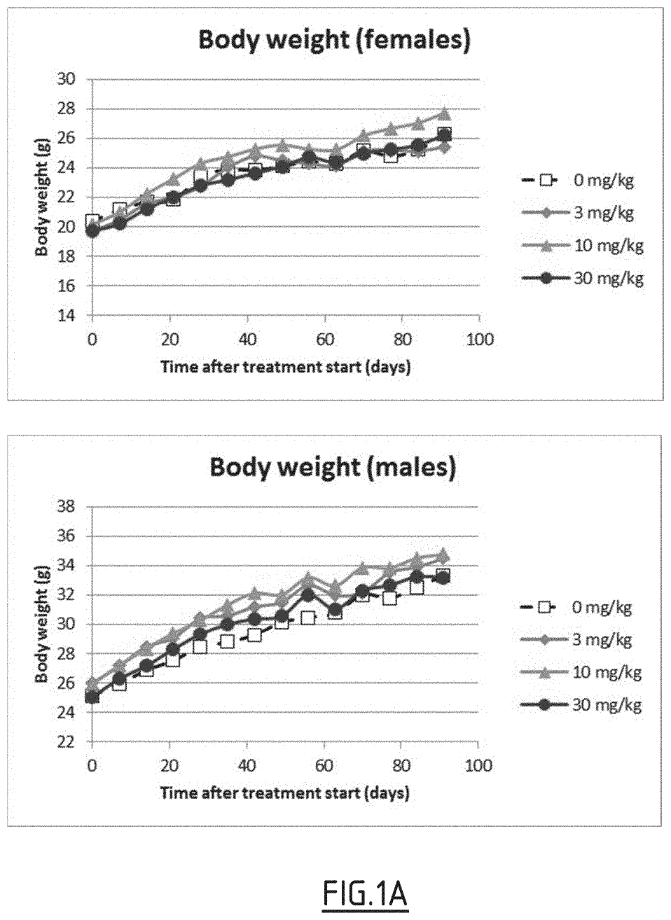

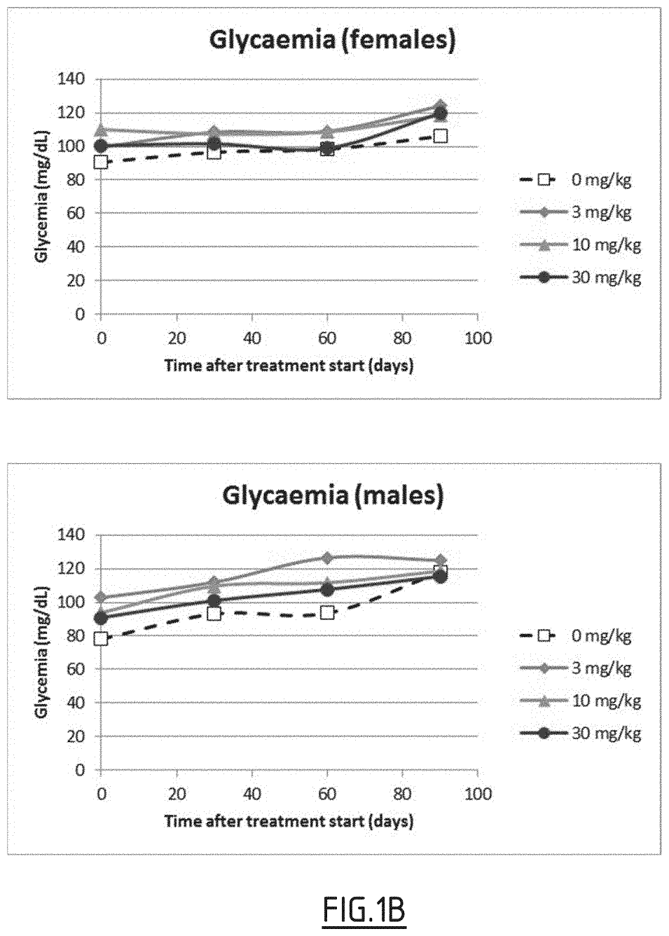

Similarly, the DO-24 antibody is suggested to cross-react with mouse MET because a DO-24-containing Matrigel pellet promotes blood vessel recruitment when implanted in the abdominal cavity of a mouse (Prat et al., J Cell Sci. 111, 237-247, 1998). However, this could also be due to increased inflammation and no direct evidence that DO-24 interacts with mouse MET is provided. In a different study, a single saturating dose of DO-24 (20 nM) is shown to cause auto-phosphorylation of MET in the rat cardiac muscle cell line H9c2 and in the mouse cardiac muscle cell line HL-5 (Pietronave et al., Am J Physiol Heart Circ Physiol. 298, H1155-65, 2010; FIG. 1). In the same experiment, a much lower dose of recombinant HGF (0.5 nM) is shown to cause MET phosphorylation to a comparable extent. As the authors themselves acknowledge in the Discussion section, these results suggest that DO-24 is dramatically less potent than HGF in these rodent cell lines. Since DO-24 is claimed by the same authors to be a full agonistic mAb that matches HGF activity in human cell models (Prat et al., J Cell Sci. 111, 237-247, 1998), then it should be concluded that DO-24 does not elicit the same efficacy or potency in human and in mouse cells. Furthermore, it should be noted that the experiments shown by Pietronave et al. are not quantitative and are not useful to extract information on the degree of cross-reactivity that occurs between DO-24 and mouse or rat MET, the measurement of which would require a head-to-head dose-response study, like the one that we did (see below). In a third work, a mixture of the DO-24 and DN-30 antibodies is used to immuno-precipitate MET from mouse mesenchymal stem cell lysates (Forte at al., Stem Cells. 24, 23-33, 2006). Both the presence of DN-30 and the assay type (immuno-precipitation from cell lysates) prevent to obtain precise information on the ability of DO-24 to interact with native mouse MET. In conclusion, no experimental evidence whatsoever exists that the DO-24 antibody elicits comparable biological responses in human and in mouse cells.

Finally, the DN-30 antibody was explicitly shown not to interact with mouse MET (Prat at al., J Cell Sci. 111, 237-247, 1998; and suppl. material of Petrelli et al., Proc Natl Acad Sci USA 103, 5090-9095, 2006).

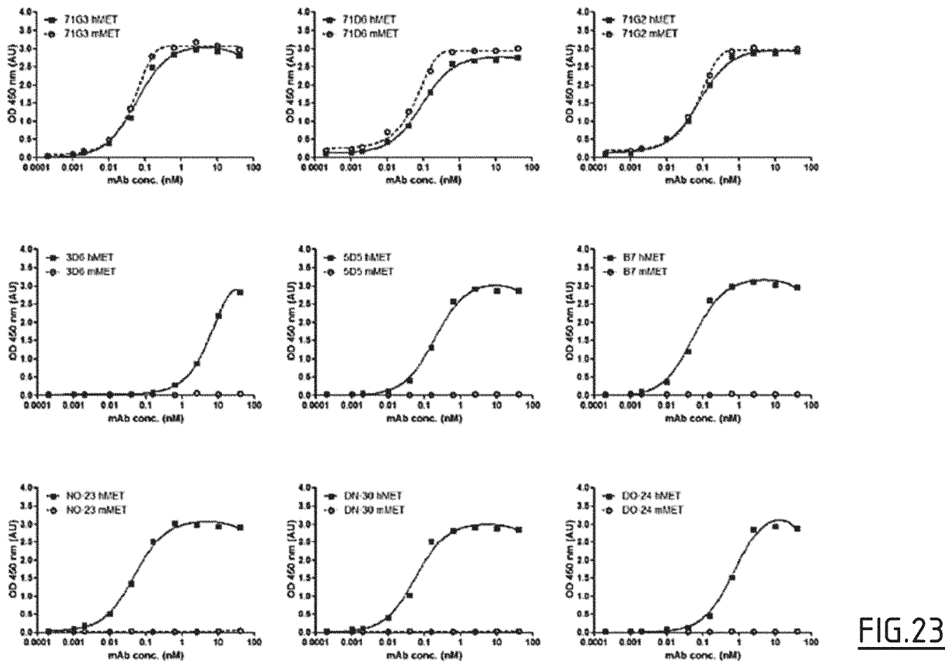

In order to directly determine whether—and to what extent—the prior art agonistic anti-MET antibodies cross-reacted with mouse MET, and to compare them to our human/mouse equivalent anti-MET antibodies, we performed an ELISA assay. Since all prior art antibodies were obtained or engineered with a mouse IgG/λ format, we employed the mouse IgG/λ version of 71G3, 71D6 and 71G2. Human or mouse MET ECD was immobilized in solid phase (100 ng/well in a 96-well plate) and exposed to increasing concentrations of antibodies (0-40 nM) in solution. Binding was revealed using HRP-conjugated anti-mouse Fc antibodies (Jackson Immuno Research Laboratories). As shown in Table 21, this analysis revealed that, while the prior art antibodies bound to human MET with a K_{D} ranging from 0.059 nM (B7) to 4.935 nM (3D6), none of them displayed any affinity for mouse MET, even at a concentration as high as 40 nM. Among the antibodies tested, only 71G3, 71D6 and 71G2 bound to both human and mouse MET, and they did so with indistinguishable affinities and capacities. The results of this analysis are shown in FIG. 23.

TABLE 21
Binding affinity and capacity of anti-MET antibodies
for human and mouse MET as determined by ELISA.

| | hMET | | mMET | |
|---|---|---|---|---|
| mAb | EC₅₀ | E_{MAX} | EC₅₀ | E_{MAX} |
| 71G3 | 0.058 | 3.107 | 0.059 | 3.065 |
| 71D6 | 0.042 | 2.688 | 0.044 | 2.941 |
| 71G2 | 0.098 | 2.857 | 0.091 | 2.963 |
| 3D6 | 4.935 | 3.208 | >40.000 | n.c. |
| 5D5 | 0.197 | 3.162 | >40.000 | n.c. |
| B7 | 0.059 | 3.272 | >40.000 | n.c. |
| NO-23 | 0.063 | 3.106 | >40.000 | n.c. |
| DO-24 | 0.761 | 3.321 | >40.000 | n.c. |
| DN-30 | 0.067 | 3.064 | >40.000 | n.c. |

Affinity is expressed as EC₅₀ (nMol/L). Capacity is expressed as E_{MAX} (optical density at 450 nm; n.c., not converged). See FIG. 23 for the entire binding profiles.

### Example 13: Comparison with Prior Art Antibodies: MET Auto-Phosphorylation

In order to compare the agonistic activity of the prior art antibodies with that of human/mouse equivalent anti-MET antibodies, we performed a MET auto-phosphorylation experiment using both human and mouse cells. A549 human lung carcinoma cells and MLP29 mouse liver precursor cells were deprived of serum growth factors for 48 hours and then stimulated with increasing concentrations of antibodies (0-25 nM). After 15 minutes of stimulation, cells were washed twice with ice-cold phosphate buffered saline (PBS) and then lysed as described (Longati et al., Oncogene 9, 49-57, 1994). Phospho-MET levels were determined by ELISA as described (Basilico et al., J Clin Invest. 124, 3172-3186, 2014) using anti-MET antibodies for capture (R&D Systems) and anti-phospho tyrosines for revealing (R&D Systems).

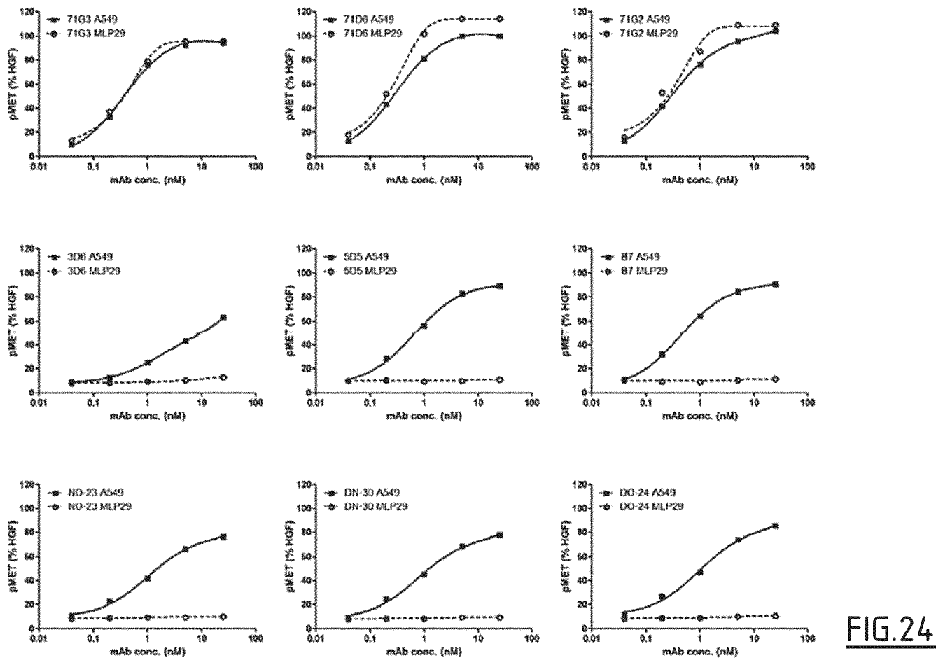

This analysis revealed two major differences between prior art antibodies and the human/mouse equivalent anti-MET antibodies described in the present document. First, consistent with the results obtained in binding experiments, only 71G3, 71D6 and 71G2 could promote MET auto-phosphorylation in both human and mouse cells. The prior art antibodies, including DO-24 and NO-23, induced MET activation in human cells only; no activity on mouse cells could be detected in the system that we analyzed. Second, all prior art antibodies invariably displayed lower agonistic activity compared to 71G3, 71D6 and 71G2. The most agonistic prior art mAbs were 5DS and B7, which displayed an activity slightly lower than 71G3, 71D6 and 71G2. The least agonistic prior art mAb was 3D6. The other molecules displayed intermediate activity. The results of this analysis are shown in FIG. 24.

### Example 14: Comparison with Prior Art Antibodies: Branching Morphogenesis

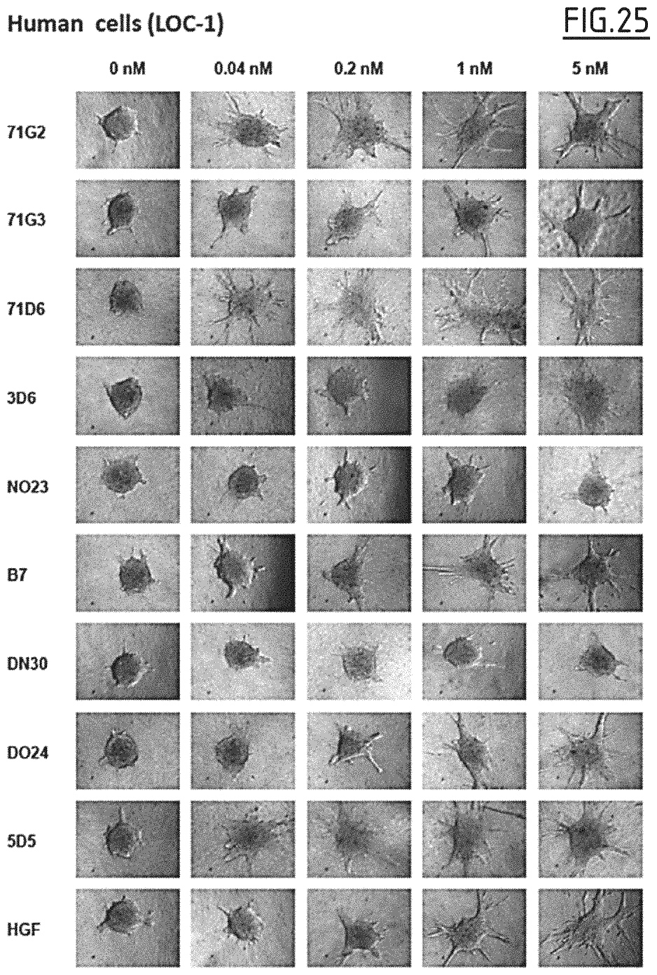

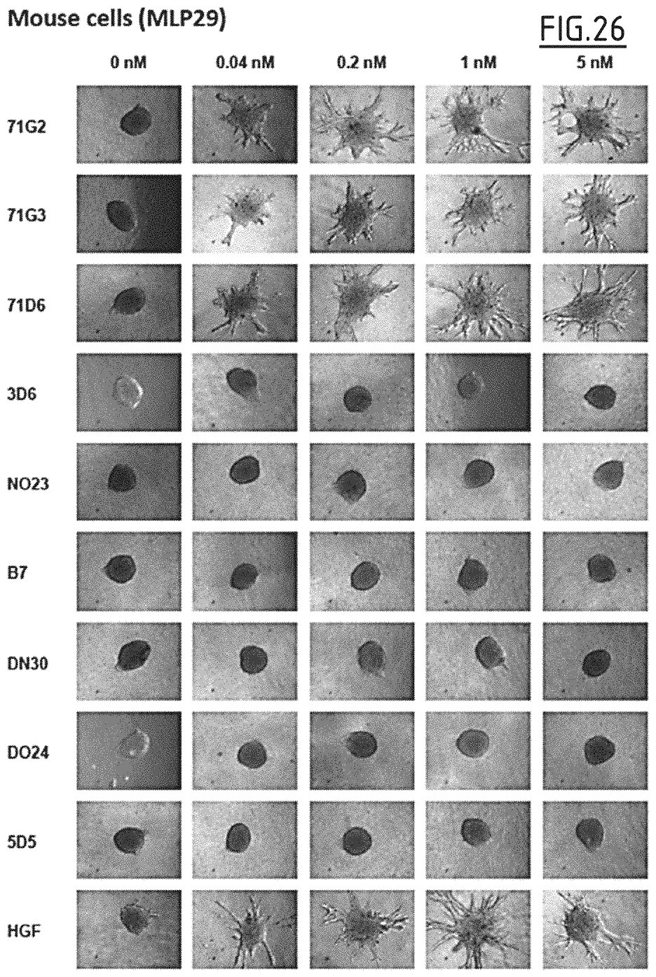

In order to compare the biological activity of prior art antibodies with that of human/mouse equivalent anti-MET antibodies, we performed a branching morphogenesis assay. This assay recapitulates all the relevant biological activities of HGF including cell proliferation, scattering, differentiation and survival. LOC human kidney epithelial cells and MLP29 mouse liver precursor cells were seeded in a collagen layer as described above and then incubated with increasing concentrations of mAbs or recombinant HGF (human or mouse, both from R&D Systems). Branching morphogenesis was followed over time by microscopy, and colonies were photographed after 5 days. Quantification of branching morphogenesis activity was achieved by counting the number of branched tubules sprouting from each spheroid and is shown in Table 22. Representative images of spheroids are shown in FIG. 25 (LOC cells) and in FIG. 26 (MLP29 cells).

TABLE 22
Branching morphogenesis assay.

| mAb | 0 nM | 0.04 nM | 0.2 nM | 1 nM | 5 nM |
|---|---|---|---|---|---|
| LOC cells | | | | | |
| 71G3 | 2.7 ± 0.6 | 9.0 ± 1.0 | 13.3 ± 1.5 | 17.7 ± 1.5 | 20.7 ± 1.2 |
| 71D6 | 2.3 ± 0.6 | 18.7 ± 3.2 | 29.3 ± 2.5 | 30.7 ± 2.1 | 30.3 ± 1.2 |
| 71G2 | 2.7 ± 1.5 | 22.3 ± 2.3 | 26.3 ± 2.1 | 30.0 ± 2.0 | 30.3 ± 3.5 |
| 3D6 | 2.3 ± 0.6 | 4.0 ± 1.0 | 7.0 ± 1.0 | 10.7 ± 1.5 | 19.3 ± 4.2 |
| 5D5 | 4.3 ± 1.5 | 15.7 ± 1.5 | 18.3 ± 1.5 | 21.3 ± 2.1 | 27.7 ± 1.5 |
| B7 | 3.3 ± 1.5 | 8.7 ± 1.5 | 13.3 ± 1.5 | 19.7 ± 1.5 | 24.0 ± 2.0 |
| NO-23 | 3.3 ± 1.2 | 6.0 ± 1.0 | 7.0 ± 1.0 | 8.7 ± 1.2 | 8.7 ± 1.5 |
| DO-24 | 3.3 ± 2.1 | 8.0 ± 1.0 | 12.0 ± 1.0 | 12.3 ± 1.2 | 17.7 ± 2.1 |
| DN-30 | 3.3 ± 0.6 | 6.3 ± 1.5 | 8.3 ± 1.5 | 9.7 ± 1.5 | 10.3 ± 1.5 |
| hHGF | 4.7 ± 1.5 | 10.7 ± 1.5 | 16.7 ± 1.5 | 28.3 ± 3.5 | 24.7 ± 7.6 |
| MLP29 cells | | | | | |
| 71G3 | 0.3 ± 0.6 | 19.3 ± 1.5 | 23.7 ± 2.1 | 32.7 ± 2.5 | 28.7 ± 1.2 |
| 71D6 | 0.7 ± 0.6 | 21.0 ± 2.0 | 32.0 ± 1.0 | 42.7 ± 5.5 | 37.0 ± 2.0 |
| 71G2 | 0.0 ± 0.0 | 15.0 ± 1.7 | 36.0 ± 4.6 | 50.7 ± 5.5 | 48.0 ± 3.6 |
| 3D6 | 0.3 ± 0.6 | 0.7 ± 0.6 | 0.7 ± 0.6 | 0.7 ± 0.6 | 0.7 ± 0.6 |
| 5D5 | 1.0 ± 1.0 | 0.7 ± 1.2 | 0.3 ± 0.6 | 1.3 ± 1.5 | 1.0 ± 1.0 |
| B7 | 0.3 ± 0.6 | 0.7 ± 0.6 | 0.3 ± 0.6 | 1.3 ± 1.5 | 0.7 ± 1.2 |
| NO-23 | 0.7 ± 1.2 | 0.3 ± 0.6 | 0.7 ± 0.6 | 1.0 ± 1.0 | 0.7 ± 0.6 |
| DO-24 | 1.0 ± 1.0 | 0.7 ± 1.2 | 0.7 ± 0.6 | 0.7 ± 1.2 | 0.7 ± 1.2 |
| DN-30 | 0.7 ± 0.6 | 0.3 ± 0.6 | 1.0 ± 1.0 | 0.7 ± 0.6 | 0.7 ± 0.6 |
| mHGF | 0.3 ± 0.6 | 26.0 ± 4.4 | 34.0 ± 5.0 | 46.0 ± 2.6 | 37.0 ± 2.0 |

Cell spheroids preparations of LOC human kidney epithelial cells or MLP29 mouse liver precursor cells were seeded in a collagen layer and then incubated with increasing concentrations (0, 0.04, 0.2, 1, and 5 nM) of mAbs or recombinant human HGF (LOC) or mouse HGF (MLP29). Branching morphogenesis was followed over time by microscopy, and colonies were photographed after 5 days. Branching was quantified by counting the number of branches for each spheroid (primary branches plus secondary branches).

The data presented lead to the following observations. In human cells, 71D6, 71G2 and 5D5 displayed an activity comparable to that of human HGF; 71 G3, 316, B7 and DO-24 behaved as partial agonists; NO-23 and DN-30 displayed very little agonistic activity. In mouse cells, only 71G3, 71D6 and 71G2 effectively induced the formation of branched tubules; all the other antibodies—consistent with their inability to bind to mouse MET in ELISA—did not induce branching morphogenesis at all.

We conclude that the prior art antibodies, in contrast to human/mouse equivalent anti-MET antibodies, elicit different biological activities in human and mouse systems.

### Example 15: Plasma Half-Life of Human/Mouse Equivalent Anti-MET Antibodies

Next, we moved the selected human/mouse equivalent anti-MET antibodies forward to in vivo studies. As a preliminary analysis, we determined their peak and trough levels in mice. To this end, we injected affinity purified 71G3, 71D6 and 71G2 (in their mouse IgG/λ format) into 7 week-old female BALB/c mice (Charles River) by i.p. injection. A single bolus of 1 mg/kg or 10 mg/kg was injected and blood samples were taken from the tail vein at 3, 6, 12 and 24 hours post-injection. Blood samples were processed and antibody concentration in plasma was determined by ELISA. Standard 96-well plates were coated with human MET ECD (100 ng/well) as described in Example 1 and then exposed to increasing dilutions of mouse plasma to capture anti-MET antibodies. After repeated washing with PBS, the presence of anti-MET antibodies was revealed using a HRP-conjugated donkey anti-mouse antibody (Jackson Laboratories). To quantify bound antibody, we set up a standard curve of purified 71G3, 71D6 and 71G2 in the same conditions.

The antibody concentrations in plasma were similar for all the antibodies tested and directly proportional to the amount of protein injected. After 24 hours, antibody concentration in plasma was approximately 15 nM for the 1 mg/kg bolus and 250 nM for the 10 mg/kg bolus. Considering that the agonistic activity of these antibodies in the most demanding assay (the branching morphogenesis assay) reaches saturation at a concentration of 5 nM or lower, we can safely conclude that the plasma levels of antibodies obtained by i.p. injection are relevant from a biologic viewpoint with boluses as low as 1 mg/kg.

Furthermore, we also calculated the plasma half-life of injected antibodies. This was achieved by transforming the antibody concentration to natural logarithm (Ln), fitting the data into a line and then calculating the slope of the line. This analysis led to estimate that the half-lives of 71G3, 71D6 and 71G2 are very similar and correspond approximately to 3 days for the 1 mg/kg bolus and 9 days for the 10 mg/kg bolus. This is a significantly higher stability compared to that of recombinant HGF which has been reported to have a half-life of 2.4 minutes in rodents (Ido et al., Hepatol Res. 30, 175-181, 2004). The whole panel of plasma stability data is summarized in Table 23.

These data suggest that human/mouse equivalent anti-MET antibodies could advantageously substitute recombinant HGF in all clinical applications that require systemic administration of HGF.

TABLE 23
Plasma stability of human/mouse equivalent antibodies.

| | 1 mg/kg bolus | | 10 mg/kg bolus | |
|---|---|---|---|---|
| | Conc. after | Plasma | Conc. after | Plasma |
| mAb | 24 h (nM) | half-life (days) | 24 h (nM) | half-life (days) |
| 71G3 | 16.6 ± 1.6 | 2.917 | 251.7 ± 24.0 | 9.025 |
| 71D6 | 15.6 ± 1.6 | 3.040 | 246.9 ± 44.3 | 10.697 |
| 71G2 | 18.1 ± 0.6 | 3.282 | 262.2 ± 17.6 | 9.025 |

A single bolus (1 mg/kg or 10 mg/kg) of affinity purified 71G3, 71D6 and 71G2 was administered to 7 week-old female BALB/c mice by i.p. injection. Blood samples were taken from the tail vein at 3, 6, 12 and 24 hours post-injection, and antibody concentration in plasma was determined by ELISA. Plasma half-life was calculated by linear fitting of the natural logarithm transforms of antibody concentrations.

### Example 16: Fine Epitome Mapping

In order to finely map the epitopes of MET recognized by human/mouse equivalent anti-MET antibodies we pursued the following strategy. We reasoned that, if an antibody generated in llamas and directed against human MET cross-reacts with mouse MET, then this antibody probably recognizes a residue (or several residues) that is (or are) conserved between H. sapiens and M. musculus but not among H. sapiens, M. musculus and L. glama. The same reasoning can be extended to R. norvegicus and M. fascicularis.

To investigate along this line, we aligned and compared the amino acid sequences of human (UniProtKB #P08581; aa 1-932), mouse (UniProtKB #P16056.1; aa 1-931), rat (NCBI #NP_113705.1; aa 1-931), cynomolgus monkey (NCBI #XP_005550635.2; aa 1-948) and llama MET (GenBank #KF042853.1; aa 1-931) among each other. With reference to Table 12, we concentrated our attention within the regions of MET responsible for binding to the 71D6, 71C3, 71D4, 71A3 and 71G2 antibodies (aa 314-372 of human MET) and to the 76H10 and 71G3 antibodies (aa 546-562 of human MET). Within the former region of human MET (aa 314-372) there are five residues that are conserved in human and mouse MET but not in llama MET (Ala 327, Ser 336, Phe 343, Ile 367, Asp 372). Of these, four residues are also conserved in rat and cynomolgus monkey MET (Ala 327, Ser 336, Ile 367, Asp 372). Within the latter region of human MET (aa 546-562) there are three residues that are conserved in human and mouse MET but not in llama MET (Arg 547, Ser 553, Thr 555). Of these, two residues are also conserved in rat and cynomogus monkey MET (Ser 553 and Thr 555).

Using human MET as a template, we mutagenized each of these residues in different permutations, generating a series of MET mutants that are fully human except for specific residues, which are llama. Next, we tested the affinity of selected SEMA-binding mAbs (71D6, 71C3, 71D4, 71A3, 71G2) and PSI-binding mAbs (76H10 and 71G3) for these MET mutants by ELISA. To this end, the various MET proteins were immobilized in solid phase (100 ng/well in a 96-well plate) and then exposed to increasing concentrations of antibodies (0-50 nM) solution. As the antibodies used were in their human constant region format, binding was revealed using HRP-conjugated anti-human Fc secondary antibody (Jackson Immuno Research Laboratories). Wild-type human MET was used as positive control. The results of this analysis are presented in Table 24.

TABLE 24
The epitopes of MET responsible for agonistic antibody binding
represent residues conserved among H. sapiens, M. musculus,
R. norvegicus, M. fascicularis but not among the same species
and L. glama. The relevance of residues conserved among
human, mouse, rat, cynomolgus monkey but not llama MET for
binding to agonistic mAbs was tested by ELISA. Wild-type
(WT) or mutant (MT) human MET ECD was immobilized in solid
phase and exposed to increasing concentrations of mAbs in
solution. Binding was revealed using anti-human Fc secondary
antibodies. All binding values were normalized to the WT protein
and are expressed as % binding (E_{MAX}) compared to WT MET.

| | | mAb binding (% WT MET ECD) | | | | | | |
|---|---|---|---|---|---|---|---|---|
| | MUTA- | SEMA BINDERS | | | | | PSI BINDERS | |
| MT | TIONS | 71D6 | 71C3 | 71D4 | 71A3 | 71G2 | 76H10 | 71G3 |
| WT | — | 100.0 | 100.0 | 100.0 | 100.0 | 100.0 | — | — |
| A | 1, 2, 3 | 103.3 | 99.8 | 114.5 | 116.8 | 92.1 | — | — |
| B | 4, 5 | 0.0 | 0.0 | 0.0 | 0.0 | 0.0 | — | — |
| C | 1, 2, 3, 4, 5 | 0.0 | 0.0 | 0.0 | 0.0 | 0.0 | — | — |
| D | 1, 2 | 128.0 | 101.8 | 119.6 | 127.9 | 113.5 | — | — |
| E | 2, 3, 4 | 43.6 | 59.6 | 57.2 | 65.4 | 41.4 | — | — |
| F | 2, 4, 5 | 0.0 | 0.0 | 0.0 | 0.0 | 0.0 | — | — |
| G | 3, 4, 5 | 0.0 | 0.0 | 0.0 | 0.0 | 0.0 | — | — |
| H | 2, 4 | 38.6 | 61.6 | 58.7 | 76.7 | 40.2 | — | — |
| I | 6, 7, 8 | — | — | — | — | — | 100.0 | 100.0 |
| J | 6, 7 | — | — | — | — | — | 89.0 | 91.2 |
| K | 6, 8 | — | — | — | — | — | 0.0 | 0.0 |
| L | 7, 8 | — | — | — | — | — | 0.0 | 0.0 |

The results presented above provide a definite and clear picture of the residues relevant for binding to our agonistic antibodies.

All the SEMA binders tested (71 D6, 71C3, 71 D4, 71A3, 71 G2) appear to bind to an epitope that contains 2 key amino acids conserved in human, mouse, cynomolgus and rat MET but not in llama MET lying within blade 5 of the SEMA β-propeller: Ile 367 and Asp 372. In fact, mutation of Ala 327, Ser 336 or Phe 343 did not affect binding at all; mutation of Ile 367 partially impaired binding; mutation of lie 367 and Asp 372 completely abrogated binding. We conclude that both Ile 367 and Asp 372 of human MET are important for binding to the SEMA-directed antibodies tested.

Also the PSI binders tested (76H10, 71G3) appear to bind to a similar or the same epitope. In contrast to the SEMA epitope, however, the PSI epitope contains only one key amino acid also conserved in human, mouse, cynomolgus and rat MET but not in llama MET: Thr 555. In fact, mutation of Arg 547 or Ser 553 did not affect binding at all, while mutation of Thr 555 completely abrogated it. We conclude that Thr 555 represents the crucial determinant for binding to the PSI-directed antibodies tested.

### Example 17: MET Agonist Antibodies Promote Langerhans Islet Growth and Pancreatic Beta Cell Regeneration in Healthy Mice

In order to assess the biological effect of a MET agonistic antibody on pancreatic beta cells in vivo, we subjected both male and female adult BALB/c mice (Charles River) to systemic treatment with 0, 3, 10 or 30 mg/kg purified 71D6 antibody for a period of three months (6 mice per gender per group for a total of 48 animals). Antibody was administered 2 times a week by i.p. injection. Body weight and fasting blood glucose concentration was measured every month throughout the experiment. At the end of the 3 month period, mice were sacrificed; pancreas were collected, embedded in paraffin and processed for histological analysis. Sections were stained with hematoxylin and eosin, examined by microscopy and photographed. Images were analyzed using ImageJ software (National Institutes of Health) to determine Langerhans islet number and size.

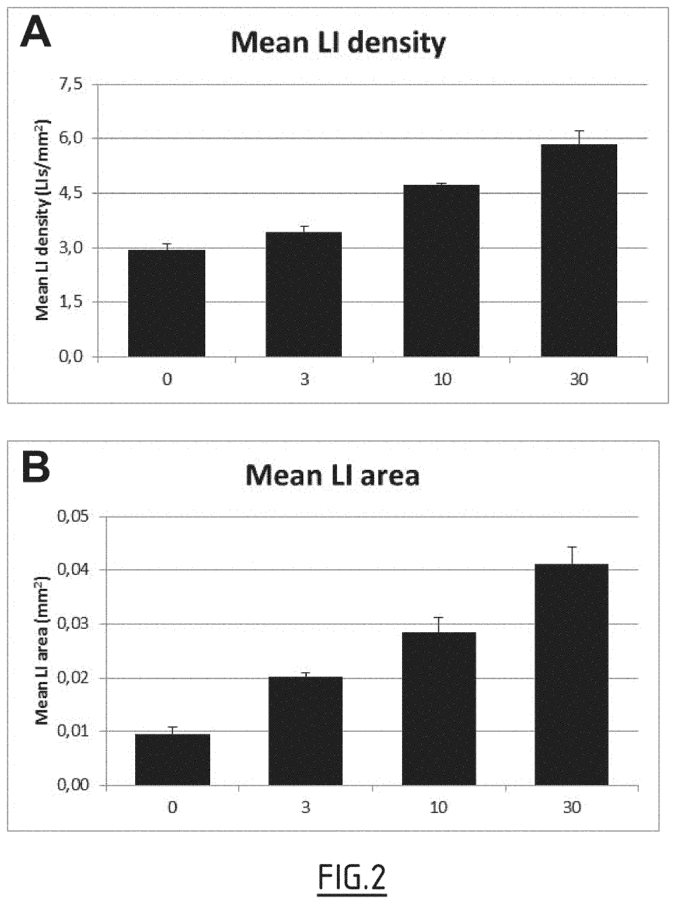

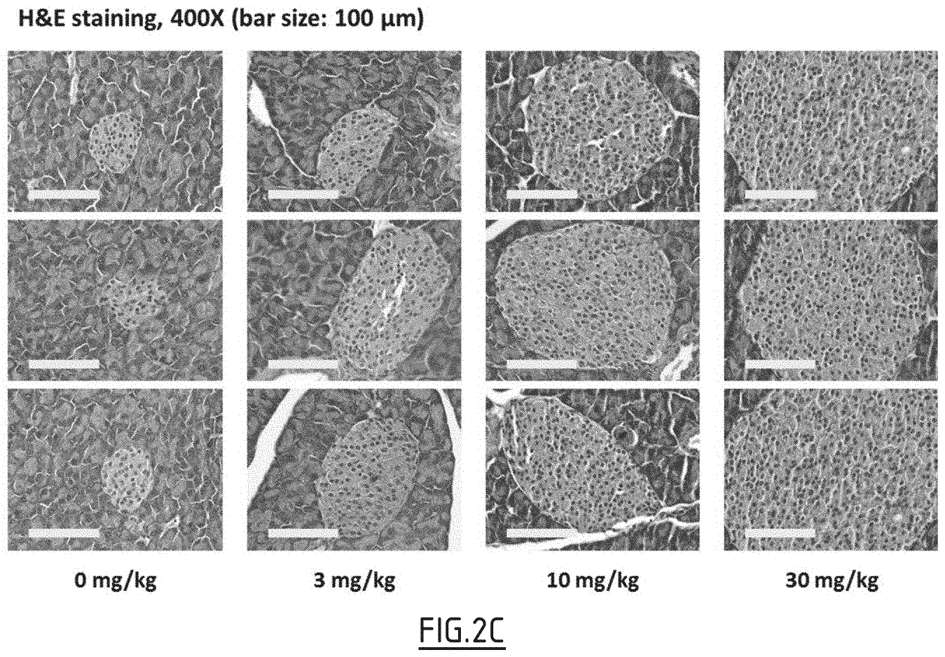

Chronic treatment with 71D6 did not affect total body weight in either male or female animals (FIG. 1A). Likewise, basal glycemia measured in fasting animals did not change at any antibody dose (FIG. 1B). On the other hand, histological analysis of pancreatic sections revealed that treatment with 71D6 agonistic antibody significantly increased the number of Langerhans islets in a dose-dependent fashion (FIG. 2A). In untreated, control animals (0 mg/kg), the number of islets per unit of pancreas section (mm²) was approximately 3. At the maximal dose tested (30 mg/kg), islet number per mm² reached a value of 6; at 3 and 10 mg/kg islet density displayed intermediate values. Treatment with 71D6 also significantly increased Langerhans islet size (FIG. 2B). In control animals, the mean islet size was approximately 0.01 mm² (expressed as the area of the islet section, as measured by microscopic imaging on tissue section stained with hematoxylin and eosin). At a dose of 3 mg/kg, mean islet area increased 2 times compared to 0 mg/kg; at a dose of 10 mg/kg, it increased 3 times compared to control; at 30 mg/kg, islets were 4 times bigger compared to untreated animals. Representative images of pancreas sections stained with hematoxylin and eosin are shown in FIG. 2C.

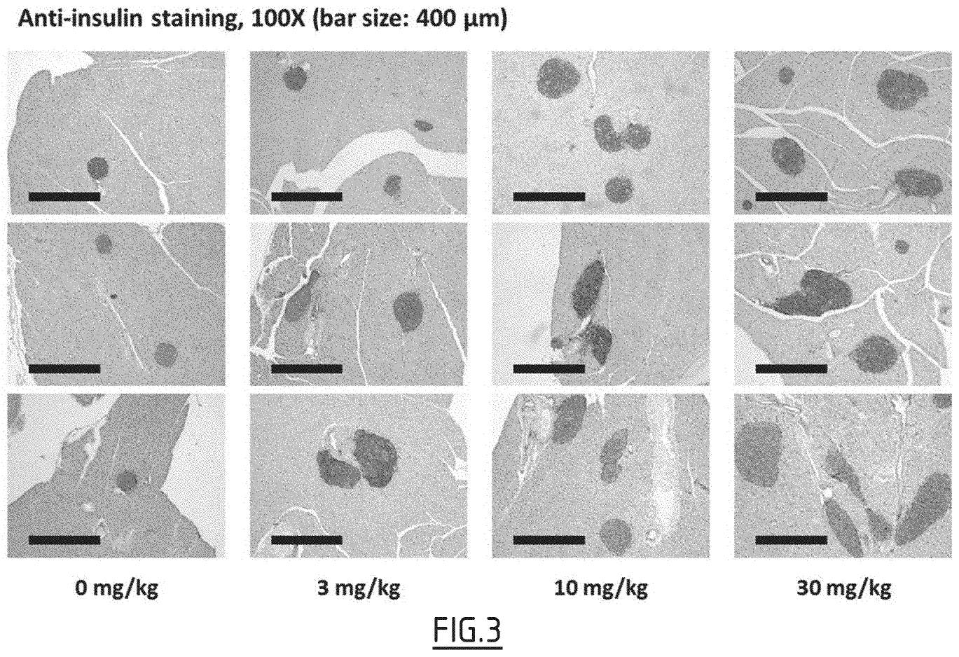

Interestingly, immunohistochemical analysis with anti-insulin antibodies revealed that treatment with 71D6 results in expansion of the pancreatic beta cell population and in potentiation of insulin expression (FIG. 3). This finding suggests that 71D6-induced size increase of Langerhans islets is due to hyperproliferation of pancreatic beta cells. Furthermore, potentiated insulin expression demonstrates that these beta cells are healthy and functional. Altogether, these results indicate that 71D6 acts as a mitogenic and pro-regenerative factor for pancreatic beta cells in vivo.

### Example 18: MET Agonist Antibodies Promote Langerhans Islet Growth and Pancreatic Beta Cell Regeneration in a Mouse Model of Type 1 Diabetes Mellitus

Prompted by the observation that agonistic anti-MET antibodies act as mitogenic factors for beta cells, we tested their therapeutic potential in a mouse model of type 1 diabetes. Ablation of pancreatic beta cells was achieved in mice by administration of multiple, low doses of streptozotocin (STZ; a chemical agent that selectively kills beta cells and a standard compound used to induce type 1 diabetes mellitus in laboratory animals).

STZ was injected i.p. into female BALB-c mice (Charles River) at a dose of 40 mg/kg every 24 hours for 5 consecutive days. One week after the last injection, STZ-treated mice displayed a mean basal glycemia two times higher compared to untreated mice (240 mg/dL vs. 120 mg/dL), suggesting that the chemical compound had efficiently killed beta cells. At this point, mice were randomized into 4 arms of 7 mice each based on basal glycemia, which received treatment with (i) vehicle only (PBS), (ii) purified 71D6 antibody, (iii) purified 71G2 antibody, (iv) purified 71G3 antibody. Antibodies were administered at a dose of 1 mg/kg two times a week by i.p. injection. An additional, fifth arm contained 7 mice that received no STZ or antibody and served as a healthy control. The experiment continued for 8 weeks; basal glycemia was monitored throughout the experiment. At the end of the 8 week period, mice were sacrificed and subjected to autopsy. Blood was collected for analysis; pancreases were extracted, processed for histology and embedded in paraffin.

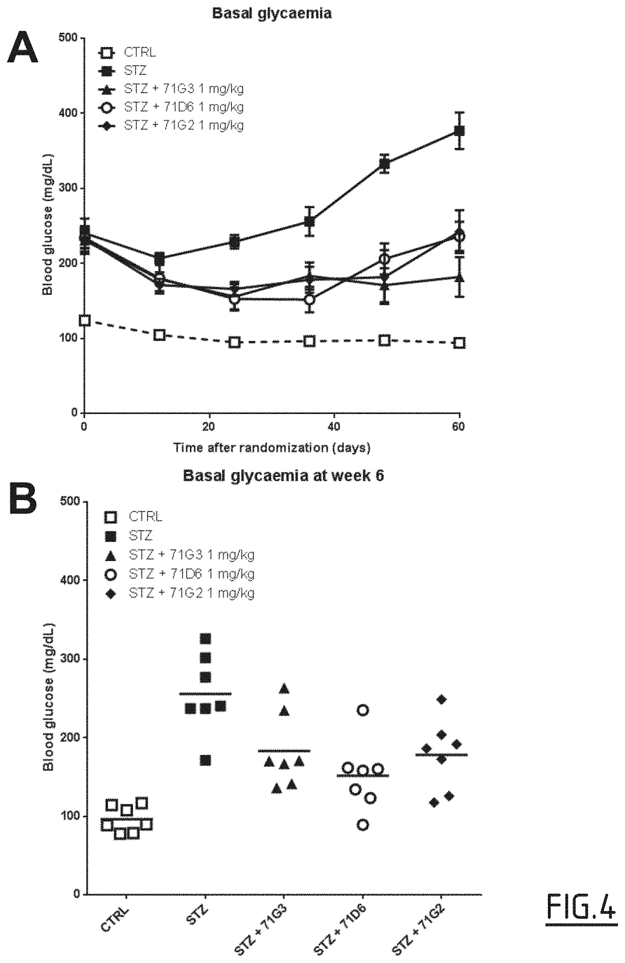

As shown in FIG. 4A, basal blood glucose levels in STZ-treated mice continued to increase over time. This is consistent with the notion that STZ-induced beta cell damage causes chronic pancreas inflammation, leading to progressive aggravation of organ injury. Interestingly, antibody administration did not completely normalize glycemia, but significantly lowered it towards more normal levels. Six weeks after treatment start (i.e. 7 weeks after last STZ injection), mice treated with STZ only displayed a mean basal glycemia of approximately 250 mg/dL; mice treated with STZ and 71D6 had a mean basal glycemia of approximately 150 mg/dL; mice treated with STZ and 71G2 or 71G3 displayed a slightly higher glycemia, but still significantly lower than the STZ alone arm; control untreated mice showed a mean basal glycemia of 96 mg/dL (FIG. 4B).

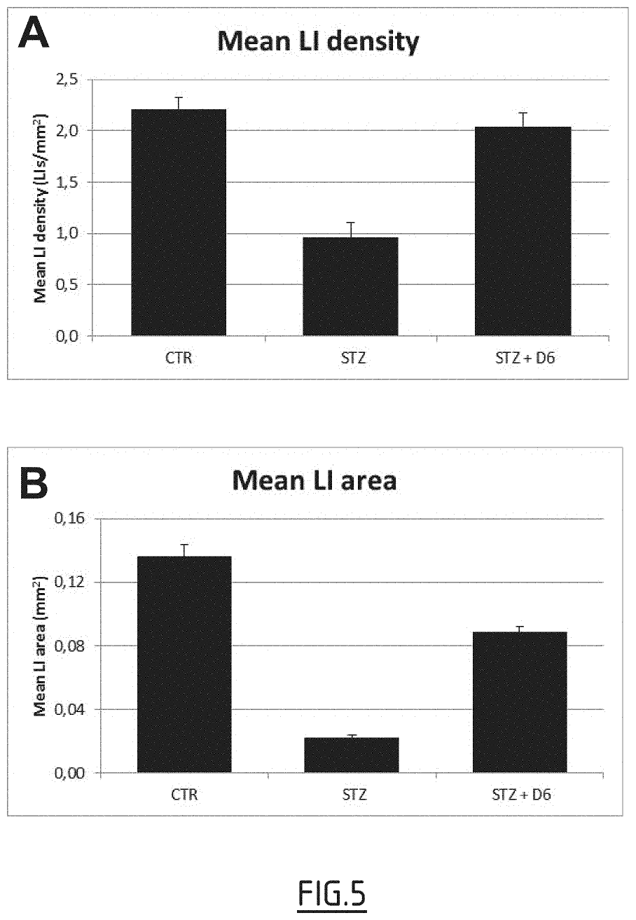

In order to determine the effect of MET agonist antibodies on Langerhans islets, pancreas sections were stained with hematoxylin and eosin and analysed by microscopy. Digital images of Langerhans islets were analysed using ImageJ software (National Institutes of Health). The number, density and size of Langerhans islets were determined by digital data analysis. As shown in FIG. 5A, STZ administration dramatically reduced the number of Langerhans islets in the pancreas of mice treated with this compound only. In contrast, animals treated with STZ and 71D6 displayed a more normal Langerhans islet density, very similar to that observed in untreated, control mice. STZ treatment also heavily affected Langerhans islet size, reducing it by more than 6 times (FIG. 5B). Remarkably, 71D6 antagonized this reduction, limiting it to 1.5 times. Similar results were obtained with 71G2 and 71G3, although with similar but slightly reduced potency (71D6>71G2>71G3). Representative images of pancreas sections stained with hematoxylin and eosin are shown in FIG. 5C.

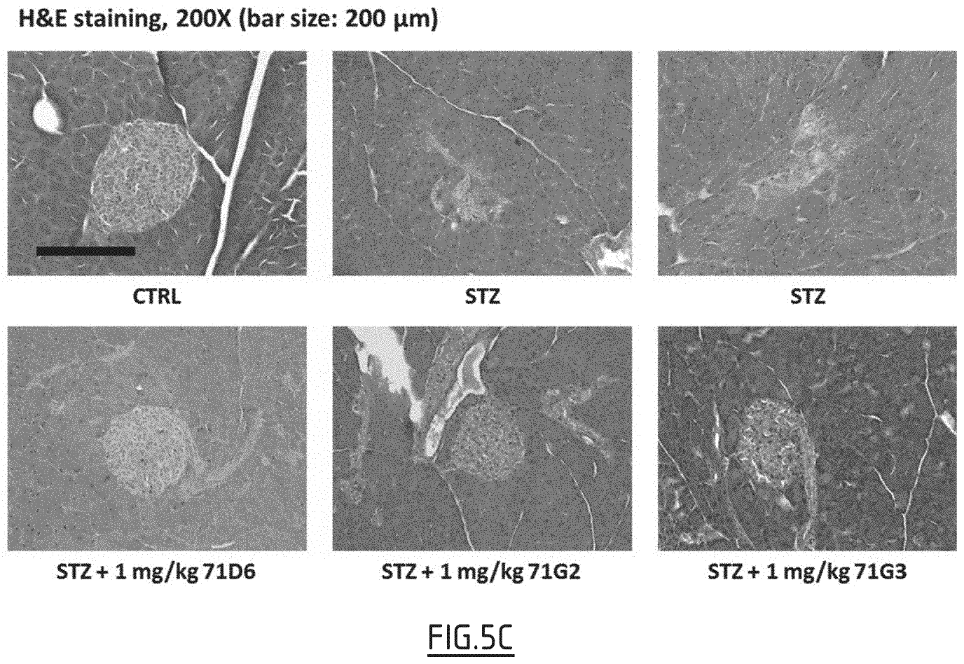

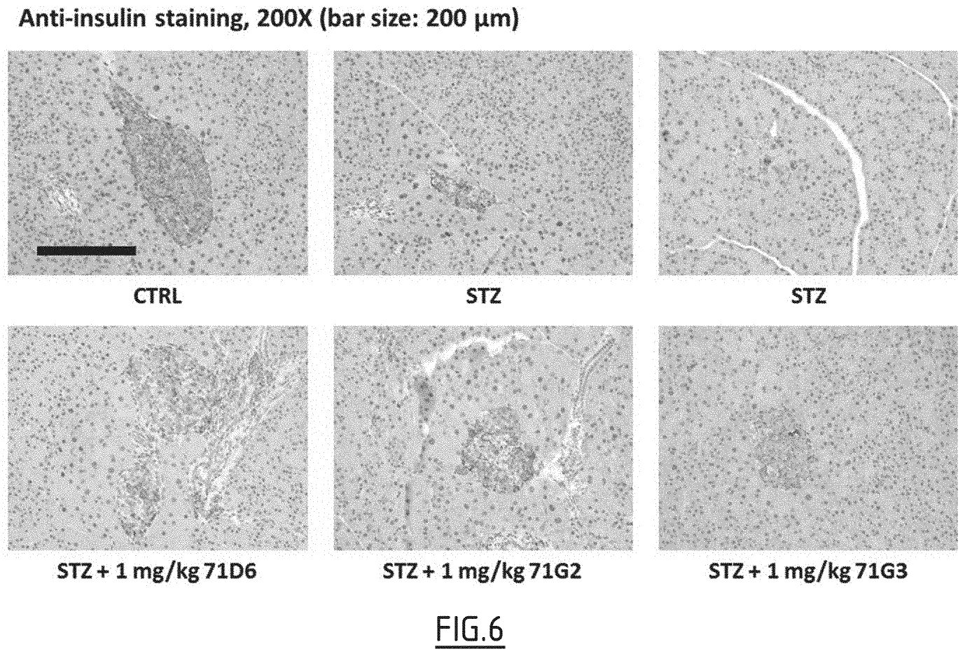

Pancreas sections were further analysed by immunohistochemistry using anti-insulin antibodies. This analysis revealed that STZ not only reduced Langerhans islet number and size, but also dramatically curtailed beta cells and, as a consequence, insulin production. Again notably, MET agonist antibody treatment rescued beta cells from STZ-induced destruction and maintained insulin production elevated. This may explain the lower levels of blood glucose observed in animals treated with both STZ and MET agonist antibodies compared to mice receiving STZ only. Representative images of pancreas sections stained with anti-insulin antibodies are shown in FIG. 6.

### Example 19: MET Agonist Antibodies Promote Langerhans Islet Growth and Pancreatic Beta Cell Regeneration in a Mouse Model of Type 2 Diabetes Mellitus

Prompted by the observation that anti-MET agonistic antibodies induce pancreatic beta cells regeneration in healthy mice and in a type 1 diabetes mellitus model, we set to test their therapeutic potential further in other related indications. Although characterized by different etiological mechanisms, type 2 diabetes also leads to Langerhans islet degeneration. In fact, type 2 diabetes is characterized by hyperinsulinemia in the presence of insulin resistance, leading to high blood glucose levels and inability of beta cells to compensate for the increased demand of insulin (Christoffersen et al., Am J Physiol Regul Integr Comp Physiol 297:1195-201, 2009). Therefore, regeneration of beta cells is also an unmet medical need for type 2 diabetes mellitus patients.

In order to explore the therapeutic potential of agonistic MET antibodies in type 2 diabetes, we selected the db/db obese mouse model. Due to a mutation in the leptin gene, these animals are hyperphagic, obese, hyperinsulinemic and hyperglycemic. Obesity is evident from 3-4 weeks of age, with hyperinsulinemia becoming apparent at around week 2 and hyperglycaemia developing between weeks 4 and 8. Female db/db mice were obtained from Charles River at the age of 7 weeks. One week later, animals were randomized into 4 arms of five mice each, which received treatment with (i) vehicle only (PBS), (ii) purified 71D6 antibody, (iii) purified 71G2 antibody, (iv) purified 71G3 antibody. Antibodies were administered at a dose of 1 mg/kg two times a week by i.p. injection. Considering that the background strain of db/db mice is C57BL6/J, we used these mice as healthy control animals. Basal glycemia was monitored throughout the experiment. After 8 weeks of treatment (16 weeks of age), mice were sacrificed and subjected to autopsy. Pancreases were collected, processed for histology and embedded in paraffin. Tissue sections were stained with hematoxylin and eosin in order to visualize Langerhans islets. Beta cells and insulin production were highlighted by immunohistochemical analysis using anti-insulin antibodies.

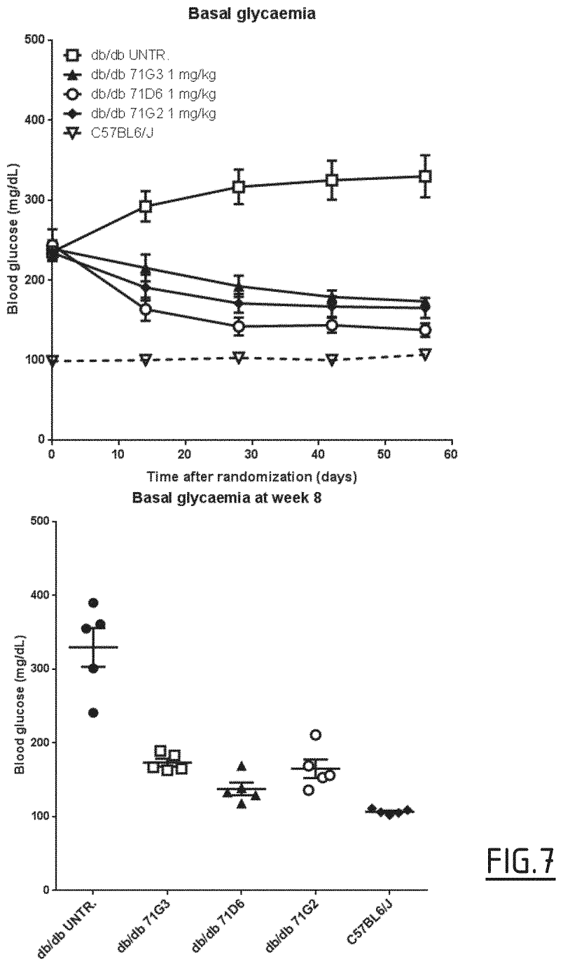

As shown in FIG. 7A, untreated db/db mice already displayed a fairly advanced hyperglycemia at week 7 of age (approximately 240 mg/dL). Thereafter, blood glucose levels steadily increased until they reached a plateau exceeding 300 mg/dL. Interestingly, animals treated with 71D6, 71G2 and 71G3 displayed a significantly lower glycemia throughout the experiment, although not matching that of control C57BL6/J control mice. At the end of the experiment, db/db untreated mice had a basal glycemia of approximately 330 mg/dL; 71D6-treated db/db animals showed instead a mean basal glycemia of approximately 140 mg/dL; 71G2- and 71G3-treated animals displayed a basal glycemia of approximately 180 mg/dL (FIG. 7B).

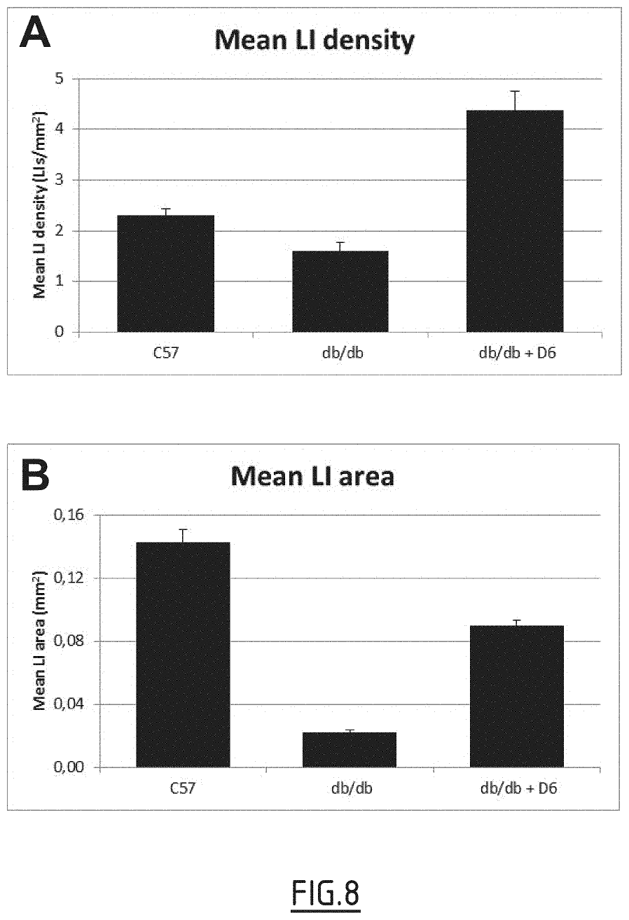

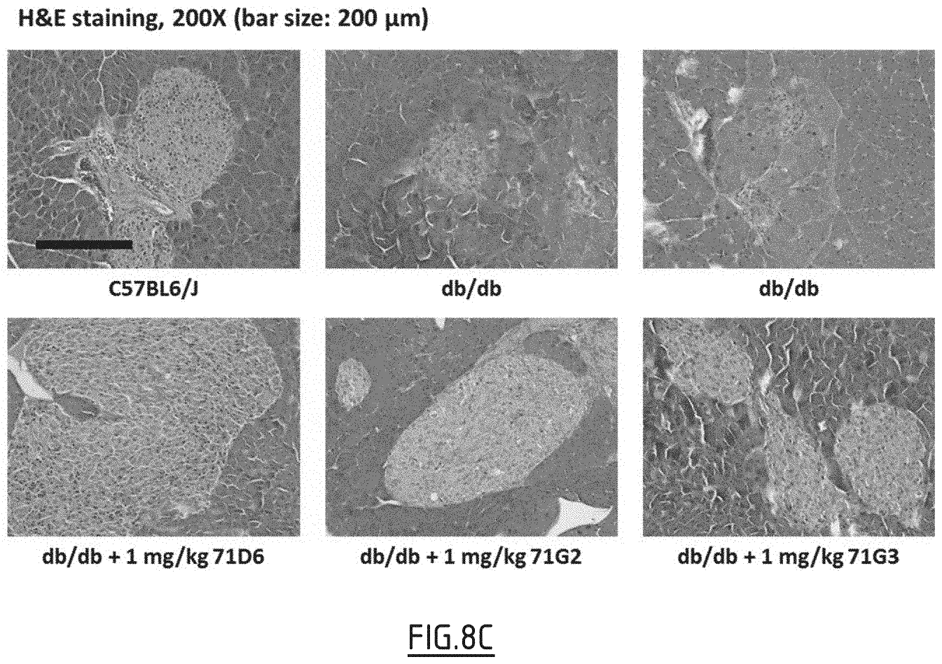

Pancreas sections stained with hematoxylin and eosin were analysed by microscopy and photographed. Langerhans islets were analysed using ImageJ software to estimate islet number, density and size. This analysis revealed that Langerhans islets are extremely degenerated in db/db mice at 16 weeks of age compared to age-matching C57BL6/J controls, both in terms of number and size. In fact, C57BL6/J mice displayed a mean pancreatic islet density of 2.3 islets/mm², while untreated db/db mice showed a density of 1.6 islets/mm² (FIG. 8A). Strikingly, islet density dramatically increased in db/db mice treated with 71D6, reaching values significantly higher than those observed in healthy controls (4.4 islets/mm²). Islet size was also heavily impaired in db/db mice (FIG. 8B) compared to C57BL6/J controls. In the latter strain, Langerhans islets had a mean area of 0.3 mm², which was reduced by approximately 10 times in untreated db/db mice. Strikingly, 71D6 treatment completely rescued islet size decrease, bringing it back to values similar or even greater than those characteristic of C57BL6/J healthy animals. Similar results with respect to both islet number and size were obtained with 71G2 and 71G3, although with slightly reduced potency (71D6>71G2>71G3). Representative images of pancreas sections stained with hematoxylin and eosin are shown in FIG. 8C.

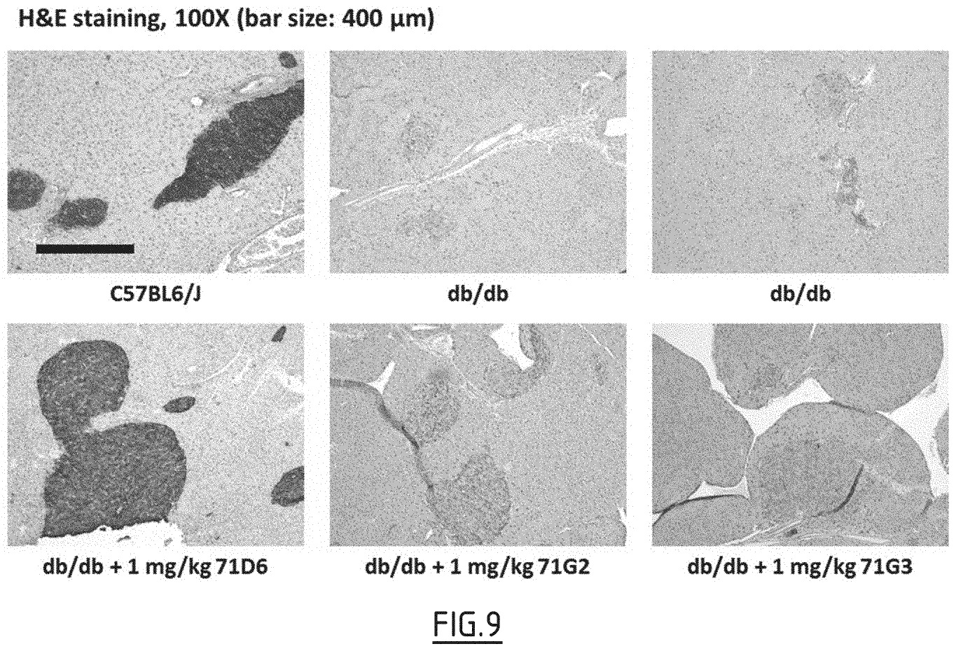

We further characterized the biological effects of 71D6 by assessing its ability to specifically affect the beta cell population. To this end, pancreas sections were analysed by immunohistochemistry using anti-insulin antibodies. This analysis revealed that the few surviving islets in db/db mice contained very few insulin-expressing beta cells compared to healthy controls (FIG. 9). In contrast, db/db mice treated with 71D6, 71G2 or 71G3 contained significantly more functional beta cells, and these cells expressed much higher levels of insulin. This was particularly evident in the 71D6 arm, confirming that this antibody is more potent than 71G2 and 71G3.

These results as well as those presented in the previous Examples demonstrate that the 71D6, 71G2 and 71G3 MET agonistic antibodies promote beta cell survival and regeneration, contributing to maintaining normal levels of insulin. Considering that restoring functional beta cells significantly improves the symptoms of diabetes and the quality of life of diabetes patients, we suggest that agonistic anti-MET antibodies could represent an innovative tool for diabetes treatment in the clinic.

Importantly, a key requisite for moving MET agonistic antibodies forward to the clinic is their complete cross-reactivity with pre-clinical species, including rodents and non-human primates. In fact, we were able to demonstrate therapeutic activity of 71D6, 71G2 and 71G3 in mice because they maintain full cross-reactivity between human and mouse MET. Furthermore, 71D6 elicits exactly the same biological activity and potency in tissues of human, mouse, rat and monkey origin. Without this species equivalency it would be impossible to move the described MET agonist antibodies on towards first-in-human experimentation. Mainly due to this reason (i.e. absence of equivalency in preclinical species), the any agonistic MET antibodies known in the prior art could not be tested in preclinical models and lack therefore the necessary proof-of-efficacy.

Further along this avenue, another approach to treat both type 1 and type 2 diabetes mellitus is represented by pancreas transplantation, either as a whole organ or using isolated Langerhans islets or purified beta cells (Kieffer et al., J Diabetes Investig. 2017, epub ahead of print; doi: 10.1111/jdi.12758). This approach also has some limitations, particularly with respect to poor grafting and scarce survival of transplanted beta cells in the recipient. Given the potent ability of MET agonist antibodies described herein to promote beta cell regeneration and insulin secretion, they may also improve the efficacy of pancreatic tissue transplantation and amplify the beta cell population in graft-receiving patients.

### Example 20: MET Agonist Antibodies Preserve Pancreatic Beta Cell Function, Prevent Diabetes Onset and Cooperate with Immune-Suppressing Drugs in a Mouse Model of Autoimmune Type 1 Diabetes Mellitus

Type 1 diabetes mellitus is characterized by autoimmune-mediated destruction of pancreatic beta cells, leading to insufficient insulin secretion and inability of tissues to uptake blood glucose. Auto-antibody-mediated beta cell destruction begins earlier than the hyperglycemic phenotype manifests. At the time insulin-dependent diabetes is diagnosed, typically during adolescence, beta cell destruction may be already advanced, with only a minor fraction of the original beta cells surviving. Furthermore, beta cell destruction proceeds very rapidly, thus leaving a narrow window for therapeutic intervention after diagnosis.

Immuno-suppressive drugs are being investigated as therapy for newly-diagnosed type 1 diabetes patients, in an effort to reduce autoimmune-mediated islet cell destruction. However, immunosuppressants require several months before showing the first clinical benefits. When this occurs, approximately half year after treatment start, the beta cells of the pancreas continue to be destroyed, often completely. As a result, the efficacy of immunosuppressants is severely blunted if not nullified. Maintaining islet beta cells alive—or even better regenerating them—during this crucial window is a highly unmet medical need for diabetes patients.

In order to test whether MET-agonistic antibodies could antagonize immune-mediated beta cell destruction and cooperate with immune-targeting drugs in the context of type 1 diabetes, we selected an appropriate mouse model. NOD/ShiLtJ strain (commonly called NOD) is a polygenic model for autoimmune type 1 diabetes. Diabetes in NOD mice is characterized by hyperglycemia and leukocytic infiltration of the pancreatic islets. Marked decreases in pancreatic insulin content occur in females at about 12 weeks of age and several weeks later in males. NOD mice are considered the type 1 diabetes animal model that best reproduces the pathology observed in humans. In this strain, several studies have been conducted with immunosuppressants for studying their potential in ameliorating hyperglycemia and/or delaying diabetes onset. In particular, antibodies directed against the lymphocyte-specific surface marker CD3 have been shown to be particularly effective in several studies (Chatenoud et al. Proc Natl Acad Sci USA 91:123-127, 1994; Chatenoud et al. J Immunol 158:2947-2954, 1997; Gill et al. Diabetes 65:1310-1316, 2016; Kuhn et al. Immunotherapy, 8:889-906, 2016; Kuhn et al. J Autoimmun 76:115-122, 2017). Interestingly, these studies showed that oral delivery of these immune-targeted antibodies gives rise to less side effects compared to systemic delivery. The most effective protocol consisted in treating mice for 5 consecutive days and then stopping the therapy (Ochi et al. Nat Med. 12:627-635, 2006). Notably, the therapeutic efficacy dramatically dropped when the oral drug dose exceeded 5 μg per mouse (0.25 mg/kg).

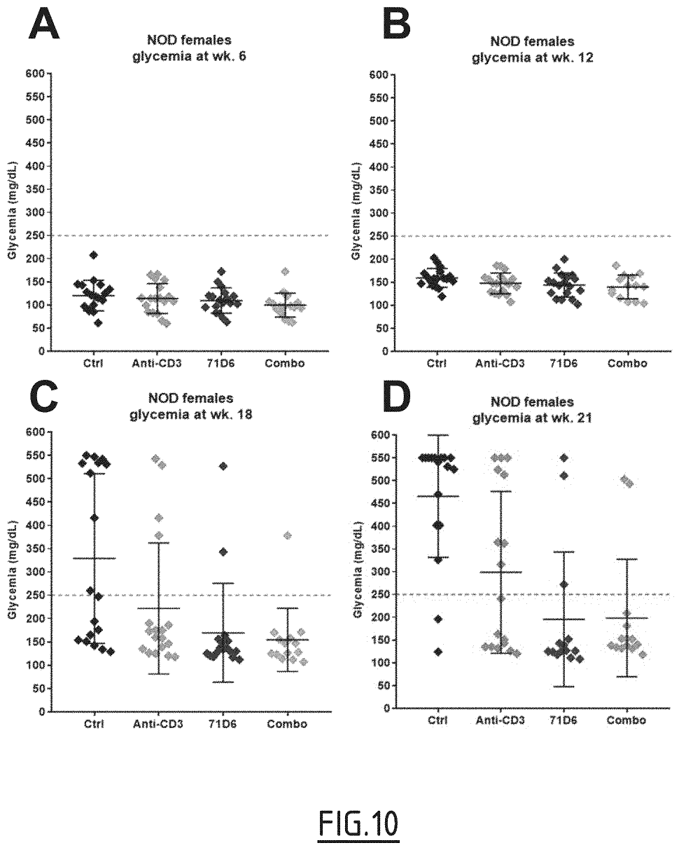

To test whether our agonistic anti-MET antibodies displayed a therapeutic effect and to investigate their potential cooperation with immune-targeting drugs, we obtained seventy-two 6-week-old female NOD mice from Charles River. Blood sugar was measured in random fed (i.e. not fasting) animals using test strips for human use (multiCare in; Biochemical Systems International). At this time, NOD mice displayed a pre-diabetic, average glycemia of approximately 110 mg/dL (FIG. 10A). Mice were randomized into four different arms of 18 animals each, making sure that all groups were as homogeneous as possible with respect to glycemia. Starting from week 7, the four arms were subjected to different treatments as follows: no drug (CONTROL); 0.15 mg/kg anti-CD3 antibody (CD3); 3 mg/kg purified 71D6 antibody (71D6); 0.15 mg/kg anti-CD3 antibody+3 mg/kg purified 71D6 antibody (COMBO). Anti-CD3 antibody was delivered orally by gavage in 100 μL of PBS one time per day for 5 consecutive days and then interrupted as per protocol. 71D6 was delivered i.p. in 200 μl of PBS two times per week for the whole duration of the experiment. Mice were fed ad libitum with a standard diet. Glycemia was measured on random fed animals one time per week using strips as above. An animal was considered diabetic if it showed a glycemic value greater than 250 mg/dL for 2 consecutive weeks.

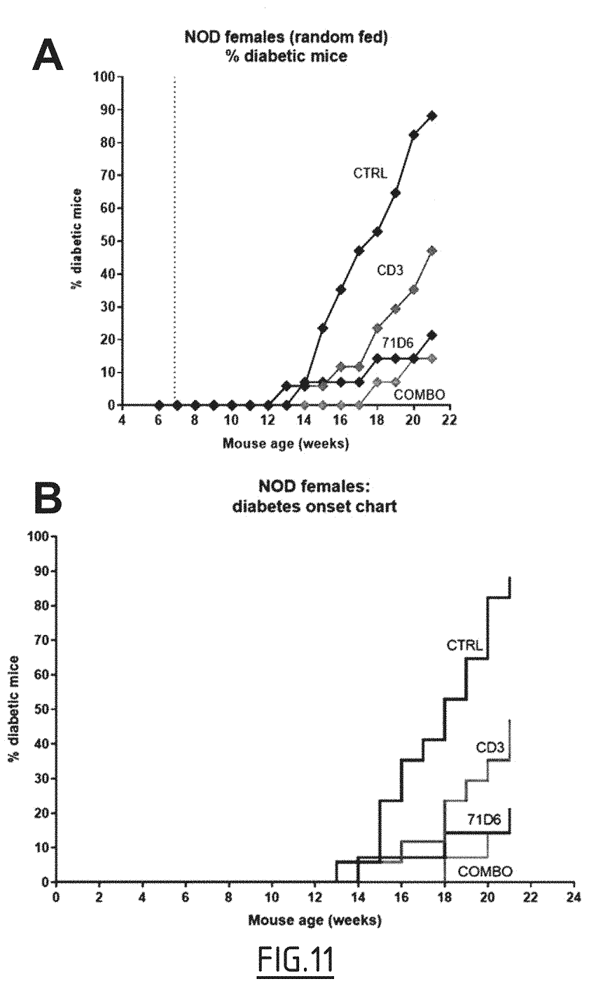

In line with the literature, no diabetic animal was recorded until week 12 (FIG. 10B). At week 13, diabetes started manifesting in the CONTROL and CD3 arms. At week 18, 50% of the CONTROL animals were diabetic (FIG. 10C), exactly as described by the original strain provider (The Jackson Lab—001976 mouse strain datasheet; https://www.jax.org/strain/001976). At week 21, when the experiment was interrupted, 88% of the CONTROL mice were diabetic, while the other arms displayed significant lower values: CD3, 47%; 71D6, 21%; COMBO, 14% (FIG. 10D). Analysis of diabetes onset overtime is shown in FIG. 11A. A Kaplan-Meier plot is shown in FIG. 11B. Statistical analysis was performed using Prism software (Graph Pad). A Mantel-Cox test, a Logrank test for trend and a Gehan-Breslow-Wilcoxon test all gave a p value of less than 0.001, indicating that the differences among curves are statistically significant.

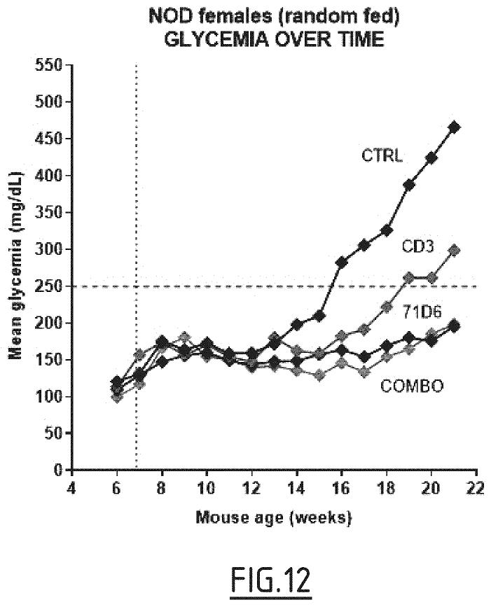

Average non-fasting glycemia increased constantly in all arms, but reached extremely high levels (>450 mg/dL) only in the CONTROL untreated arm (FIG. 12). Consistent with the diabetes onset data, blood sugar levels followed a precise order CONTROL>CD3>71D6>COMBO. During the course of the experiment (4 months), a few mice died for reasons independent of treatment, mainly in-cage fighting with fellow mice and bacterial infections (CONTROL, 1/18; CD3, 1/18; 71D6, 4/18; COMBO, 4/14). Since glycemia levels in single diabetic mice reached quickly extreme values (>550 mg/dL), animals were sacrificed three weeks after diabetes diagnosis. In these cases, a value of 550 mg/dL was used for computing the average glycemia of a group even after death. All mice, whether diabetic or not, where sacrificed at the end of week 21.

Before sacrifice, all mice were subjected to a glucose tolerance test (GTT). To this end, animals were food-starved overnight. The morning after, a blood sample was collected for glycemia and insulin measurement. A glucose solution (3 g/kg in 200 μL PBS) was injected i.p. and a second blood sample was collected 3 minutes later. Soon after, mice were sacrificed and the major organs were collected for analysis, including the liver and the pancreas. Blood glucose concentration was determined using strips as described above. Insulin concentration was measured with an Ultra-Sensitive Mouse Insulin ELISA Kit (Crystal Chem).

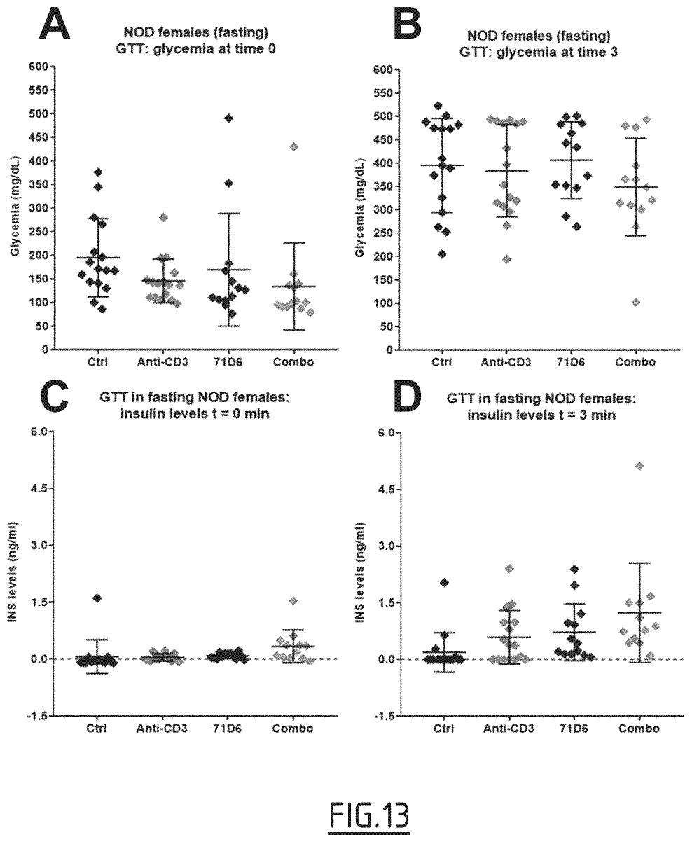

Blood sugar content analysis revealed the following scenario. At time zero, glycemia was lower in the treated arms compared to control (CONTROL>CD3>71D6>COMBO; FIG. 13A), but three minutes after glucose challenging it raised at similar levels in all groups (>350 mg/dL; FIG. 13B). In contrast, blood insulin concentration was very low at time zero, with the exception of the COMBO arm that showed slightly higher levels (FIG. 13C). Notably, following glucose injection, insulin levels appeared very different depending on the treatment arm, showing a reverse order (COMBO>71D6>CD3>CONTROL; FIG. 13D). Since NOD mice display a peculiar modulation of insulin in their pre-diabetic stage (Amrani et al. Endocrinology 139:1115-1124, 1998), it is difficult to directly compare these absolute values with other non-diabetic strains. In any case, we can certainly conclude that animals belonging to the treatment arms respond to glucose stimulation by secreting insulin, while control animals do not.

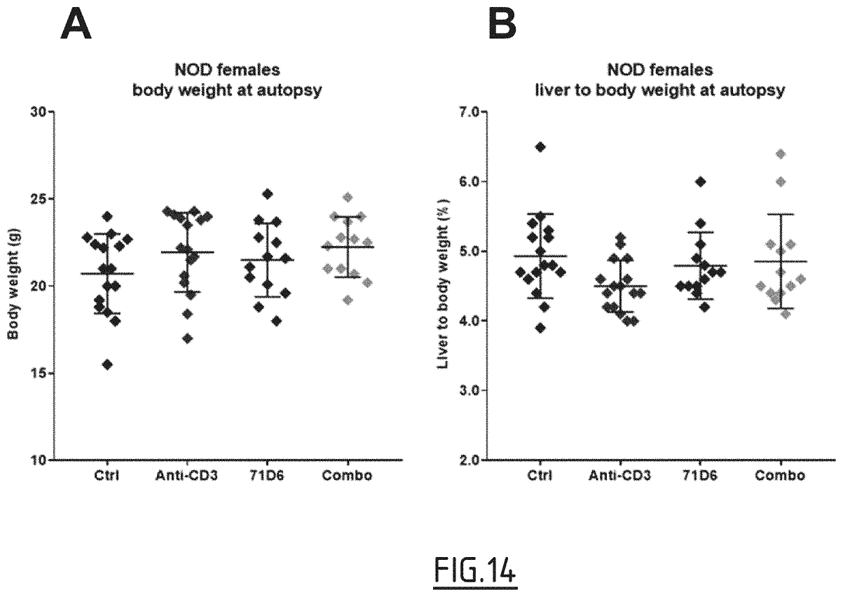

Consistent with an ameliorated diabetic phenotype, body weight was slightly (although not significantly) higher in the treatment arms compared to the control arm at the time of autopsy (FIG. 14A). There was no significant difference in liver to body weight in any of the group (FIG. 14B), suggesting that 71D6-mediated liver growth (observed in other mouse systems) is strain-specific. No other biological or pathological sign or tract was detected in 7106-treated animals while performing autopsy or when analyzing tissue histology.

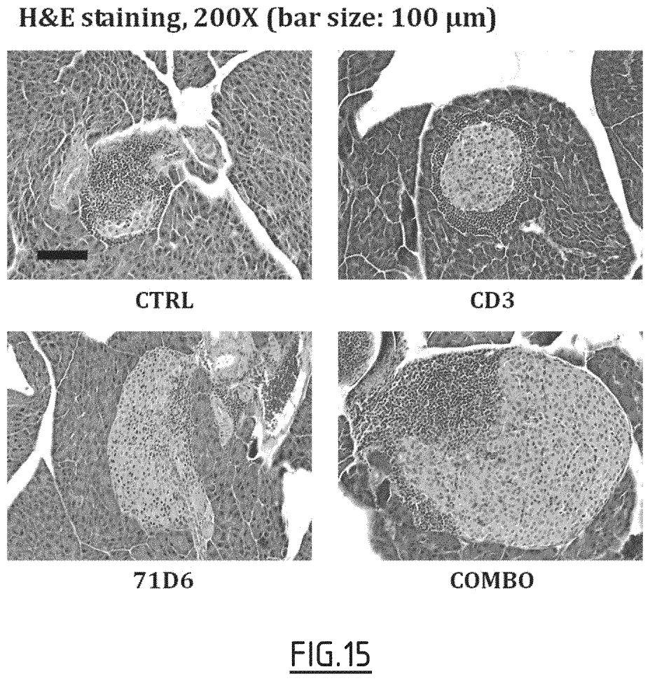

Pancreas samples were embedded in paraffin and processed for histological analysis. Tissue section were stained with hematoxylin and eosin and analyzed by microscopy. This analysis revealed that the pancreas of the majority of animals belonging to the CONTROL arm contained a very small number of Langerhans islets, and those islets that were visible were abnormally small and highly infiltrated with lymphocytes (FIG. 15). In contrast, Langerhans islets in the CD3 arm were more abundant and less degenerated, although still infiltrated with lymphocytic cells. Pancreas sections of the 71D6 arms contained more Langerhans islets compared to both CONTROL and CD3, and islet size was greater on average; however, lymphocyte infiltration was still evident. Finally, pancreatic islets of the COMBO arm were abundant and big, although infiltrated as well.

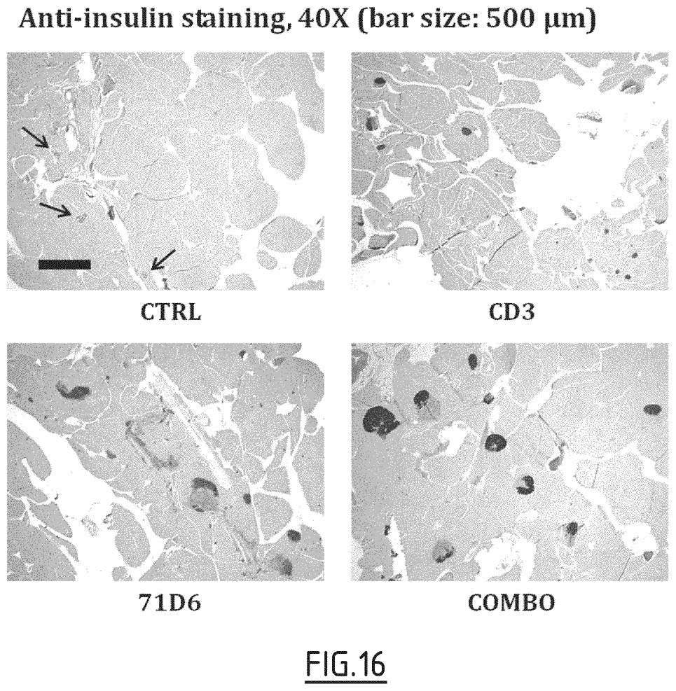

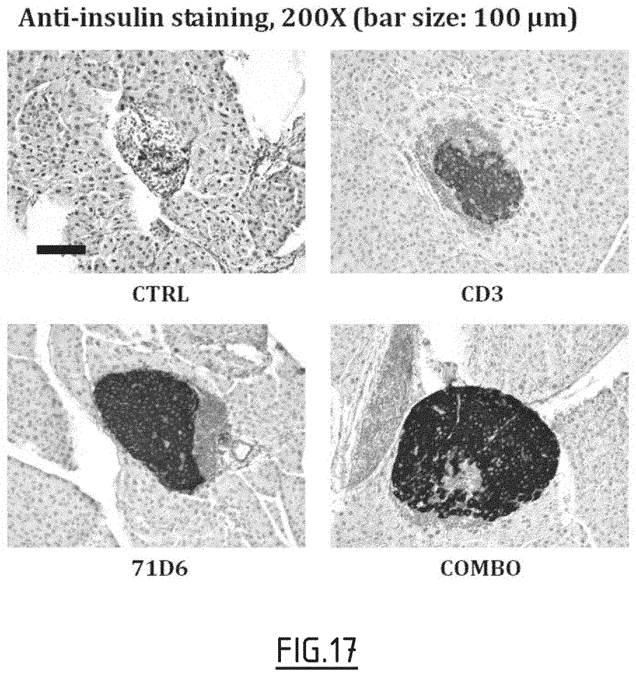

The major treatment-dependent differences were observed in pancreas sections stained with anti-insulin antibodies (FIG. 16). In the CONTROL arm, very little if any staining was observed in the few visible islets. In the CD3 arm, insulin signal was higher, although not as potent as observed in 716-treated animals. Islets found in the COMBO arm displayed the highest and most homogeneous insulin signal compared to all other arms. At higher magnification, these features could be appreciated in greater detail (FIG. 17). Islets in untreated animals contained very few if any insulin-producing cell. In contrast, the majority of islet cells in the CD3 arm were positive for insulin. In the 71D6 arm, islets were both large and intensively stained. Pancreas of the COMBO arm contained the largest and most insulin-producing islets among all groups.

As mentioned above, the number of insulin-producing beta cells within the Langerhans islets was clearly higher in the treated arms (COMBO>71D6>CD3>CONTROL). However, cell infiltration was very dishomogeneous, and no major differences with respect to the number of lymphocytic cells recruited around the islets could be observed among the various arms. This could be explained by two different mechanisms depending on the therapeutic agent. It is well established that oral delivery of anti-CD3 antibodies induces immunogenic tolerance rather than eliminating the immune response (Chatenoud et al. J Immunol 158:2947-2954, 1997). The tolerogenic process involves activation and proliferation of T-regulatory cells, which inhibit auto-antibody-mediated beta cell destruction (Chatenoud Novartis Found Symp 252:279-220, 2003). This explains why, in the CD3 arm, pancreatic beta cells are not destroyed in spite of immune cell infiltration. On the other hand, the data presented in the previous examples suggest that 71D6 promotes beta cell survival and regeneration. It can be therefore hypothesized that 71D6 both antagonizes immune-mediated beta cell death and promote beta cell growth, thus preserving beta cell mass despite heavy immune cell infiltration.

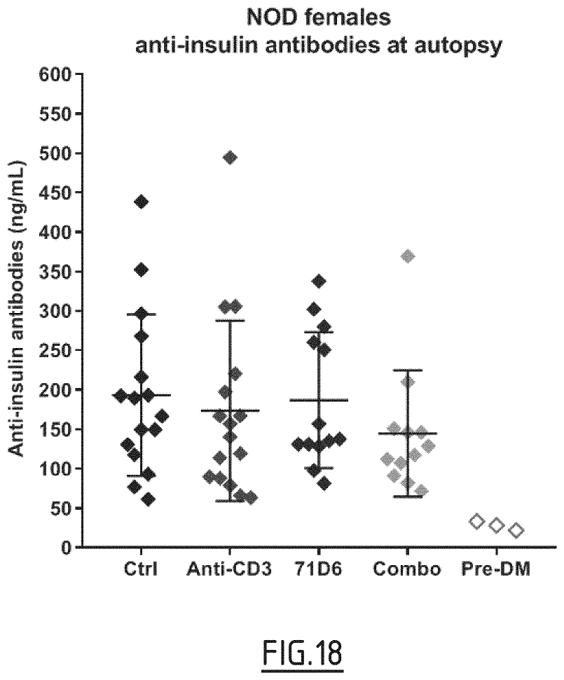

To further investigate the role of the immune system in the response to anti-CD3 and anti-MET antibodies, we measured anti-insulin antibodies in mouse plasma. To this end, plasma samples collected at autopsy from all mice as well as from young, pre-diabetic female NOD mice (week 7 of life) were analyzed using a Mouse IAA (Insulin Auto-Antibodies) ELISA Kit (Fine Test). This analysis revealed that most mice displayed high concentrations of anti-insulin antibodies compared to pre-diabetic animals (FIG. 18). While no statistically significant difference was observed among the different populations, mice of the COMBO arm displayed a trend towards lower levels. Mice of the 71D6 arm could be clearly divided into 2 subpopulations with low and high auto-antibodies levels, respectively. While these results warrant further investigation, they overall strengthen the hypothesis that neither anti-CD3 antibodies nor 71D6 treatment affect the production of auto-antibodies in this system, but rather act downstream to prevent or delay the onset of diabetes.

Altogether, the data obtained in this set of experiments suggest that 71D6 treatment is very effective in maintaining pancreatic beta cell integrity in the context of type 1 diabetes. Not only systemic 71D6 treatment was significantly more effective than an established immune-suppressing therapy, but it also increased the efficacy of the latter when administered in combination. The mechanism of action underlying the therapeutic activity of 71D6 seems to be related to its ability to promote beta cell survival and/or proliferation rather than interfering with the production of auto-antibodies or the infiltration of immune cells into the pancreatic islets. These data provide experimental evidence that MET-agonistic antibodies can be used in the treatment of type 1 diabetes, alone or in combination with immune therapy.

### Example 21: In Vivo Activity: Promotion of Glucose Uptake and Cooperation with Insulin in a Mouse Model of Type I Diabetes

HGF has been reported to promote insulin-dependent glucose uptake in cultured mouse skeletal muscle cells (Perdomo et al., J Biol Chem. 283, 13700-13706, 2008). We therefore tested whether our agonistic anti-MET antibodies could reduce high blood glucose levels in a mouse model of type I diabetes. To this end, we induced pancreatic β-cell degeneration in 7 week-old female BALB/c mice (Charles River) by i.p. injection of streptozotocin (STZ; Sigma Aldrich). STZ was injected at a dose of 40 mg/kg every day for 5 consecutive days. One week after the last injection, blood glucose levels under fasting conditions were determined using standard glucose strips (GIMA). At this time, STZ-treated mice displayed a mean basal glycemy two times higher compared to untreated mice (240 mg/dL vs. 120 mg/dL). Mice were randomized into 4 arms of 7 mice each based on basal glycemy, which received treatment with purified 71G3, 71D6, 71G2 or vehicle only (PBS), respectively. Antibodies were administered two times a week by i.p. injection at a dose of 1 mg/kg. An additional, fifth control arm contained 7 mice that received no STZ or antibody and served as healthy control. Blood glucose concentration in fasting conditions was monitored over time for 5 weeks. At the end of week 5, we performed a glucose tolerance test (GTT) and an insulin tolerance test (ITT). A GTT consists in administering glucose to a fasting animal by oral gavage and then measuring blood glucose levels at different time points. An ITT consists in administering insulin to a partially fasting animal by i.p or i.v. injection and then measuring blood glucose levels at different time points.

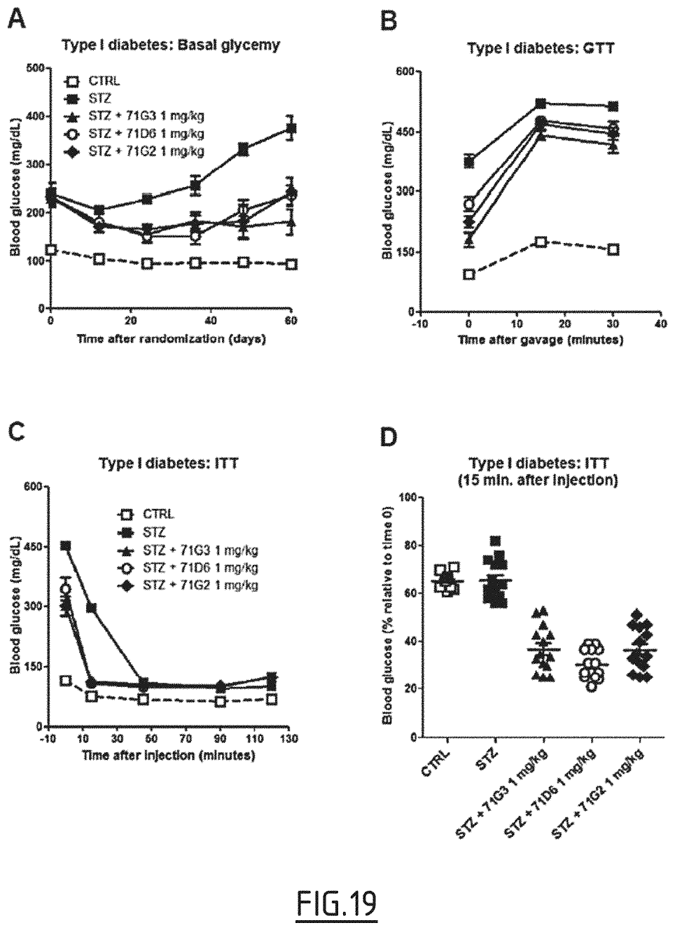

As shown in FIG. 19A, basal blood glucose levels in STZ-treated mice continued to increase for the whole duration of the experiment. This is due to chronic pancreas inflammation, which progressively aggravates organ injury. In contrast, antibody-treated animals displayed steadily decreasing glycemic levels which eventually reached a plateau after the second week of treatment. Antibody administration did not completely normalize glycemy but lowered it by up to 25%, thus bringing it about half way between the levels observed in STZ-treated mice and in control mice. Considering that in this model hyperglycemy is due to the absence of β-cell-derived insulin, we wondered whether lower glucose levels in the antibody arms was due to increased insulin levels. However, ELISA assays on blood samples revealed that this is not the case (not shown). In a GTT, mice receiving antibody treatment—while starting from lower blood glucose levels—failed to display a normal glucose uptake curve (FIG. 19B). In contrast, antibody-treated mice did display a more rapid response to insulin in an ITT (FIG. 19C). Fifteen minutes after insulin injection, glucose blood levels in mice subjected to chronic antibody treatment dropped to approximately 30-40% relative to time zero, which is significantly less than what observed in both STZ-treated mice and control animals (FIG. 190). These results suggest that agonistic anti-MET antibodies promote glucose uptake in the absence of insulin. They also suggest that agonistic anti-MET antibodies and insulin, when both are present, cooperate in mediating glucose uptake.

This hypothesis was tested in cell-based assays using mouse skeletal muscle cells. C2C12 mouse myoblast cells (obtained from American Tissue Type Collection) were induced to differentiate into myocytes as recommended by the provider and then incubated with human/mouse equivalent agonistic anti-MET antibodies (71G3, 71D6, 71G2). After 24 hours, antibody-treated cells were divided into 3 arms, which were subjected to acute stimulation with 0 nM, 100 nM or 1000 nM human recombinant insulin (Sigma Aldrich) for 1 hour in the presence of the fluorescent glucose analogue 2-(N-(7-Nitrobenz-2-oxa-1,3-diazol-4-yl)Amino)-2-Deoxyglucose (2-NBDG; Life Technologies). 2-NBDG uptake was determined by flow cytometry.

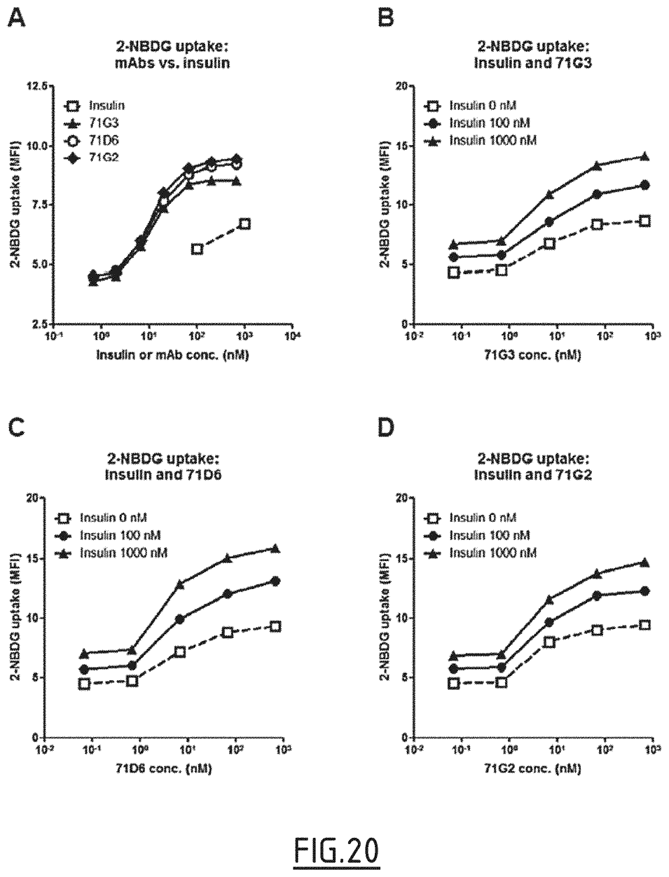

As shown in FIG. 20, 71G3, 71D6 and 71G2 promoted glucose uptake in a dose-dependent fashion. Combination of insulin and agonistic anti-MET antibodies resulted in a co-operative effect and promoted higher glucose uptake compared to both insulin alone and antibodies alone. These data are consistent with the finding that HGF and insulin cooperate in regulating glucose metabolism in cultured cells (Fafalios et al. Nat Med. 17, 1577-1584, 2011), and confirm our hypothesis that agonistic anti-MET antibodies are capable of enhancing both insulin-independent and -dependent glucose uptake.

### Example 22: In Vivo Activity: Blood Glucose Level Normalization and Insulin Resistance Overcoming in a Mouse Model of Type II Diabetes

Prompted by the observation that human/mouse equivalent agonistic anti-MET antibodies could cooperate with insulin in promoting glucose uptake, we tested their therapeutic potential in a mouse model of type II diabetes. Type II diabetes mellitus is characterized by high blood glucose levels, hyperinsulinemia, and insulin resistance. One of the most characterized mouse models of type II diabetes is represented by db/db mice, a C57BLKS/J strain bearing a point mutation in the leptin receptor gene lepr. This mutation results in loss of satiety sense and thus in unlimited feeding, leading to obesity and the above mentioned type II diabetes clinical hallmarks (reviewed by Wang et al. Curr Diabetes Rev. 10, 131-145, 2014).

Female db/db mice were obtained from Charles River (JAX™ Mice Strain BKS.Cg-Dock7^{m}+/+Lepr^{db}J) at the age of 7 weeks. One week later, animals were randomized into 4 arms of 5 mice each, which received treatment with purified 71G3, 71D6, 71G2 or vehicle only (PBS), respectively. Antibodies were administered two times a week by i.p. injection at a dose of 1 mg/kg. Blood glucose concentration in fasting conditions was monitored every 14 days for 8 weeks. After 7 weeks of treatment, i.e. when mice were 15 weeks old, a glucose tolerance test (GTT) and an insulin tolerance test (ITT) were performed.

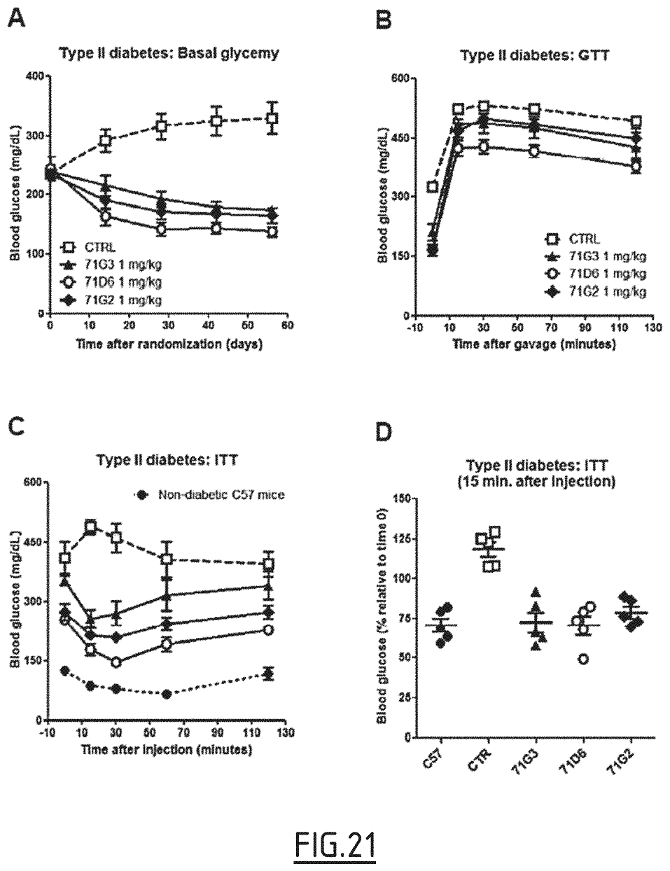

As shown in FIG. 21, the mean basal blood glucose concentration in the PBS arm at the time of randomization was approximately 230 mg/dL, which definitely corresponds to diabetic levels. These values tended to increase over time and at the end of the experiment, i.e. 8 weeks later, the mean blood glucose concentration in the PBS arm was approximately 330 mg/dL. In contrast, in the arms receiving antibody treatment, basal glycemy in fasting conditions decreased constantly over time. At the end of the experiment, the mean blood glucose concentration in the 71G3, 71D6 and 71G2 arms was 173 mg/dL, 138 mg/dL and 165 mg/dL, respectively.

After 7 weeks of treatment, i.e. when mice were 15 weeks old, we tested their acute response to glucose and insulin challenge. It should be kept in mind that these mice, in contrast to mice treated with STZ (see example 21), are hyperinsulinemic and display high blood glucose levels because they are insulin-resistant. In fact, when challenged with glucose in a GTT, mice of the PBS arm failed to display a normal glucose uptake profile. All mice showed a sharp increase in glycemy that remained elevated for the whole duration of the test. Symmetrically, when subjected to an ITT, the same mice showed a paradoxical response to insulin, displaying a slight and transient increase in glucose levels. This paradoxical response is a hallmark of insulin resistance, at least in pre-clinical models.

Mice of the antibody arms, while starting from lower basal levels, also did not display a normal glucose uptake profile in a GTT, thus suggesting that agonistic anti-MET antibodies are unable to neutralize an acute burst in blood glucose levels. However, remarkably, antibody treatment did dramatically improve response to insulin in an ITT, reversing the paradoxical effect observed in the PBS arm and making the ITT profile look more similar to that displayed by non-diabetic mice (C57BLKS/J; Charles River). We conclude that long-term treatment with agonistic anti-MET antibodies ameliorates type II diabetes in db/db mice and partially overcomes insulin resistance.

Based on these results and those presented in the previous example, we suggest that human/mouse equivalent agonistic anti-MET antibodies may be used in the clinic to treat pathological conditions associated with high blood glucose levels. These may include type I diabetes mellitus, type II diabetes mellitus, or other diabetes-like pathologies that are characterized by high glucose and/or insulin resistance (e.g. metabolic syndrome).

### Example 23: In Vivo Activity: Wound Healing in Diabetic Mice

A clinically relevant complication of diabetes is represented by increased ulceration and impaired healing of wounds. Since HGF has been implicated in wound healing (Nakamura et al., J Gastroenterol Hepatol. 1, 188-202, 2011), we sought to determine whether human/mouse equivalent anti-MET antibodies could promote the healing of wounds in a diabetic background. To this end, we obtained db/db diabetic mice as described above. At the age of 8 weeks, we subjected animals to anaesthesia and then cut a 0.8 cm-wide circular wound in the right posterior flank using a circular punch blade for skin biopsies (GIMA). The entire epidermal layer was removed. The day after surgery, mice were randomized into 4 arms that received treatment with purified 71G3, 71D6 and 71G2 or vehicle only (PBS). Antibodies were delivered every second day by i.p injection at a dose of 5 mg/kg. Wound diameter was measured every day using a calliper.

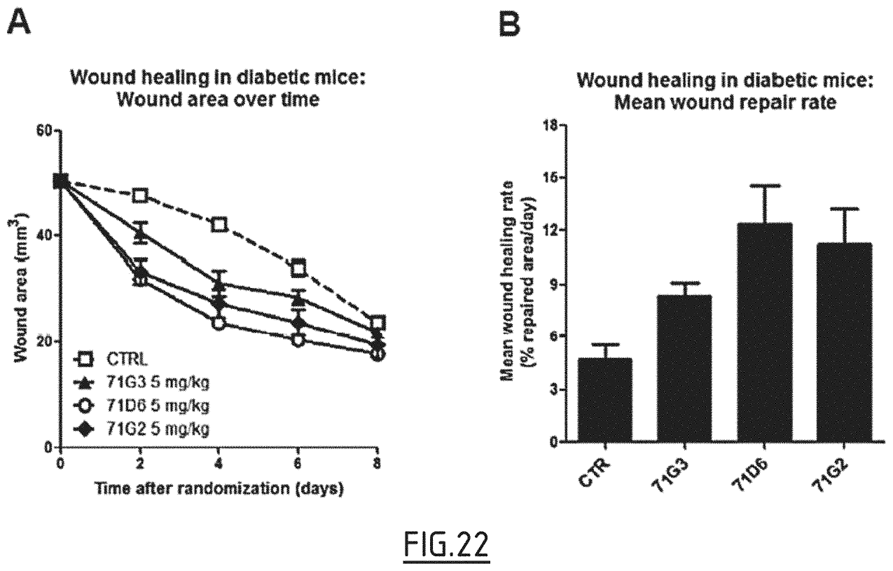

As shown in FIG. 22, antibody treatment significantly accelerated wound closure and re-epithelization. While the control arm repaired the experimental wound at an average rate of 5% per day, this value increased to 8% in the 71G3 arm, to 12% in the 71D6 arm, and to 11% in the 71G2 arm.

We suggest that human/mouse equivalent agonistic anti-MET antibodies could be used in the clinic to treat diabetes-associated ulcers and wounds that typically display impaired healing. Diabetes-associated sores represent an unmet medical need. In the United States, diabetes is the leading cause of non-traumatic lower extremity amputations. Agonistic anti-MET antibodies may be used to accelerate healing, improve re-epithelization and promote vascularization of high blood glucose-induced sores.

What is claimed is:

1. A method of increasing pancreatic islet cell growth comprising administering to a subject an HGF-MET agonist.

2. The method according to claim 1 wherein the method is used to promote insulin production or treat diabetes in a subject in need thereof.

3. The method according to claim 1 wherein the method is used to treat diabetes-associated ulcers and wounds.

4. The method according to claim 1, wherein the subject exhibits a fasting glucose level of greater than 5.6 mmol/L.

5. The method according to claim 1, wherein the subject has a population of pancreatic islet cells ranging at least about 50% smaller than the population in a healthy individual to about 80% smaller.

6. The method according to claim 1, wherein the subject has type 1 diabetes, type 2 diabetes, or has previously received a pancreatic tissue transplant.

7. The method according to claim 1, further comprising administering a pancreatic tissue transplant to the subject, or administering one or more immunosuppressive agents to the subject.

8. The method according to claim 6, wherein the one or more immunosuppressive agents are selected from the group consisting of: an anti-CD3 antibody, an anti-IL-21 antibody, a CTLA4 molecule, a PD-L1 molecule, IL-10, and Glutamic Acid Decarboxylase (GAD)-65.

9. The method according to claim 1, wherein the subject is a healthy donor of pancreatic islet cells.

10. The method according to claim 1, wherein administration of the HGF-MET agonist promotes growth of pancreatic islet beta cells.

11. The method of claim 1, wherein the HGF-MET agonist is administered at a dose in the range from 0.1-40 mg/kg per dose.

12. The method of claim 1 wherein the HGF-MET agonist is administered at a dose of 1 mg/kg, 3 mg/kg, 10 mg/kg, or 30 mg/kg.

13. The method according to claim 1, wherein the HGF-MET agonist is administered 1-3 times per week.

14. The method according to claim 1 wherein the method further comprises administering insulin or other anti-diabetes medication to the subject.

15. The method according to claim 1 wherein the HGF-MET agonist is an anti-MET agonist antibody or antigen-binding fragment thereof.

16. The method according to claim 15 wherein the anti-MET antibody or antigen-binding fragment thereof binds to a material selected from the group consisting of a SEMA domain of MET, blades 4-5 of the SEMA β-propeller, and an epitope comprising a residue selected from the group consisting of Ile367 and Asp372 of MET.

17. The method according to claim 15 wherein the anti-MET antibody or antigen-binding fragment thereof binds to the PSI domain of MET and/or binds an epitope between residues 546 and 562 of MET.

18. The method according to claim 15 wherein the anti-MET antibody or antigen-binding fragment thereof binds to an epitope comprising residue Thr555 of MET.

19. The method according to claim 15 wherein the anti-MET agonist antibody or antigen-binding fragment comprises the combination of HCDR1 consisting of SEQ ID NO: 30, HCDR2 consisting of SEQ ID NO: 32, HCDR3 consisting of SEQ ID NO: 34, LCDR1 consisting of SEQ ID NO: 107, LCDR2 consisting of SEQ ID NO: 109, and LCDR3 consisting of SEQ ID NO: 111.

20. The method according to claim 15 wherein the anti-MET agonist antibody or antigen-binding fragment comprises a VH domain at least 90% identical to SEQ ID NO: 163 and/or comprises a VL domain at least 90% identical to SEQ ID NO: 164.

21. The method according to claim 15, wherein the anti-MET agonist antibody is selected from the group consisting of an agonist antibody or antigen-binding fragment comprises a VH domain consisting of SEQ ID NO: 163 and comprises a VL domain consisting of SEQ ID NO: 164 and an IgG4 antibody.

22. The method according to claim 15 further comprising administering insulin to the subject at least daily.

23. A pharmaceutical composition capable of being used in the method according to claim 1, wherein the pharmaceutical composition comprises an HGF-MET agonist and a pharmaceutically acceptable excipient or carrier.

24. An in vitro method of promoting growth of a cell population or tissue comprising pancreatic islet cells, the method comprising contacting the cell population or tissue with an HGF-MET agonist.

25. An ex vivo method of preserving an islet cell or pancreas transplant which comprises contacting the islet cell or pancreas transplant with an HGF-MET agonist.
